(19) United States
(12) Patent Application Publication
(10) Pub. No.: US 2025/0129177 A1
(43) Pub. Date: Apr. 24, 2025

(54) Combination Therapy Involving Anti-CD39 Antibodies and Anti-PD-1 or Anti-PD-L1 Antibodies

(71) Applicant: Trishula Therapeutics, Inc., South San Francisco, CA (US)

(72) Inventors: Courtney BEERS, South San Francisco, CA (US); Achim MOESTA, South San Francisco, CA (US)

(21) Appl. No.: 18/945,361

(22) Filed: Jan. 10, 2025

Related U.S. Application Data
(63) Continuation of application No. 17/432,412, filed on Aug. 19, 2021.
(60) Provisional application No. 62/808,714, filed on Feb. 21, 2019.

(51) Int. Cl. C07K 16/28; A61K 39/00; A61P 35/00

(52) U.S. Cl. CPC C07K 16/2896; A61P 35/00; C07K 16/2818; C07K 16/2827; A61K 2039/505

(57) ABSTRACT

Provided herein are combination therapies involving antibodies with binding specificity for CD39 and antibodies with binding specificity for PD-1 and/or PD-L1.

### RELATED APPLICATION

This application is a continuation of U.S. patent application Ser. No. 17/432,412, filed Aug. 19, 2021, which claims benefit of and priority to International Application No. PCT/US2020/019320, filed Feb. 21, 2020, which claims benefit of and priority to U.S. provisional application No. 62/808,714, filed Feb. 21, 2019, the disclosure of each of which is incorporated by reference herein in its entirety.

### SEQUENCE LISTING

The instant application contains a Sequence Listing which has been submitted electronically in XML format and is hereby incorporated by reference in its entirety. Said XML copy, created on Jan. 8, 2025, is named 110738500101_SL.xml and is 486,264 bytes in size.

### FIELD

Provided herein are combination therapies involving antibodies with binding specificity for CD39 and antibodies with binding specificity for PD-1 and/or PD-L1.

### BACKGROUND

Human CD39 is a 510-amino acid protein with seven potential N-linked glycosylation sites, 11 cysteine residues, and two transmembrane regions. CD39 is an integral membrane protein that phosphohydrolyzes ATP to yield ADP and AMP. Structurally, it is characterized by two transmembrane domains, small cytoplasmic domains, and a large extracellular hydrophobic domain. CD39 becomes catalytically active upon localization to the cell surface.

CD39 is constitutively expressed in spleen, thymus, lung, and placenta and in these tissues it is associated primarily with endothelial cells and immune cell populations, such as B cells, natural killer (NK) cells, dendritic cells (DC), Langerhans cells, monocytes, macrophages, mesangial cells, neutrophils, and regulatory T cells (Tregs). Expression of CD39 on CD8⁺ and CD4⁺ T cells can also be induced upon activation and within the tumor microenvironment. Given that CD39, along with other enzymes, degrades ATP, ADP, and AMP to adenosine, CD39 can be viewed as an immunological switch that shifts ATP-driven pro-inflammatory immune cell activity toward an anti-inflammatory state mediated by adenosine.

The expression of CD39 is increased in many solid tumors. For example CD39 expression is increased in colorectal cancer, head and neck cancer, pancreatic cancer, bladder cancer, brain cancer, breast cancer, gastric cancer, hepatocellular carcinoma, lung cancer, non-small cell lung cancer, chronic lymphocytic leukemia, lymphoma, melanoma, ovarian cancer, and prostate cancer. Increased CD39 expression suggests that the enzyme is involved in the development and progression of malignancies. Expression of CD39 in solid tumors may be found on the tumor epithelium, on infiltrating leukocyte populations or on the vascular endothelium.

PD-1 is a membrane protein of 268 amino acids. PD-1 includes an extracellular IgV domain, a transmembrane region, and an intracellular tail. The tail contains two phosphorylation sites located in an immunoreceptor tyrosine-based inhibitory motif and an immunoreceptor tyrosine-based switch motif. It has been suggested that PD-1 negatively regulates T-cell receptor signals.

PD-1 is moderately expressed on naive T cells, B cells, and NK cells and up-regulated by T/B cell receptor signaling on lymphocytes, monocytes, and myeloid cells. PD-1 has a role in regulating the immune system's response by down-regulating the immune system and promoting self-tolerance by suppressing T cell inflammatory activity. This prevents autoimmune diseases, but it can also prevent the immune system from killing cancer cells.

PD-1 is recognized as an important player in immune regulation and the maintenance of peripheral tolerance. PD-1 can be viewed as an immune checkpoint and operates through multiple different mechanisms. For example, PD-1 promotes apoptosis of antigen-specific T-cells in lymph nodes. Further, PD-1 reduces apoptosis in regulatory T cells (anti-inflammatory, suppressive T cells).

PD-1 binds two ligands, PD-L1 and PD-L2. Both PD-1 ligands are members of the CD28-B7 family of co-signaling molecules that play important roles throughout all stages of T-cell function and other cell functions. The interaction of PD-1 and its ligands sends a signal into the T cell and essentially switches it off or inhibits it.

Cancer cells take advantage of this system by driving high levels of expression of PD-L1. This allows cancer cells to gain control of the PD-1 pathway and switch off T cells expressing PD-1 thus suppressing the anticancer immune response. PD-L1 is correlated with poor prognosis in ovarian, renal, colorectal, pancreatic, liver cancers, and melanoma. Similarly, PD-1 expression on tumor infiltrating lymphocytes shows dysfunctional T cells in breast cancer and melanoma and correlates with poor prognosis in renal cancer.

PD-1 therapies which ‘unblock’ an existing immune response or which unblock the initiation of an immune response are effective but sometimes only a subgroup of subjects responds. In addition, even in the responding population the response is not always complete or optimal. Modulators of CD39 may provide additional potential therapies for these types of cancers.

The efficacy of an immunological switch and a checkpoint inhibitor can be enhanced if used in combination with one another. In particular, efficacy of CD39 and PD-1 or PD-L1 antibodies may be enhanced if administered in combination with each other.

### SUMMARY

Provided herein are methods and pharmaceuticals compositions for treatment of a subject suffering from cancer comprising a therapeutically effective amount of an antibody which binds to CD39 and a therapeutically effective amount of an antibody which binds to PD-1 or PD-L1.

In some embodiments, the antibody which binds to CD39 comprises or consists of a heavy chain variable region (VH) and a light chain variable region (VL), with VH and/or VL comprising 1, 2, 3, 4, 5, or 6 of:
a) a VHCDR1 having the sequence set forth in any one of SEQ ID NOs: 1-21 or SEQ ID NOs: 315-319,
b) a VHCDR2 having the sequence set forth in any one of SEQ ID NOs: 32-50 or SEQ ID NOs: 321-325,
c) a VHCDR3 having the sequence set forth in any one of SEQ ID NOs: 58-85 or SEQ ID NOs: 327-331,
d) a VLCDR1 having the sequence set forth in any one of SEQ ID NOs: 93-107 or SEQ ID NOs: 333-337,
c) a VLCDR2 having the sequence set forth in any one of SEQ ID NOs: 115-130 or SEQ ID NOs: 339-343, and
f) a VLCDR3 having the sequence set forth in any one of SEQ ID NOs: 138-163 or SEQ ID NOs: 345-349.
   In some embodiments, the antibody that binds to CD39 comprises or consists of a heavy chain variable region (VH) and a light chain variable region (VL), with VH comprising, consisting of, or consisting essentially of a VH having any one of the sequences set forth in SEQ ID NOs: 171-210, 351, 355, 359, 363, or 367 and a VL comprising, consisting of, or consisting essentially of a VL having any one of the sequences set forth in SEQ ID NOs: 218-247, 352, 356, 360, 364, or 368. In some embodiments, the antibody that binds to CD39 comprises or consists of a heavy chain variable region and a light chain variable region, with VH comprising, consisting of, or consisting essentially of a VH having the sequence set forth in SEQ ID NO: 172 and a VL comprising, consisting of, or consisting essentially of a VL having the sequence set forth in SEQ ID NO: 219. In some embodiments, the antibody that binds to CD39 comprises or consists of a heavy chain variable region and a light chain variable region, with VH comprising, consisting of, or consisting essentially of a VH having the sequence set forth in SEQ ID NO: 351 and a VL comprising, consisting of, or consisting essentially of a VL having the sequence set forth in SEQ ID NO: 352.

In some embodiments, the antibody that binds to PD-1 or PD-L1 comprises or consists of a heavy chain variable region (VH) and a light chain variable region (VL), VH and/or VL comprising 1, 2, 3, 4, 5, or 6 of:
a) a VHCDR1 having the sequence set forth in SEQ ID NO: 25-31,
b) a VHCDR2 having the sequence set forth in SEQ ID NO: 51-57,
c) a VHCDR3 having the sequence set forth in SEQ ID NO: 86-92,
d) a VLCDR1 having the sequence set forth in SEQ ID NO: 108-114,
c) a VLCDR2 having the sequence set forth in SEQ ID NO: 131-137, and
f) a VLCDR3 having the sequence set forth in SEQ ID NO: 164-170.
   In some embodiments, the antibody that binds to PD-1 or PD-L1 comprises or consists of a heavy chain variable region (VH) and a light chain variable region (VL), with VH comprising, consisting of, or consisting essentially of a VH having the one of the sequences set forth in SEQ ID NOS: 211-217 and with VL a comprising, consisting of, or consisting essentially of a VL having one of the sequences set forth in SEQ ID NO: 248-254. In some embodiments, the antibody that binds to PD-1 comprises or consists of a heavy chain variable region (VH) and a light chain variable region (VL), with VH comprising, consisting of, or consisting essentially of a VH having the sequence set forth in SEQ ID NO: 211 and a VL comprising, consisting of, or consisting essentially of a VL having the sequence set forth in SEQ ID NO: 248.

In some embodiments, the antibody that binds to CD39 comprises or consists of a heavy chain variable region (VH) and a light chain variable region (VL), with VH comprising, consisting of, or consisting essentially of a VH having the sequence set forth in SEQ ID NO: 172 and with VL comprising, consisting of, or consisting essentially of a VL having the sequence set forth in SEQ ID NO: 219 and the antibody that binds to PD-1 comprises or consists of a heavy chain variable region and a light chain variable region, with VH comprising, consisting of, or consisting essentially of a VH having the sequence set forth in SEQ ID NO: 211 and with VL comprising, consisting of, or consisting essentially of a VL having the sequence set forth in SEQ ID NO: 248.

The combination of antibodies shows combinatorial effects in a tumor model. For example, the combination of an anti-CD39 antibody and an anti-PD-1 antibody increased the number of complete responses compared to a monotherapy with only one of the antibodies. Further, subjects, such as animals, with the combination therapy of an anti-CD-39 antibody and an anti-PD-1 antibody were resistant to tumor challenge.

In some embodiments, the cancer is a solid cancer. In some embodiments, the cancer is a hematological cancer. In some embodiments, the cancer is selected from the group consisting of metastatic non-small cell lung cancer (NSCLC), metastatic head and neck squamous cell carcinoma (HNSCC), melanoma, renal cell carcinoma, metastatic cutaneous squamous cell carcinoma, Hodgkin's lymphoma, and unresectable or metastatic solid tumor with DNA mismatch repair deficiencies or a microsatellite instability-high state. In some embodiments, the subject is recurrent or progressive after platinum therapy. In some embodiments, the subject is a human subject.

In some embodiments, the method enhances pro-inflammatory cytokine secretion in an assay. In some embodiments, the cytokines are one or more of the cytokines selected from IL-2, IFN-γ, or TNF-α. In some embodiments, the method enhances T-cell proliferation and/or cytotoxicity in an assay. In some embodiments, the T cells comprise or consist of CD4⁺ cells and/or CD8⁺ cells. In some embodiments, the assay comprises a one-way MLR or a two-way MLR. In some embodiments, the assay comprises a stimulated T cell assay.

### BRIEF DESCRIPTION OF THE DRAWINGS

FIG. 1 shows that combined treatment of an anti-CD39 antibody and an anti-PD-1 antibody enhances (A) CD4⁺ T-cell proliferation and (B) CD8⁺ T-cell proliferation in a one-way MLR. The x-axis depicts the type of antibody used and the y-axis shows % CD4⁺ proliferation or % CD8⁺ proliferation, respectively. The dotted lines indicate the percent proliferation of cells treated with only anti-CD39. Error bars depict Standard Deviation.

FIG. 2 shows that (A) combined treatment of an anti-CD39 antibody and an anti-PD-1 antibody enhances pro-inflammatory cytokine secretion in a one-way MLR. Cytokine type is depicted above each drawing. The x-axis depicts the type of antibody used and the y-axis depicts the amount of cytokine. The dotted lines indicate the percent proliferation of cells treated with only anti-CD39. Error bars depict Standard Deviation. (B). In some instances, the anti-PD-1 antibody is pembrolizumab and the assay may be carried in the presence of 100 μM ATP.

FIG. 3 shows that combination treatment with an anti-CD39 antibody and an anti-PD-L1 antibody enhances (A) CD8⁺ T-cell proliferation and (B) pro-inflammatory cytokine production. The dotted lines indicate the percent proliferation (A) or cytokine concentration (B) of cells treated with only anti-CD39.

FIG. 4 shows that combined treatment of an (A) anti-CD39 antibody and an anti-PD-1 antibody enhances pro-inflammatory cytokine secretion in a two-way MLR. Each panel depicts a unique alloreactive donor pair. The x-axis depicts the type of antibody used and the y-axis depicts the amount of IL-2. Error bars depict Standard Deviation. The top dotted line depicts IL-2 secretion from an anti-CD39 treated sample and the bottom dotted line depicts IL-2 secretion with an isotype-matched control antibody. (B) shows that combined treatment of an anti-CD39 antibody and an anti-PD-1 antibody enhances pro-inflammatory cytokine secretion in a two-way MLR when the anti-CD39 antibody is SRF360 and the anti-PD-1 antibody is pembrolizumab.

FIG. 5 shows that combined treatment of an anti-CD39 antibody and an anti-PD-1 antibody enhances T-cell proliferation and pro-inflammatory cytokine secretion from a stimulated PBMC in the presence of ATP. The x-axis depicts the type of antibody used and the absence of ATP. The y-axis on the left panel depicts % CD8⁺ proliferation and the y-axis on the right panel depicts amount of TNF-α. Error bars depict Standard Deviation. The top dotted line designates a no ATP control and the bottom dotted line designates isotype control.

FIG. 6 shows that anti-CD39 in combination with an anti-PD-L1 therapy enhances anti-tumor responses in an MC38 syngenic model. Curves depict mean tumor volume, with error bars indicating the standard error of the mean (SEM). The vertical dashed lines indicate dosing days after the second randomization. The vertical dashed lines indicate drug combination dosing on days 8, 12, 15, and 19. The x-axis shows days after implantation and the y-axis show tumor volume.

FIG. 7 shows an anti-CD39 antibody demonstrates single agent activity and combinatorial effects with an anti-PD-1 antibody in the CT26 syngeneic tumor model. Curves depict mean tumor volume with error bars indicating the SEM. The vertical dashed lines indicate dosing days after the second randomization. The vertical dashed lines indicate drug combination dosing on days 8, 12, 15, and 19. The x-axis shows days after implantation and the y-axis show tumor volume. CR=complete response.

FIG. 8 shows animals with complete responses following monotherapy with an anti-CD39 antibody and combination therapy with an anti-CD39 antibody and an anti-PD-1 antibody were resistant to tumor challenge. Curves depict mean tumor volume for animals that were rechallenged only: 5 tumor naive animals (starting at day 49), n=3 animals with prior anti-CD39 antibody treatment and n=8 animals with prior anti-CD39 and anti-PD-1 combination treatment. Error bars indicated the SEM. The x-axis shows days after implantation and the y-axis show tumor volume.

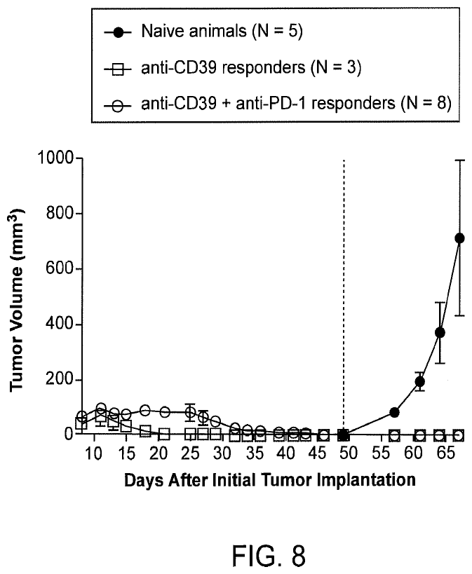

### DETAILED DESCRIPTION

Provided herein are combination therapies involving antibodies with binding specificity for CD39 and antibodies with binding specificity for PD-1 and/or PD-L1.

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

The term “combinations thereof” includes every possible combination of elements to which the term refers.

The terms “CD39,” “CD39 antigen,” and “Cluster of Differentiation 39” are used interchangeably herein. CD39 is also known as also known as ectonucleoside triphosphate diphosphohydrolase-1 (gene: ENTPD1; protein: NTPDase1, see www.ncbi.nlm.nih.gov/gene/953). CD39 has also been referred to as ATPDase and SPG64. Each of the terms set forth above may be used interchangeably. Unless specified otherwise, the terms include any variants, isoforms, and species homologs of human CD39 that are naturally expressed by cells, or that are expressed by cells transfected with a CD39 gene. In some embodiments, CD39 proteins include murine CD39. In some embodiments, CD39 proteins include cynomolgus CD39.

The terms “PD-1,” “programmed cell death protein 1,” and “Cluster of Differentiation 279” are used interchangeably herein. Unless specified otherwise, the terms include any variants, isoforms, and species homologs of human PD-1 that are naturally expressed by cells, or that are expressed by cells transfected with a PD-1 gene. In some embodiments, PD-1 proteins include murine PD-1.

The terms “PD-L1,” “programmed death-ligand 1,” “Cluster of Differentiation 274,” “B7 homolog 1,” and “B7-H1” are used interchangeably herein. Unless specified otherwise, the terms include any variants, isoforms, and species homologs of human PD-L1 that are naturally expressed by cells, or that are expressed by cells transfected with a PD-L1 gene. In some embodiments, PD-L1 proteins include murine PD-L1.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, CH1, CH2, and CH3. Each light chain typically comprises a light chain variable region (VL) and a light chain constant region. The light chain constant region typically comprises one domain, abbreviated CL.

The term “antibody” describes a type of immunoglobulin molecule and is used herein in its broadest sense. An antibody specifically includes intact antibodies (e.g., intact immunoglobulins) and antibody fragments. Antibodies comprise at least one antigen-binding domain. One example of an antigen-binding domain is an antigen binding domain formed by a VH-VL dimer.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ¿, γ, and u, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Unless otherwise specified, the numbering scheme used for identification of a particular CDR herein is the Kabat numbering scheme. Variant and equivalent antibodies with a Chothia numbering scheme are intended to be within the scope of the invention.

TABLE 1
Residues in CDRs according to Kabat and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments. In some embodiments, an antibody that binds CD39, an antibody that binds PD-1, and/or an antibody that binds PD-L1 includes antibody fragments of each of an antibody that binds CD39, an antibody that binds PD-1, and/or an antibody that binds PD-L1.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by papain digestion of a full-length antibody.

“F(ab′)₂” fragments contain two Fab′ fragments joined, near the hinge region, by disulfide bonds. F(ab′)₂ fragments may be generated, for example, by pepsin digestion of an intact antibody. The F(ab′) fragments can be dissociated, for example, by treatment with B-mercaptoethanol.

“Single-chain Fv” or “sFv” or “scFv” antibody fragments comprise a VH domain and a VL domain in a single polypeptide chain. The VH and VL are generally linked by a peptide linker. See Pluckthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety. “scFv-Fc” fragments comprise an scFv attached to an Fc domain. For example, an Fc domain may be attached to the C-terminal of the scFv. The Fc domain may follow the V_{H} or V_{L} depending on the orientation of the variable domains in the scFv (i.e., V_{H}-V_{L} or V_{L}-V_{H}). Any suitable Fc domain known in the art or described herein may be used.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaccous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some embodiments, an isolated antibody is prepared by at least one purification step.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Affinity can be determined, for example, using surface plasmon resonance (SPR) technology, such as a Biacore® instrument.

With regard to the binding of an antibody to a target molecule, the terms “binding” or “binds to” a particular antigen (e.g., a polypeptide target) or an epitope on a particular antigen mean binding that is measurably different from a non-selective interaction. Binding can be measured, for example, by determining binding of a molecule compared to binding of a control molecule. Binding can also be determined by competition with a control molecule that is similar to the target, such as an excess of non-labeled target. In that case, binding is indicated if the binding of the labeled target to a probe is competitively inhibited by the excess non-labeled target.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M⁻¹), as used herein, refers to the association equilibrium constant of a particular antibody-antigen interaction. K_{A}=kₐ/k_{d}.

Percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of one or more amino acids with one or more chemically or functionally similar amino acids. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the following groups of amino acids are considered conservative substitutions for one another.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, |
| | T, V, W, and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in Turn | A, C, D, E, G, H, K, N, Q, |
| Formation | R, S, P, and T |
| Flexible Residues | Q, T, K, S, G, P, D, E, and R |
| Group 1 | A, S, and T |
| Group 2 | D and E |
| Group 3 | N and Q |
| Group 4 | R and K |
| Group 5 | I, L, and M |
| Group 6 | F, Y, and W |
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any cancer refers, in certain embodiments, to ameliorating a cancer that exists in a subject. In another embodiment, “treating” or “treatment” includes ameliorating at least one physical parameter, which may be indiscernible by the subject. In yet another embodiment, “treating” or “treatment” includes modulating the cancer, either physically (e.g., stabilization of a discernible symptom) or physiologically (e.g., stabilization of a physical parameter) or both.

As used herein, the term “therapeutically effective amount” or “effective amount” refers to an amount of an antibody or composition that when administered to a subject is effective to treat a cancer. In some embodiments, a therapeutically effective comprises or consists of exemplary doses of each antibody. In some embodiments, a therapeutically effective amount comprises or consists of determining an amount used to achieve a response according to a clinical endpoint. In some embodiments, the clinical endpoint comprises Objective Response Rate (ORR), Progression Free Survival (PFS), and/or Response Evaluation Criteria in Solid Tumors (“RECIST”).

As used herein, the term “subject” means a mammal or a human. In some embodiments subjects include, but are not limited to, monkeys, dogs, cats, mice, rats, cows, horses, camels, avians, goats, and sheep.

### 2. Antibody Combinations

Provided herein are methods and antibody combinations for the treatment of cancer. The antibody combinations combine an antibody that binds CD39 and an antibody that binds PD-1 and/or PD-L1.

A first aspect provides a method for treatment of a subject suffering from cancer, comprising administering to the subject a therapeutically effective amount of an antibody which binds to CD39 and a therapeutically effective amount of an antibody which binds to PD-1 and/or PD-L1.

In some embodiments, the antibody that binds to CD39 comprises or consists of a heavy chain variable region (VH) and a light chain variable region (VL), with VH and/or VL comprising:
a) a VHCDR1 having the sequence set forth in any one of SEQ ID NOs: 1-21 or SEQ ID NOs: 315-319,
b) a VHCDR2 having the sequence set forth in any one of SEQ ID NOs: 32-50 or SEQ ID NOs: 321-325,
c) a VHCDR3 having the sequence set forth in any one of SEQ ID NOs: 58-85 or SEQ ID NOs: 327-331,
d) a VLCDR1 having the sequence set forth in any one of SEQ ID NOs: 93-107 or SEQ ID NOs: 333-337,
e) a VLCDR2 having the sequence set forth in any one of SEQ ID NOs: 115-130 or or SEQ ID NOs: 339-343, and
f) a VLCDR3 having the sequence set forth in any one of SEQ ID NOs: 138-163 or SEQ ID NOs: 345-349.
   In some embodiments, the antibody that binds to CD39 comprises or consists of a heavy chain variable region (VH) and a light chain variable region (VL), with VH comprising, consisting of, or consisting essentially of a VH having any one of the sequence set forth in SEQ ID NOs: 171-210, 351, 355, 359, 363, or 367 and a with VL comprising, consisting of, or consisting essentially of a VL having any one of the sequences set forth in SEQ ID NOs: 218-247, 352, 356, 360, 364, or 368. In some embodiments, the antibody that binds to CD39 comprises or consists of a heavy chain variable region (VH) and a light chain variable region (VL), with VH comprising, consisting of, or consisting essentially of a VH having the sequence set forth in SEQ ID NO: 172 and with VL comprising, consisting of, or consisting essentially of a VL having the sequence set forth in SEQ ID NO: 219. In some embodiments, the antibody that binds to CD39 comprises or consists of a heavy chain variable region and a light chain variable region, with VH comprising, consisting of, or consisting essentially of a VH having the sequence set forth in SEQ ID NO: 351 and a VL comprising, consisting of, or consisting essentially of a VL having the sequence set forth in SEQ ID NO: 352.

In some embodiments, the antibody that binds to PD-1 comprises or consists of a heavy chain variable region (VH) and a light chain variable region (VL), VH and/or VL comprising:
a) a VHCDR1 having the sequence set forth in SEQ ID NO: 25,
b) a VHCDR2 having the sequence set forth in SEQ ID NO: 51,
c) a VHCDR3 having the sequence set forth in SEQ ID NO: 86,
d) a VLCDR1 having the sequence set forth in SEQ ID NO: 108,
e) a VLCDR2 having the sequence set forth in SEQ ID NO: 131, and
f) a VLCDR3 having the sequence set forth in SEQ ID NO: 164.
   In some embodiments, the antibody that binds to PD-1 comprises or consists of a heavy chain variable region (VH) and a light chain variable region (VL), with VH comprising, consisting of, or consisting essentially of a VH having the sequence set forth in SEQ ID NO: 211 and a with VL comprising, consisting of, or consisting essentially of a VL having the sequence set forth in SEQ ID NO: 248.

In some embodiments, the antibody that binds to PD-1 comprises or consists of a heavy chain variable region (VH) and a light chain variable region (VL), VH and/or VL comprising:
a) a VHCDR1 having the sequence set forth in SEQ ID NO: 26,
b) a VHCDR2 having the sequence set forth in SEQ ID NO: 52,
c) a VHCDR3 having the sequence set forth in SEQ ID NO: 87,
d) a VLCDR1 having the sequence set forth in SEQ ID NO: 109,
e) a VLCDR2 having the sequence set forth in SEQ ID NO: 132, and
f) a VLCDR3 having the sequence set forth in SEQ ID NO: 165.

In some embodiments, the antibody that binds to PD-1 comprises or consists of a heavy chain variable region (VH) and a light chain variable region (VL), with VH comprising, consisting of, or consisting essentially of a VH having the sequence set forth in SEQ ID NO: 212 and a with VL comprising, consisting of, or consisting essentially of a VL having the sequence set forth in SEQ ID NO: 249.

In some embodiments, the antibody that binds to PD-1 comprises or consists of a heavy chain variable region (VH) and a light chain variable region (VL), VH and/or VL comprising:
a) a VHCDR1 having the sequence set forth in SEQ ID NO: 27,
b) a VHCDR2 having the sequence set forth in SEQ ID NO: 53,
c) a VHCDR3 having the sequence set forth in SEQ ID NO: 88,
d) a VLCDR1 having the sequence set forth in SEQ ID NO: 110,
e) a VLCDR2 having the sequence set forth in SEQ ID NO: 133, and
f) a VLCDR3 having the sequence set forth in SEQ ID NO: 166.
   In some embodiments, the antibody that binds to PD-1 comprises or consists of a heavy chain variable region (VH) and a light chain variable region (VL), with VH comprising, consisting of, or consisting essentially of a VH having the sequence set forth in SEQ ID NO: 213 and a with VL comprising, consisting of, or consisting essentially of a VL having the sequence set forth in SEQ ID NO: 250.

In some embodiments, the antibody that binds to PD-1 comprises or consists of a heavy chain variable region (VH) and a light chain variable region (VL), VH and/or VL comprising:
a) a VHCDR1 having the sequence set forth in SEQ ID NO: 28,
b) a VHCDR2 having the sequence set forth in SEQ ID NO: 54,
c) a VHCDR3 having the sequence set forth in SEQ ID NO: 89,
d) a VLCDR1 having the sequence set forth in SEQ ID NO: 111,
e) a VLCDR2 having the sequence set forth in SEQ ID NO: 134, and
f) a VLCDR3 having the sequence set forth in SEQ ID NO: 167.
   In some embodiments, the antibody that binds to PD-1 comprises or consists of a heavy chain variable region (VH) and a light chain variable region (VL), with VH comprising, consisting of, or consisting essentially of a VH having the sequence set forth in SEQ ID NO: 214 and a with VL comprising, consisting of, or consisting essentially of a VL having the sequence set forth in SEQ ID NO: 251.

In some embodiments, the antibody that binds to PD-L1 comprises or consists of a heavy chain variable region (VH) and a light chain variable region (VL), VH and/or VL comprising:
a) a VHCDR1 having the sequence set forth in SEQ ID NO: 29,
b) a VHCDR2 having the sequence set forth in SEQ ID NO: 55,
c) a VHCDR3 having the sequence set forth in SEQ ID NO: 90,
d) a VLCDR1 having the sequence set forth in SEQ ID NO: 112,
e) a VLCDR2 having the sequence set forth in SEQ ID NO: 135, and
f) a VLCDR3 having the sequence set forth in SEQ ID NO: 168.
   In some embodiments, the antibody that binds to PD-L1 comprises or consists of a heavy chain variable region (VH) and a light chain variable region (VL), with VH comprising, consisting of, or consisting essentially of a VH having the sequence set forth in SEQ ID NO: 215 and a with VL comprising, consisting of, or consisting essentially of a VL having the sequence set forth in SEQ ID NO: 252.

In some embodiments, the antibody that binds to PD-L1 comprises or consists of a heavy chain variable region (VH) and a light chain variable region (VL), VH and/or VL comprising:
a) a VHCDR1 having the sequence set forth in SEQ ID NO: 30,
b) a VHCDR2 having the sequence set forth in SEQ ID NO: 56,
c) a VHCDR3 having the sequence set forth in SEQ ID NO: 91,
d) a VLCDR1 having the sequence set forth in SEQ ID NO: 113,
e) a VLCDR2 having the sequence set forth in SEQ ID NO: 136, and
f) a VLCDR3 having the sequence set forth in SEQ ID NO: 169.
   In some embodiments, the antibody that binds to PD-L1 comprises or consists of a heavy chain variable region (VH) and a light chain variable region (VL), with VH comprising, consisting of, or consisting essentially of a VH having the sequence set forth in SEQ ID NO: 216 and a with VL comprising, consisting of, or consisting essentially of a VL having the sequence set forth in SEQ ID NO: 253.

In some embodiments, the antibody that binds to PD-L1 comprises or consists of a heavy chain variable region (VH) and a light chain variable region (VL), VH and/or VL comprising:
a) a VHCDR1 having the sequence set forth in SEQ ID NO: 31,
b) a VHCDR2 having the sequence set forth in SEQ ID NO: 57,
c) a VHCDR3 having the sequence set forth in SEQ ID NO: 92,
d) a VLCDR1 having the sequence set forth in SEQ ID NO: 114,
e) a VLCDR2 having the sequence set forth in SEQ ID NO: 137, and
f) a VLCDR3 having the sequence set forth in SEQ ID NO: 170.
   In some embodiments, the antibody that binds to PD-L1 comprises or consists of a heavy chain variable region (VH) and a light chain variable region (VL), with VH comprising, consisting of, or consisting essentially of a VH having the sequence set forth in SEQ ID NO: 217 and a with VL comprising, consisting of, or consisting essentially of a VL having the sequence set forth in SEQ ID NO: 254.

In some embodiments, the antibody that binds to CD39 comprises or consists of a heavy chain variable region (VH) and a light chain variable region (VL), with VH comprising, consisting of, or consisting essentially of a VH having the sequence set forth in SEQ ID NO: 172 and VL comprising, consisting of, or consisting essentially of a VL having the sequence set forth in SEQ ID NO: 219 and the antibody that binds to PD-1 comprises or consists of a heavy chain variable region and a light chain variable region, with VH comprising, consisting of, or consisting essentially of a VH having the sequence set forth in SEQ ID NO: 211 and VL comprising, consisting of, or consisting essentially of a VL having the sequence set forth in SEQ ID NO: 248.

In some embodiments, the cancer is a solid cancer. In some embodiments, the cancer is a hematological cancer. In some embodiments, the cancer is selected from the group consisting of metastatic non-small cell lung cancer (NSCLC), metastatic head and neck squamous cell carcinoma (HNSCC), melanoma, renal cell carcinoma, metastatic cutaneous squamous cell carcinoma, Hodgkin's lymphoma, and unresectable or metastatic solid tumor with DNA mismatch repair deficiencies or a microsatellite instability-high state. In some embodiments, the subject is recurrent or progressive after platinum therapy. In some embodiments, the subject is a human subject.

In some embodiments, the method enhances pro-inflammatory cytokine secretion in an assay. In some embodiments, the cytokines are one or more of the cytokines selected from IL-2, IFN-γ, or TNF-α. In some embodiments, the method enhances T-cell proliferation and/or cytotoxicity in an assay. In some embodiments, the T cells comprise or consist of CD4⁺ cells and/or CD8⁺ cells. In some embodiments, the assay comprises a one-way MLR or a two-way MLR. In some embodiments, the assay comprises a stimulated T cell assay.

A second aspect provides a pharmaceutical composition comprising a therapeutically effective amount of an antibody which binds to CD39 and a therapeutically effective amount of at least one the antibody that binds PD-1 or PD-L1.

In some embodiments, the antibody that binds to CD39 comprises or consists of a heavy chain variable region (VH) and a light chain variable region (VL), with VH and/or VL comprising:
a) a VHCDR1 having the sequence set forth in any one of SEQ ID NOs: 1-21 or SEQ ID NOs: 315-319,
b) a VHCDR2 having the sequence set forth in any one of SEQ ID NOs: 32-50 or SEQ ID NOs: 321-325,
c) a VHCDR3 having the sequence set forth in any one of SEQ ID NOs: 58-85 or SEQ ID NOs: 327-331,
d) a VLCDR1 having the sequence set forth in any one of SEQ ID NOs: 93-107 or SEQ ID NOs: 333-337,
e) a VLCDR2 having the sequence set forth in any one of SEQ ID NOs: 115-130 or SEQ ID NOs: 339-343, and
f) a VLCDR3 having the sequence set forth in any one of SEQ ID NOs: 138-163 or SEQ ID NOs: 345-349.
   In some embodiments, the antibody that binds to CD39 comprises or consists of a heavy chain variable region (VH) and a light chain variable region (VL), with VH comprising, consisting of, or consisting essentially of a VH having the sequence set forth in any one of SEQ ID NOs: 171-210, 351, 355, 359, 363, or 367 and with VL comprising, consisting of, or consisting essentially of a VL having the sequence set forth in any one of SEQ ID NOs: 218-247, 352, 356, 360, 364, or 368. In some embodiments, the antibody that binds to CD39 comprises or consists of a heavy chain variable region (VH) and a light chain variable region (VL), with VH comprising, consisting of, or consisting essentially of a VH having the sequence set forth in SEQ ID NO: 172 and with VL comprising, consisting of, or consisting essentially of a VL having the sequence set forth in SEQ ID NO: 219. In some embodiments, the antibody that binds to CD39 comprises or consists of a heavy chain variable region and a light chain variable region, with VH comprising, consisting of, or consisting essentially of a VH having the sequence set forth in SEQ ID NO: 351 and a VL comprising, consisting of, or consisting essentially of a VL having the sequence set forth in SEQ ID NO: 352.

In some embodiments, the antibody that binds to PD-1 comprises or consists of a heavy chain variable region (VH) and a light chain variable region (VL), VH and/or VL comprising:
a) a VHCDR1 having the sequence set forth in SEQ ID NO: 25,
b) a VHCDR2 having the sequence set forth in SEQ ID NO: 51,
c) a VHCDR3 having the sequence set forth in SEQ ID NO: 86,
d) a VLCDR1 having the sequence set forth in SEQ ID NO: 108,
e) a VLCDR2 having the sequence set forth in SEQ ID NO: 131, and
f) a VLCDR3 having the sequence set forth in SEQ ID NO: 164.
   In some embodiments, the antibody that binds to PD-1 comprises or consists of a heavy chain variable region (VH) and a light chain variable region (VL), with VH comprising, consisting of, or consisting essentially of a VH having the sequence set forth in SEQ ID NO: 211 and a with VL comprising, consisting of, or consisting essentially of a VL having the sequence set forth in SEQ ID NO: 248.

In some embodiments, the antibody that binds to PD-1 comprises or consists of a heavy chain variable region (VH) and a light chain variable region (VL), VH and/or VL comprising:
a) a VHCDR1 having the sequence set forth in SEQ ID NO: 26,
b) a VHCDR2 having the sequence set forth in SEQ ID NO: 52,
c) a VHCDR3 having the sequence set forth in SEQ ID NO: 87,
d) a VLCDR1 having the sequence set forth in SEQ ID NO: 109,
e) a VLCDR2 having the sequence set forth in SEQ ID NO: 132, and
f) a VLCDR3 having the sequence set forth in SEQ ID NO: 165.

In some embodiments, the antibody that binds to PD-1 comprises or consists of a heavy chain variable region (VH) and a light chain variable region (VL), with VH comprising, consisting of, or consisting essentially of a VH having the sequence set forth in SEQ ID NO: 212 and a with VL comprising, consisting of, or consisting essentially of a VL having the sequence set forth in SEQ ID NO: 249.

In some embodiments, the antibody that binds to PD-1 comprises or consists of a heavy chain variable region (VH) and a light chain variable region (VL), VH and/or VL comprising:
a) a VHCDR1 having the sequence set forth in SEQ ID NO: 27,
b) a VHCDR2 having the sequence set forth in SEQ ID NO: 53,
c) a VHCDR3 having the sequence set forth in SEQ ID NO: 88,
d) a VLCDR1 having the sequence set forth in SEQ ID NO: 110,
e) a VLCDR2 having the sequence set forth in SEQ ID NO: 133, and
f) a VLCDR3 having the sequence set forth in SEQ ID NO: 166.

In some embodiments, the antibody that binds to PD-1 comprises or consists of a heavy chain variable region (VH) and a light chain variable region (VL), with VH comprising, consisting of, or consisting essentially of a VH having the sequence set forth in SEQ ID NO: 213 and a with VL comprising, consisting of, or consisting essentially of a VL having the sequence set forth in SEQ ID NO: 250.

In some embodiments, the antibody that binds to PD-1 comprises or consists of a heavy chain variable region (VH) and a light chain variable region (VL), VH and/or VL comprising:
a) a VHCDR1 having the sequence set forth in SEQ ID NO: 28,
b) a VHCDR2 having the sequence set forth in SEQ ID NO: 54,
c) a VHCDR3 having the sequence set forth in SEQ ID NO: 89,
d) a VLCDR1 having the sequence set forth in SEQ ID NO: 111,
e) a VLCDR2 having the sequence set forth in SEQ ID NO: 134, and a VLCDR3 having the sequence set forth in SEQ ID NO: 167.

In some embodiments, the antibody that binds to PD-1 comprises or consists of a heavy chain variable region (VH) and a light chain variable region (VL), with VH comprising, consisting of, or consisting essentially of a VH having the sequence set forth in SEQ ID NO: 214 and a with VL comprising, consisting of, or consisting essentially of a VL having the sequence set forth in SEQ ID NO: 251.

In some embodiments, the antibody that binds to PD-L1 comprises or consists of a heavy chain variable region (VH) and a light chain variable region (VL), VH and/or VL comprising:
a) a VHCDR1 having the sequence set forth in SEQ ID NO: 29,
b) a VHCDR2 having the sequence set forth in SEQ ID NO: 55,
c) a VHCDR3 having the sequence set forth in SEQ ID NO: 90,
d) a VLCDR1 having the sequence set forth in SEQ ID NO: 112,
e) a VLCDR2 having the sequence set forth in SEQ ID NO: 135, and
f) a VLCDR3 having the sequence set forth in SEQ ID NO: 168.

In some embodiments, the antibody that binds to PD-L1 comprises or consists of a heavy chain variable region (VH) and a light chain variable region (VL), with VH comprising, consisting of, or consisting essentially of a VH having the sequence set forth in SEQ ID NO: 215 and a with VL comprising, consisting of, or consisting essentially of a VL having the sequence set forth in SEQ ID NO: 252.

In some embodiments, the antibody that binds to PD-L1 comprises or consists of a heavy chain variable region (VH) and a light chain variable region (VL), VH and/or VL comprising:
a) a VHCDR1 having the sequence set forth in SEQ ID NO: 30,
b) a VHCDR2 having the sequence set forth in SEQ ID NO: 56,
c) a VHCDR3 having the sequence set forth in SEQ ID NO: 91,
d) a VLCDR1 having the sequence set forth in SEQ ID NO: 113,
e) a VLCDR2 having the sequence set forth in SEQ ID NO: 136, and
f) a VLCDR3 having the sequence set forth in SEQ ID NO: 169.

In some embodiments, the antibody that binds to PD-L1 comprises or consists of a heavy chain variable region (VH) and a light chain variable region (VL), with VH comprising, consisting of, or consisting essentially of a VH having the sequence set forth in SEQ ID NO: 216 and a with VL comprising, consisting of, or consisting essentially of a VL having the sequence set forth in SEQ ID NO: 253.

In some embodiments, the antibody that binds to PD-L1 comprises or consists of a heavy chain variable region (VH) and a light chain variable region (VL), VH and/or VL comprising:
a) a VHCDR1 having the sequence set forth in SEQ ID NO: 31,
b) a VHCDR2 having the sequence set forth in SEQ ID NO: 57,
c) a VHCDR3 having the sequence set forth in SEQ ID NO: 92,
d) a VLCDR1 having the sequence set forth in SEQ ID NO: 114,
e) a VLCDR2 having the sequence set forth in SEQ ID NO: 137, and
f) a VLCDR3 having the sequence set forth in SEQ ID NO: 170.

In some embodiments, the antibody that binds to PD-L1 comprises or consists of a heavy chain variable region (VH) and a light chain variable region (VL), with VH comprising, consisting of, or consisting essentially of a VH having the sequence set forth in SEQ ID NO: 217 and a with VL comprising, consisting of, or consisting essentially of a VL having the sequence set forth in SEQ ID NO: 254.

In some embodiments, the antibody that binds to CD39 comprises or consists of a heavy chain variable region (VH) and a light chain variable region (VL), with VH comprising, consisting of, or consisting essentially of a VH having the sequence set forth in SEQ ID NO: 172 and VL comprising, consisting of, or consisting essentially of a VL having the sequence set forth in SEQ ID NO: 219 and the antibody that binds to PD-1 comprises or consists of a heavy chain variable region and a light chain variable region, with VH comprising, consisting of, or consisting essentially of a VH having the sequence set forth in SEQ ID NO: 211 and VL comprising, consisting of, or consisting essentially of a VL having the sequence set forth in SEQ ID NO: 248.

In some embodiments, the method enhances pro-inflammatory cytokine secretion in an assay. In some embodiments, the cytokines are one or more of the cytokines selected from IL-2, IFN-γ, or TNF-α. In some embodiments, the method enhances T-cell proliferation and/or cytotoxicity in an assay. In some embodiments, the T cells comprise or consist of CD4⁺ cells and/or CD8⁺ cells. In some embodiments, the assay comprises a one-way MLR or a two-way MLR. In some embodiments, the assay comprises a stimulated T cell assay.

### CDR-H1+CDR-H2+CDR-H3 Regions of the Antibodies

In some embodiments, the antibody that binds to CD39 comprises a VH sequence comprising a CDR-H1 sequence comprising, consisting of, or consisting essentially of a sequence selected from SEQ ID NOs: 1-21 or SEQ ID NOs: 315-319, a CDR-H2 sequence comprising, consisting of, or consisting essentially of a sequence selected from SEQ ID NOs: 32-50 or SEQ ID NOs: 321-325, and a CDR-H3 sequence comprising, consisting of, or consisting essentially of a sequence selected from SEQ ID NOs: 58-85 or SEQ ID NOs: 327-331. In some embodiments, the CDR-H1 sequence, CDR-H2 sequence, and the CDR-H3 sequence are all from a single illustrative VH sequence provided in this disclosure. For example, in some embodiments, the CDR-H1, CDR-H2, and CDR-H3 are all from a single illustrative VH sequence selected from SEQ ID NOs: 171-210, SEQ ID NO: 351, SEQ ID NO: 355, SEQ ID NO: 359, SEQ ID NO: 363, or SEQ ID NO: 367.

In some embodiments, the antibody that binds to CD39 comprises a VH sequence comprising a CDR-H1 sequence comprising SEQ ID NO: 2, a CDR-H2 sequence comprising SEQ ID NO: 33, and a CDR-H3 sequence comprising SEQ ID NO: 59.

In some embodiments, the antibody that binds to PD-1 comprises a VH sequence comprising a CDR-H1 sequence comprising, consisting of, or consisting essentially of a sequence selected from SEQ ID NOs: 25-28, a CDR-H2 sequence comprising, consisting of, or consisting essentially of a sequence selected from SEQ ID NOs: 51-54, and a CDR-H3 sequence comprising, consisting of, or consisting essentially of a sequence selected from SEQ ID NOs: 86-89. In some embodiments, the CDR-H1 sequence, CDR-H2 sequence, and the CDR-H3 sequence are all from a single illustrative VH sequence provided in this disclosure. For example, in some embodiments, the CDR-H1, CDR-H2, and CDR-H3 are all from a single illustrative VH sequence selected from SEQ ID NOs: 211-214.

In some embodiments, the antibody that binds to PD-1 comprises a VH sequence comprising a CDR-H1 sequence comprising, consisting of, or consisting essentially of a sequence selected from SEQ ID NO: 25, a CDR-H2 sequence comprising, consisting of, or consisting essentially of a sequence selected from SEQ ID NO: 51, and a CDR-H3 sequence comprising, consisting of, or consisting essentially of a sequence selected from SEQ ID NO: 86. In some embodiments, the antibody that binds to PD-1 comprises a VH sequence comprising a CDR-H1 sequence comprising, consisting of, or consisting essentially of a sequence selected from SEQ ID NO: 26, a CDR-H2 sequence comprising, consisting of, or consisting essentially of a sequence selected from SEQ ID NO: 52, and a CDR-H3 sequence comprising, consisting of, or consisting essentially of a sequence selected from SEQ ID NO: 87. In some embodiments, the antibody that binds to PD-1 comprises a VH sequence comprising a CDR-H1 sequence comprising, consisting of, or consisting essentially of a sequence selected from SEQ ID NO: 27, a CDR-H2 sequence comprising, consisting of, or consisting essentially of a sequence selected from SEQ ID NO: 53, and a CDR-H3 sequence comprising, consisting of, or consisting essentially of a sequence selected from SEQ ID NO: 88. In some embodiments, the antibody that binds to PD-1 comprises a VH sequence comprising a CDR-H1 sequence comprising, consisting of, or consisting essentially of a sequence selected from SEQ ID NO: 28, a CDR-H2 sequence comprising, consisting of, or consisting essentially of a sequence selected from SEQ ID NO: 54, and a CDR-H3 sequence comprising, consisting of, or consisting essentially of a sequence selected from SEQ ID NO: 89.

In some embodiments, the antibody that binds to PD-L1 comprises a VH sequence comprising a CDR-H1 sequence comprising, consisting of, or consisting essentially of a sequence selected from SEQ ID NOs: 29-31, a CDR-H2 sequence comprising, consisting of, or consisting essentially of a sequence selected from SEQ ID NOs: 55-57, and a CDR-H3 sequence comprising, consisting of, or consisting essentially of a sequence selected from SEQ ID NOs: 90-92. In some embodiments, the CDR-H1 sequence, CDR-H2 sequence, and the CDR-H3 sequence are all from a single illustrative VH sequence provided in this disclosure. For example, in some embodiments, the CDR-H1, CDR-H2, and CDR-H3 are all from a single illustrative VH sequence selected from SEQ ID NOs: 215-217.

In some embodiments, the antibody that binds to PD-L1 comprises a VH sequence comprising a CDR-H1 sequence comprising, consisting of, or consisting essentially of a sequence selected from SEQ ID NO: 29, a CDR-H2 sequence comprising, consisting of, or consisting essentially of a sequence selected from SEQ ID NO: 55, and a CDR-H3 sequence comprising, consisting of, or consisting essentially of a sequence selected from SEQ ID NO: 90. In some embodiments, the antibody that binds to PD-L1 comprises a VH sequence comprising a CDR-H1 sequence comprising, consisting of, or consisting essentially of a sequence selected from SEQ ID NO: 30, a CDR-H2 sequence comprising, consisting of, or consisting essentially of a sequence selected from SEQ ID NO: 56, and a CDR-H3 sequence comprising, consisting of, or consisting essentially of a sequence selected from SEQ ID NO: 91. In some embodiments, the antibody that binds to PD-L1 comprises a VH sequence comprising a CDR-H1 sequence comprising, consisting of, or consisting essentially of a sequence selected from SEQ ID NO: 31, a CDR-H2 sequence comprising, consisting of, or consisting essentially of a sequence selected from SEQ ID NO: 57, and a CDR-H3 sequence comprising, consisting of, or consisting essentially of a sequence selected from SEQ ID NO: 92.

In some embodiments, the antibody that binds to CD39 comprises a VH sequence comprising a CDR-H1 sequence comprising SEQ ID NO: 2, a CDR-H2 sequence comprising SEQ ID NO: 33, and a CDR-H3 sequence comprising SEQ ID NO: 59 and the antibody that binds to PD-1 comprises a VH sequence comprising a CDR-H1 sequence comprising SEQ ID NO: 25, a CDR-H2 sequence comprising SEQ ID NO: 51, and a CDR-H3 sequence comprising SEQ ID NO: 86.

### V_{H} Sequences

In some embodiments, the antibody that binds CD39 comprises a VH sequence comprising, consisting of, or consisting essentially of a sequence selected from SEQ ID NOs: 171-210, SEQ ID NO: 351, SEQ ID NO: 355, SEQ ID NO: 359, SEQ ID NO: 363, or SEQ ID NO: 367. In some embodiments, the antibody that binds CD39 comprises a VH sequence comprising, consisting of, or consisting essentially of SEQ ID NO: 172.

In some embodiments, the antibody that binds PD-1 comprises a VH sequence comprising, consisting of, or consisting essentially of SEQ ID NO: 211. In some embodiments, the antibody that binds PD-1 comprises a VH sequence comprising, consisting of, or consisting essentially of SEQ ID NO: 212. In some embodiments, the antibody that binds PD-1 comprises a VH sequence comprising, consisting of, or consisting essentially of SEQ ID NO: 213. In some embodiments, the antibody that binds PD-1 comprises a VH sequence comprising, consisting of, or consisting essentially of SEQ ID NO: 214.

In some embodiments, the antibody that binds PD-L1 comprises a VH sequence comprising, consisting of, or consisting essentially of SEQ ID NO: 215. In some embodiments, the antibody that binds PD-L1 comprises a VH sequence comprising, consisting of, or consisting essentially of SEQ ID NO: 216. In some embodiments, the antibody that binds PD-L1 comprises a VH sequence comprising, consisting of, or consisting essentially of SEQ ID NO: 217.

In some embodiments, the antibody that binds CD39 comprises a VH sequence comprising, consisting of, or consisting essentially of SEQ ID NO: 172 and the antibody that binds PD-1 comprises a VH sequence comprising, consisting of, or consisting essentially of SEQ ID NO: 211.

### CDR-L1+CDR-L2+CDR-L3 Regions of the Antibody

In some embodiments, the antibody which binds to CD39 comprises a V_{L} sequence comprising a CDR-L1 sequence comprising, consisting of, or consisting essentially of a sequence selected from SEQ ID NOs: 93-107 or SEQ ID NOs 333-337, a CDR-L2 sequence comprising, consisting of, or consisting essentially of a sequence selected from SEQ ID NOs: 115-130 or SEQ ID NOs: 339-343, and a CDR-L3 sequence comprising, consisting of, or consisting essentially of a sequence selected from SEQ ID NOs: 138-163 or SEQ ID NOs: 345-349. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence selected from SEQ ID NOs: 218-247, SEQ ID NO: 352, SEQ ID NO: 356, SEQ ID NO: 360, SEQ ID NO: 364, or SEQ ID NO: 368. In some embodiments, the antibody which binds to CD39 comprises a V_{L} sequence comprising a CDR-L1 sequence comprising SEQ ID NO: 94, a CDR-L2 sequence comprising SEQ ID NO: 116, and a CDR-L3 sequence comprising SEQ ID NO: 139.

In some embodiments, the antibody which binds to PD-1 comprises a V_{L} sequence comprising a CDR-L1 sequence comprising, consisting of, or consisting essentially of a sequence selected from SEQ ID NOs: 108-111, a CDR-L2 sequence comprising, consisting of, or consisting essentially of a sequence selected from SEQ ID NOs: 131-134, and a CDR-L3 sequence comprising, consisting of, or consisting essentially of a sequence selected from SEQ ID NOs: 164-167. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence selected from SEQ ID NOs: 248-251.

In some embodiments, the antibody which binds to PD-1 comprises a V_{L} sequence comprising a CDR-L1 sequence comprising SEQ ID NO: 108, a CDR-L2 sequence comprising SEQ ID NO: 131, and a CDR-L3 sequence SEQ ID NO: 164. In some embodiments, the antibody which binds to PD-1 comprises a VL sequence comprising a CDR-L1 sequence comprising SEQ ID NO: 109, a CDR-L2 sequence comprising SEQ ID NO: 132, and a CDR-L3 sequence SEQ ID NO: 165. In some embodiments, the antibody which binds to PD-1 comprises a VL sequence comprising a CDR-L1 sequence comprising SEQ ID NO: 110, a CDR-L2 sequence comprising SEQ ID NO: 133, and a CDR-L3 sequence SEQ ID NO: 166. In some embodiments, the antibody which binds to PD-1 comprises a VL sequence comprising a CDR-L1 sequence comprising SEQ ID NO: 111, a CDR-L2 sequence comprising SEQ ID NO: 134, and a CDR-L3 sequence SEQ ID NO: 167.

In some embodiments, the antibody which binds to PD-L1 comprises a V_{L} sequence comprising a CDR-L1 sequence comprising, consisting of, or consisting essentially of a sequence selected from SEQ ID NOs: 112-114, a CDR-L2 sequence comprising, consisting of, or consisting essentially of a sequence selected from SEQ ID NOs: 135-137, and a CDR-L3 sequence comprising, consisting of, or consisting essentially of a sequence selected from SEQ ID NOs: 168-170. In some embodiments, the CDR-L1 sequence, CDR-L2 sequence, and CDR-L3 sequence are all from a single illustrative V_{L} sequence provided in this disclosure. For example, in some embodiments, the CDR-L1, CDR-L2, and CDR-L3 are all from a single illustrative V_{L} sequence selected from SEQ ID NOs: 252-254.

In some embodiments, the antibody which binds to PD-L1 comprises a VL sequence comprising a CDR-L1 sequence comprising SEQ ID NO: 112, a CDR-L2 sequence comprising SEQ ID NO: 135, and a CDR-L3 sequence SEQ ID NO: 168. In some embodiments, the antibody which binds to PD-L1 comprises a VL sequence comprising a CDR-L1 sequence comprising SEQ ID NO: 113, a CDR-L2 sequence comprising SEQ ID NO: 136, and a CDR-L3 sequence SEQ ID NO: 169. In some embodiments, the antibody which binds to PD-L1 comprises a VL sequence comprising a CDR-L1 sequence comprising SEQ ID NO: 114, a CDR-L2 sequence comprising SEQ ID NO: 137, and a CDR-L3 sequence SEQ ID NO: 170.

In some embodiments, the antibody which binds to CD39 comprises a V_{L} sequence comprising a CDR-L1 sequence comprising, consisting of, or consisting essentially of SEQ ID NO: 94, a CDR-L2 sequence comprising, consisting of, or consisting essentially of SEQ ID NO: 116, and a CDR-L3 sequence comprising, consisting of, or consisting essentially of SEQ ID NO: 139 and the antibody which binds to PD-1 comprises a V_{L} sequence comprising a CDR-L1 sequence comprising, consisting of, or consisting essentially of SEQ ID NO: 108, a CDR-L2 sequence comprising, consisting of, or consisting essentially of SEQ ID NO: 131, and a CDR-L3 sequence comprising, consisting of, or consisting essentially of SEQ ID NO: 164.

### V_{L} Sequences

In some embodiments, the antibody that binds to CD39 comprises a V_{L} sequence comprising, consisting of, or consisting essentially of a sequence selected from any one of SEQ ID NOs: 218-247, SEQ ID NO: 352, SEQ ID NO: 356, SEQ ID NO: 360, SEQ ID NO: 364, or SEQ ID NO: 368. In some embodiments, the antibody that binds to CD39 comprises a V_{L} sequence comprising, consisting of, or consisting essentially of SEQ ID NO: 219.

In some embodiments, the antibody that binds to PD-1 comprises a V_{L} sequence comprising, consisting of, or consisting essentially of a sequence selected from any one of SEQ ID NOs: 248-251. In some embodiments, the antibody that binds to PD-1 comprises a V_{L} sequence comprising, consisting of, or consisting essentially of SEQ ID NO: 248. In some embodiments, the antibody that binds to PD-1 comprises a V_{L} sequence comprising, consisting of, or consisting essentially of SEQ ID NO: 249. In some embodiments, the antibody that binds to PD-1 comprises a V_{L} sequence comprising, consisting of, or consisting essentially of SEQ ID NO: 250. In some embodiments, the antibody that binds to PD-1 comprises a V_{L} sequence comprising, consisting of, or consisting essentially of SEQ ID NO: 251.

In some embodiments, the antibody that binds to PD-L1 comprises a V_{L} sequence comprising, consisting of, or consisting essentially of a sequence selected from any one of SEQ ID NOs: 218-247. In some embodiments, the antibody that binds to PD-L1 comprises a V_{L} sequence comprising, consisting of, or consisting essentially of SEQ ID NO: 252. In some embodiments, the antibody that binds to PD-L1 comprises a V_{L} sequence comprising, consisting of, or consisting essentially of SEQ ID NO: 253. In some embodiments, the antibody that binds to PD-L1 comprises a V_{L} sequence comprising, consisting of, or consisting essentially of SEQ ID NO: 254.

In some embodiments, the antibody that binds to CD39 comprises a V_{L} sequence comprising, consisting of, or consisting essentially of SEQ ID NO: 219 and the antibody that binds to PD-1 comprises a V_{L} sequence comprising, consisting of, or consisting essentially of SEQ ID NO: 248.

V_{H}-V_{L} Pairs

In some embodiments, the antibody which binds CD39 comprises a VH sequence and a V_{L} sequence. In some embodiments, the VH sequence is a VH sequence comprising, consisting of, or consisting essentially of any one of SEQ ID NOs: 171-210, SEQ ID NO: 351, SEQ ID NO: 355, SEQ ID NO: 359, SEQ ID NO: 363, or SEQ ID NO: 367 and the V_{L} sequence is a V_{L} sequence comprising, consisting of, or consisting essentially of any one of SEQ ID NOs: 218-247, SEQ ID NO: 352, SEQ ID NO: 356, SEQ ID NO: 360, SEQ ID NO: 364, or SEQ ID NO: 368. In some embodiments, the antibody which binds to CD39 comprises a VH sequence comprising SEQ ID NO: 172 and V_{L} sequence comprising SEQ ID NO: 219.

In some embodiments, the antibody which binds PD-1 comprises a V_{H} sequence and a V_{L} sequence. In some embodiments, the V_{H} sequence is a V_{H} sequence comprising, consisting of, or consisting essentially of any one of SEQ ID NOs: 211-214 and the V_{L} sequence is a V_{L} sequence comprising, consisting of, or consisting essentially of any one of SEQ ID NOs: 248-251.

In some embodiments, the antibody which binds to PD-1 comprises a V_{H} sequence comprising SEQ ID NO: 211 and V_{L} sequence comprising SEQ ID NO: 248. In some embodiments, the antibody which binds to PD-1 comprises a V_{H} sequence comprising SEQ ID NO: 212 and V_{L} sequence comprising SEQ ID NO: 249. In some embodiments, the antibody which binds to PD-1 comprises a V_{H} sequence comprising SEQ ID NO: 213 and V_{L} sequence comprising SEQ ID NO: 250. In some embodiments, the antibody which binds to PD-1 comprises a V_{H} sequence comprising SEQ ID NO: 214 and V_{L} sequence comprising SEQ ID NO: 251.

In some embodiments, the antibody which binds PD-L1 comprises a V_{H} sequence and a V_{L} sequence. In some embodiments, the V_{H} sequence is a V_{H} sequence comprising, consisting of, or consisting essentially of any one of SEQ ID NOs: 215-217 and the V_{L} sequence is a V_{L} sequence comprising, consisting of, or consisting essentially of any one of SEQ ID NOs: 252-254.

In some embodiments, the antibody which binds to PD-L1 comprises a V_{H} sequence comprising SEQ ID NO: 215 and V_{L} sequence comprising SEQ ID NO: 252. In some embodiments, the antibody which binds to PD-L1 comprises a V_{H} sequence comprising SEQ ID NO: 216 and V_{L} sequence comprising SEQ ID NO: 253. In some embodiments, the antibody which binds to PD-L1 comprises a V_{H} sequence comprising SEQ ID NO: 217 and V_{L} sequence comprising SEQ ID NO: 254.

In some embodiments, the antibody which binds to CD39 comprises a V_{H} sequence comprising SEQ ID NO: 172 and V_{L} sequence comprising SEQ ID NO: 219 and the antibody which binds to PD-1 comprises a V_{H} sequence comprising SEQ ID NO: 211 and a V_{L} sequence comprising SEQ ID NO: 248.

### CDR-H1+CDR-H2+CDR-H3+CDR-L1+CDR-L2+CDR-L3

In some embodiments, the antibody which binds to CD39 comprises or consists of a heavy chain variable region (V_{H}) and a light chain variable region (V_{L}), with V_{H} and/or V_{L} comprising 1, 2, 3, 4, 5, or 6 of:
a) a VHCDR1 having the sequence set forth in any one of SEQ ID NOs: 1-21 or SEQ ID NOs: 315-319,
b) a VHCDR2 having the sequence set forth in any one of SEQ ID NOs: 32-50 or SEQ ID NOs: 321-325,
c) a VHCDR3 having the sequence set forth in any one of SEQ ID NOs: 58-85 or SEQ ID NOs: 327-331,
d) a VLCDR1 having the sequence set forth in any one of SEQ ID NOs: 93-107 or SEQ ID NOs: 333-337,
e) a VLCDR2 having the sequence set forth in any one of SEQ ID NOs: 115-130 or SEQ ID NOs: 339-343, and
f) a VLCDR3 having the sequence set forth in any one of SEQ ID NOs: 138-163 or SEQ ID NOs: 345-349.

In some embodiments, the antibody which binds to CD39 comprises a V_{H} sequence comprising a CDR-H1 sequence comprising SEQ ID NO: 2, a CDR-H2 sequence comprising SEQ ID NO: 33, and a CDR-H3 sequence comprising SEQ ID NO: 59 and a V_{L} sequence comprising a CDR-L1 sequence comprising SEQ ID NO: 94, a CDR-L2 sequence comprising SEQ ID NO: 116, and a CDR-L3 sequence SEQ ID NO: 139.

In some embodiments, the antibody which binds to PD-1 comprises a V_{H} sequence comprising a CDR-H1 sequence comprising SEQ ID NO: 25, a CDR-H2 sequence comprising SEQ ID NO: 51, and a CDR-H3 sequence comprising SEQ ID NO: 86 and a VL sequence comprising a CDR-L1 sequence comprising SEQ ID NO: 108, a CDR-L2 sequence comprising SEQ ID NO: 131, and a CDR-L3 sequence SEQ ID NO: 164. In some embodiments, the antibody which binds to PD-1 comprises a V_{H} sequence comprising a CDR-H1 sequence comprising SEQ ID NO: 26, a CDR-H2 sequence comprising SEQ ID NO: 52, and a CDR-H3 sequence comprising SEQ ID NO: 87 and a VL sequence comprising a CDR-L1 sequence comprising SEQ ID NO: 109, a CDR-L2 sequence comprising SEQ ID NO: 132, and a CDR-L3 sequence SEQ ID NO: 165. In some embodiments, the antibody which binds to PD-1 comprises a V_{H} sequence comprising a CDR-H1 sequence comprising SEQ ID NO: 27, a CDR-H2 sequence comprising SEQ ID NO: 53, and a CDR-H3 sequence comprising SEQ ID NO: 88 and a VL sequence comprising a CDR-L1 sequence comprising SEQ ID NO: 110, a CDR-L2 sequence comprising SEQ ID NO: 133, and a CDR-L3 sequence SEQ ID NO: 166. In some embodiments, the antibody which binds to PD-1 comprises a V_{H} sequence comprising a CDR-H1 sequence comprising SEQ ID NO: 28, a CDR-H2 sequence comprising SEQ ID NO: 54, and a CDR-H3 sequence comprising SEQ ID NO: 89 and a VL sequence comprising a CDR-L1 sequence comprising SEQ ID NO: 111, a CDR-L2 sequence comprising SEQ ID NO: 134, and a CDR-L3 sequence SEQ ID NO: 167.

In some embodiments, the antibody which binds to PD-L1 comprises a V_{H} sequence comprising a CDR-H1 sequence comprising SEQ ID NO: 29, a CDR-H2 sequence comprising SEQ ID NO: 55, and a CDR-H3 sequence comprising SEQ ID NO: 90 and a VL sequence comprising a CDR-L1 sequence comprising SEQ ID NO: 112, a CDR-L2 sequence comprising SEQ ID NO: 135, and a CDR-L3 sequence SEQ ID NO: 168. In some embodiments, the antibody which binds to PD-L1 comprises a V_{H} sequence comprising a CDR-H1 sequence comprising SEQ ID NO: 30, a CDR-H2 sequence comprising SEQ ID NO: 56, and a CDR-H3 sequence comprising SEQ ID NO: 91 and a VL sequence comprising a CDR-L1 sequence comprising SEQ ID NO: 113, a CDR-L2 sequence comprising SEQ ID NO: 136, and a CDR-L3 sequence SEQ ID NO: 169. In some embodiments, the antibody which binds to PD-L1 comprises a V_{H} sequence comprising a CDR-H1 sequence comprising SEQ ID NO: 31, a CDR-H2 sequence comprising SEQ ID NO: 57, and a CDR-H3 sequence comprising SEQ ID NO: 92 and a VL sequence comprising a CDR-L1 sequence comprising SEQ ID NO: 114, a CDR-L2 sequence comprising SEQ ID NO: 137, and a CDR-L3 sequence SEQ ID NO: 170.

In some embodiments, the antibody which binds to CD39 comprises a V_{H} sequence comprising a CDR-H1 sequence comprising SEQ ID NO: 2, a CDR-H2 sequence comprising SEQ ID NO: 33, and a CDR-H3 sequence comprising SEQ ID NO: 59 and a VL sequence comprising a CDR-L1 sequence comprising SEQ ID NO: 94, a CDR-L2 sequence comprising SEQ ID NO: 116, and a CDR-L3 sequence SEQ ID NO: 139 and the antibody which binds to PD-1 comprises a V_{H} sequence comprising a CDR-H1 sequence comprising SEQ ID NO: 25, a CDR-H2 sequence comprising SEQ ID NO: 51, and a CDR-H3 sequence comprising SEQ ID NO: 86 and a VL sequence comprising a CDR-L1 sequence comprising SEQ ID NO: 108, a CDR-L2 sequence comprising SEQ ID NO: 131, and a CDR-L3 sequence SEQ ID NO: 164.

### HC+LC

In some embodiments, the antibody that binds CD39, PD-1, or PD-L1 comprises or consists of one or more heavy chains consisting of an HC sequence and one or more light chains consisting of an LC sequence. In some embodiments, the antibody that binds CD39, PD-1, or PD-L1 comprises or consists of two identical heavy chains consisting of an HC sequence and two identical light chains consisting of an LC sequence.

In some embodiments, the HC sequence of the antibody that binds CD39 is an HC sequence comprising, consisting of, or consisting essentially of any one of SEQ ID NOs: 255, 257, 259, 261, 263, 265, 267, 269, 271, 273, 275, 277, 279, 281, 283, 285, 287, 289, 291, 293, 295, 297, 353, 357, 361, 365, or 369 and the LC sequence of the antibody that binds CD39 is an LC sequence comprising, consisting of, or consisting essentially of any one of SEQ ID NOs: 256, 258, 260, 262, 264, 266, 268, 270, 272, 274, 276, 278, 280, 282, 284, 286, 288, 290, 292, 294, 296, 298, 354, 358, 362, 366, or 370. In some embodiments, the HC sequence of the antibody that binds CD39 is an HC sequence consisting of SEQ ID NO: 255 and the LC sequence is an LC sequence consisting of SEQ ID NO: 256. In some embodiments, the HC sequence of the antibody that binds CD39 is an HC sequence consisting of SEQ ID NO: 353 and the LC sequence is an LC sequence consisting of SEQ ID NO: 354.

In some embodiments, the HC sequence of the antibody that binds PD-1 is an HC sequence comprising, consisting of, or consisting essentially of any one of SEQ ID NO: 299, 300, 302, 304, or 306, and the LC sequence of the antibody that binds PD-1 is an LC sequence comprising, consisting of, or consisting essentially any one of SEQ ID NOs: 301, 303, 305, or 307.

In some embodiments, the HC sequence of the antibody that binds PD-1 is an HC sequence consisting of SEQ ID NO: 299 and the LC sequence is an LC sequence consisting of SEQ ID NO: 301. In some embodiments, the HC sequence of the antibody that binds PD-1 is an HC sequence consisting of SEQ ID NO: 300 and the LC sequence is an LC sequence consisting of SEQ ID NO: 301. In some embodiments, the HC sequence of the antibody that binds PD-1 is an HC sequence consisting of SEQ ID NO: 302 and the LC sequence is an LC sequence consisting of SEQ ID NO: 303. In some embodiments, the HC sequence of the antibody that binds PD-1 is an HC sequence consisting of SEQ ID NO: 304 and the LC sequence is an LC sequence consisting of SEQ ID NO: 305. In some embodiments, the HC sequence of the antibody that binds PD-1 is an HC sequence consisting of SEQ ID NO: 306 and the LC sequence is an LC sequence consisting of SEQ ID NO: 307.

In some embodiments, the HC sequence of the antibody that binds PD-L1 is an HC sequence comprising, consisting of, or consisting essentially of any one of SEQ ID NOs: 308, 310, or 312, and the LC sequence of the antibody that binds PD-L1 is an LC sequence comprising, consisting of, or consisting essentially of any one of SEQ ID NOs: 309, 311, or 313.

In some embodiments, the HC sequence of the antibody that binds PD-L1 is an HC sequence consisting of SEQ ID NO: 308 and the LC sequence is an LC sequence consisting of SEQ ID NO: 309. In some embodiments, the HC sequence of the antibody that binds PD-L1 is an HC sequence consisting of SEQ ID NO: 310 and the LC sequence is an LC sequence consisting of SEQ ID NO: 311. In some embodiments, the HC sequence of the antibody that binds PD-L1 is an HC sequence consisting of SEQ ID NO: 312 and the LC sequence is an LC sequence consisting of SEQ ID NO: 313.

In some embodiments, the HC sequence of the antibody that binds CD39 is an HC sequence comprising or consisting of SEQ ID NO: 255 and the LC sequence is an LC sequence comprising or consisting of SEQ ID NO: 256 and the HC sequence of the antibody that binds PD-1 is an HC sequence comprising or consisting of SEQ ID NO: 299 and the LC sequence is an LC sequence comprising or consisting of SEQ ID NO: 301. In some embodiments, the HC sequence of the antibody that binds CD39 is an HC sequence comprising or consisting of SEQ ID NO: 255 and the LC sequence is an LC sequence comprising or consisting of SEQ ID NO: 256 and the HC sequence of the antibody that binds PD-1 is an HC sequence comprising or consisting of SEQ ID NO: 300 and the LC sequence is an LC sequence comprising or consisting of SEQ ID NO: 301.

In some embodiments, the HC sequence of the antibody that binds CD39 is an HC sequence comprising or consisting of SEQ ID NO: 353 and the LC sequence is an LC sequence comprising or consisting of SEQ ID NO: 354 and the HC sequence of the antibody that binds PD-1 is an HC sequence comprising or consisting of SEQ ID NO: 299 and the LC sequence is an LC sequence comprising or consisting of SEQ ID NO: 301. In some embodiments, the HC sequence of the antibody that binds CD39 is an HC sequence comprising or consisting of SEQ ID NO: 353 and the LC sequence is an LC sequence comprising or consisting of SEQ ID NO: 354 and the HC sequence of the antibody that binds PD-1 is an HC sequence comprising or consisting of SEQ ID NO: 300 and the LC sequence is an LC sequence comprising or consisting of SEQ ID NO: 301.

### Glycosylation Variants

In certain embodiments, an antibody of the invention may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

In certain embodiments, the antibody is glycosylated. In certain embodiments, the antibody is deglycosylated. Carbohydrates may be removed by standard techniques. In certain embodiments, the antibody is aglycosylated, for instance by expression in a system that does not glycosylate.

### Preparation of Antibodies

### Antigen Preparation

CD39, PD-1, or PD-L1 antigens may used for production of antibodies may be intact CD39 or a fragment of CD39. The intact CD39, or fragment of CD39, may be in the form of an isolated protein or expressed by a cell. Other forms of CD39 useful for generating antibodies will be apparent to those skilled in the art.

### Monoclonal Antibodies

In some embodiments, the antibodies that bind CD39, PD-1, and/or PD-L1 are monoclonal antibodies. Monoclonal antibodies may be obtained, for example, using the hybridoma method first described by Kohler et al., Nature, 1975, 256:495-497, and/or by recombinant DNA methods (see e.g., U.S. Pat. No. 4,816,567). Monoclonal antibodies may also be obtained, for example, using phage or yeast-based libraries. See e.g., U.S. Pat. Nos. 8,258,082 and 8,691,730.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. Sec Goding J. W., Monoclonal Antibodies: Principles and Practice 3^{rd} ed. (1986) Academic Press, San Diego, CA.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### Humanized Antibodies

Humanized antibodies may be generated by replacing most, or all, of the structural portions of a monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370.

In some embodiments, the antibody that binds CD39 is a humanized anti-CD39 comprising Clone B66. In some embodiments, the antibody that binds PD-1 is a humanized anti-PD-1 comprising Clone RMP1-14. In some embodiments, the antibody that binds PD-L1 is a humanized anti-PD-L1 comprising Clone 10F.9G2.

### Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905). Human antibodies may also be generated by in vitro activated B cells (see e.g., U.S. Pat. Nos. 5,567,610 and 5,229,275). Human antibodies may also be derived from yeast-based libraries (see e.g., U.S. Pat. No. 8,691,730).

### Cancers

For cancers, the antibodies of the invention are generally administered to a human or a mammal human in a pharmaceutically acceptable dosage form. In some embodiments, the cancer is a hematological cancer. Any suitable cancer may be treated with the antibodies provided herein. In some embodiments, the cancer is a solid cancer. In some embodiments, the cancer is selected from the group consisting of metastatic non-small cell lung cancer (NSCLC), metastatic head and neck squamous cell carcinoma (HNSCC), melanoma, renal cell carcinoma, metastatic cutaneous squamous cell carcinoma, Hodgkin's lymphoma, and unresectable or metastatic solid tumor with DNA mismatch repair deficiencies or a microsatellite instability-high state. In some embodiments, the subject is recurrent or progressive after platinum therapy.

In some embodiments, the method enhances pro-inflammatory cytokine secretion in an assay. In some embodiments, the cytokines are one or more of the cytokines selected from IL-2, IFN-γ, or TNF-α. In some embodiments, the method enhances T-cell proliferation and/or cytotoxicity in an assay. In some embodiments, the T cells comprise or consist of CD4⁺ cells and/or CD8⁺ cells. In some embodiments, the assay comprises a one-way MLR or a two-way MLR. In some embodiments, the assay comprises a stimulated T cell assay.

In some embodiments, the method enhances anti-tumor responses. In some embodiments, the method enhances anti-tumor responses in a MC38 syngenic tumor model or a CT26 syngenic tumor model.

### EXAMPLES

### Example 1: Isolation and Purification of Human Immune Cells

Primary cells were directly isolated from a Leuko pak (StemCell Technologies, Vancouver, Canada) using Lymphoprep™ (Stemcell Technologies), a density gradient medium. Following isolation, the cells were cryopreserved and stored in liquid nitrogen.

### Example 2: One-Way Mixed Lymphocyte Reaction

For one-way mixed lymphocyte reactions, frozen PBMCs were thawed and monocytes were isolated using the EasySep™ Human Monocyte Isolation Kit (Stemcell Technologies) in accordance with the manufacturer's protocol. Monocytes were differentiated into Dendritic Cells (DC) with IL-4 and GM-CSF (R&D Systems, Minneapolis, MN) for 7 days.

Separately, pan T cells were isolated from an alloreactive donor using EasySep™ Human T cell Isolation Kit (Stemcell Technologies) and stained with Cell Trace Violet (CTV) (ThermoFisher, Waltham, MA). DCs were mixed with T cells and treated with 50 μg/ml of an anti-CD39 antibody (HC SEQ ID NO: 255 and LC SEQ ID NO: 256) and/or an anti-PD-1 antibody (Pembrolizumab, Merck, Kenilworth, NJ, HC SEQ ID NO: 304 and LC SEQ ID NO: 305). Alternatively the treatment was comprised of an anti-CD39 antibody and/or an anti-PD-L1 antibody (atezolizumab, Genentech, South San Francisco, CA, HC SEQ ID NO: 308 and LC SEQ ID NO: 309). ATP (Acros Organics, Geel, Belgium) was added at a final concentration of 100 μM to some reactions Supernatants were harvested after 6 days and cytokines were measured using a Meso Scale Discovery human cytokine kit (MSD, Rockville, MD). Cells were stained using fluorescently labeled anti-CD3, anti-CD8 and anti-CD4 antibodies (Biolegend, San Diego, CA). Proliferation of CD4⁺ T cells and CD8⁺ T cells was measured as the percentage of T cells undergoing division by tracing generational doubling using the CTV dye dilution method by flow cytometry.

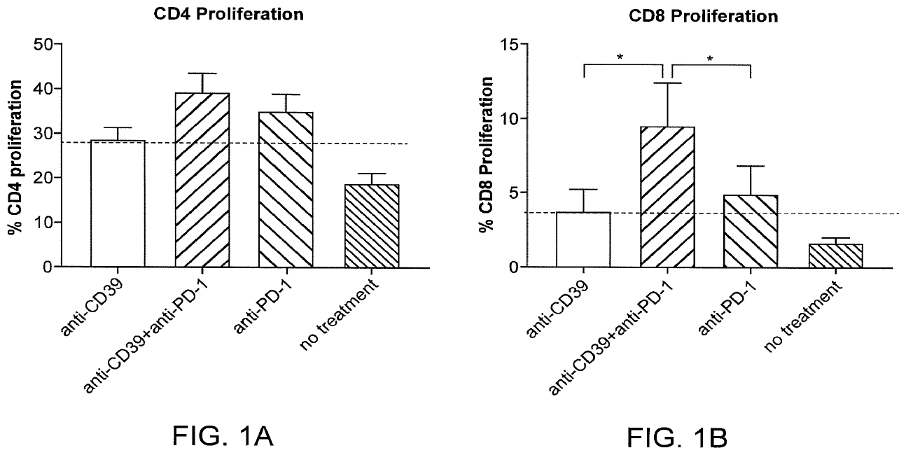

FIG. 1 shows that combined treatment of anti-CD39 and anti-PD-1 enhances (A) CD4⁺ T-cell proliferation or (B) CD8⁺ T-cell proliferation in a one-way MLR. Proliferation is depicted as the percentage of total CD4⁺ T cells or CD8⁺ T cells, respectively, having undergone division. (A) The x-axis depicts the type of antibody used and the y-axis shows % CD4⁺ T-cell proliferation or CD8⁺ T-cell proliferation, respectively. The dotted line indicates percent proliferation of anti-CD39 treated sample. Significance was determined using 2-tailed Students T-test, *P<0.05.

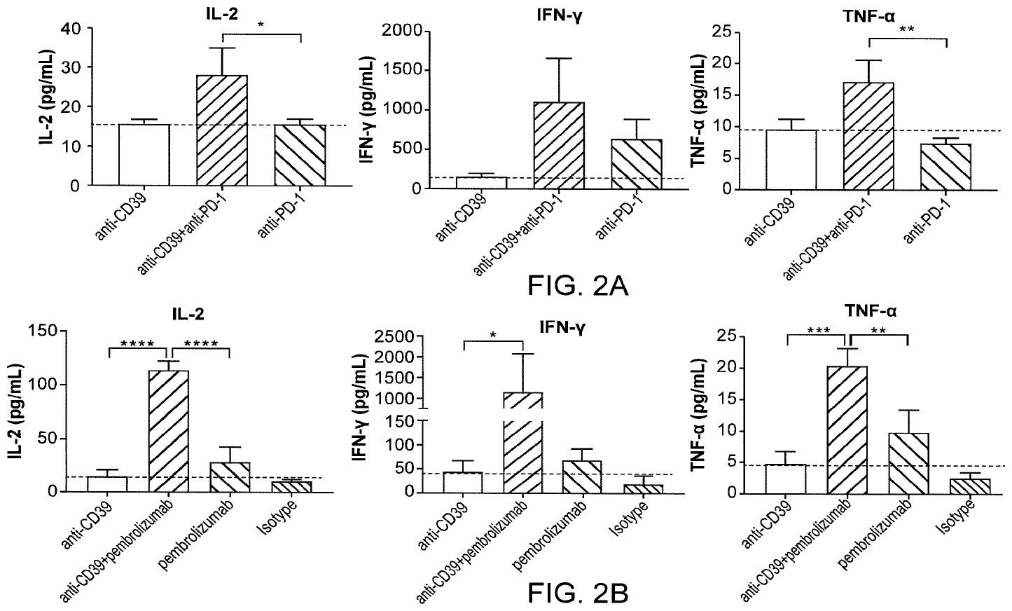

FIG. 2 shows that combined treatment of an anti-CD39 antibody and (A) an anti-PD-1 antibody enhances pro-inflammatory cytokine secretion in a one-way MLR. Cytokine type is depicted above each drawing. The x-axis depicts the amount of cytokine detected. Secretion of IL-2 (left panel), IFN-γ (center panel) and TNF-α was measured in the culture supernatants after 6 days of co-culture. The dotted line indicates cytokine concentration with anti-CD39 treatment only after 6 days co-culture. Significance was determined using 2-tailed Students T-test, significance was only tested for anti-PD-1 against the combination of anti-CD39 and anti-PD-1; *P<0.05, **P<0.01. In some instances (B), the anti-PD-1 antibody is pembrolizumab. Secretion of IL-2 (left panel), IFN-γ (center panel) and TNF-α was measured in the culture supernatants after 5 days of co-culture in the presence of 100 μM ATP. The dotted line indicates cytokine concentration with anti-CD39 treatment only after 5 days co-culture. Significance was determine using a 2-tailed Students T-test; The dotted line indicates cytokine concentration after 5 days co-culture. Significance was determined using 2-tailed Students T-test, significance was only tested for anti-PD-1 against the combination of anti-CD39 and anti-PD-1; *P<0.05, **P<0.01, ***P<0.001, ****P<0.00001.

According to FIG. 2 each of IL-2, IFN-γ, and TNF-∝ are enhanced with combined treatment of an anti-CD39 antibody and an anti-PD-1 antibody as opposed to either an anti-CD39 antibody and an anti-PD-1 alone. In one example, the anti-PD-1 antibody is pembrolizumab.

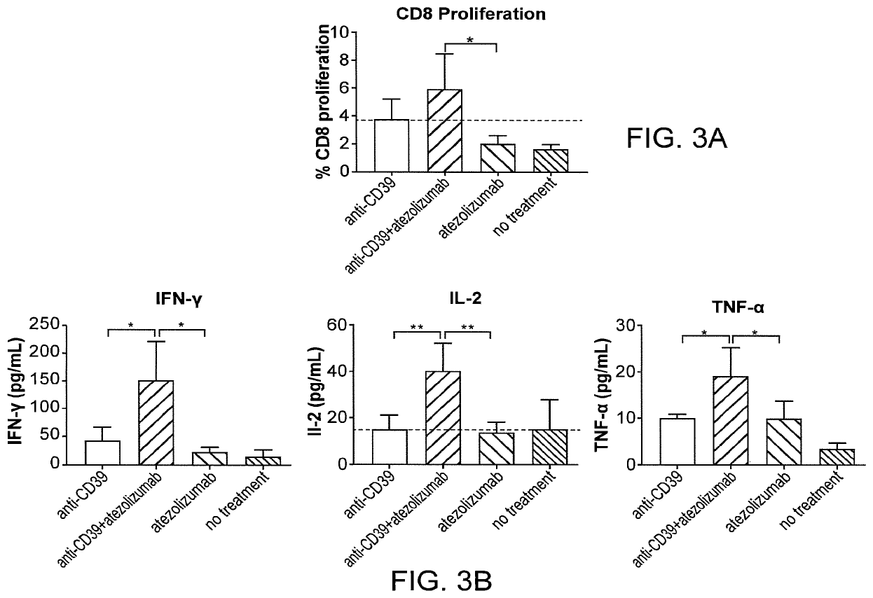

FIG. 3 shows combination treatment with an anti-CD39 antibody and an anti-PD-L1 antibody enhances (A) CD8⁺ T-cell proliferation and (B) pro-inflammatory cytokine production. CD8⁺ T-cell proliferation is depicted as the percentage of total CD8⁺ T cells that have undergone division. After 5 days of co-culture in the presence of 100 μM ATP, supernatants were collected and cytokines, including IL-2, IFN-γ, and TNF-∝, were measured. The dotted lines indicate the percent proliferation (A) or cytokine concentration (B) of cells treated with only anti-CD39. Significance was determined using 2-tailed Students T-test; *P<0.05, **P<0.01.

According to FIG. 3 CD8⁺ T-cell proliferation and each of IL-2, IFN-γ, and TNF-∝ are enhanced with combined treatment of an anti-CD39 antibody and an anti-PD-L1 antibody as opposed to either an anti-CD39 antibody and an anti-PD-1 alone. The anti-PD-L1 antibody may be atezolizumab.

### Example 3: Two-Way Mixed Lymphocyte Reaction

Whole PBMC from pairs of alloreactive donors were mixed and treated with 50 μg/ml anti-CD39 (HC SEQ ID NO: 255 and LC SEQ ID NO: 256) and/or anti-PD-1 (Pembrolizumab, Merck, Kenilworth, NJ, HC SEQ ID NO: 304 and LC SEQ ID NO: 305). Alternatively, the anti-CD39 antibody SRF360 with variable domain in hG4 format (HC SEC ID NO: 353 and LC SEQ ID NO: 354 ATP (Acros Organics, Geel, Belgium)) was added to all reactions, unless otherwise noted, for a final concentration of either 50 μM or 100 μM. Supernatants were harvested after 6 days and cytokines were measured using a Meso Scale Discovery human cytokine kit (MSD, Rockville, MD). Alternatively, an IL-2 Quantikine ELISA (R&D Systems, Minneapolis, MN) was used to quantitate IL-2.

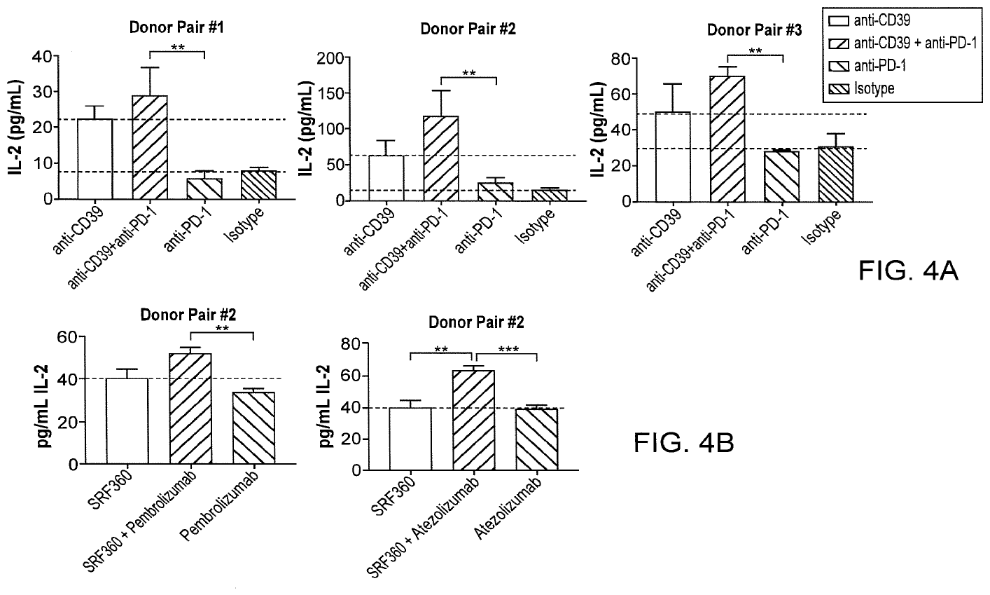

FIG. 4 (A) shows that combined treatment of an anti-CD39 antibody and an anti-PD-1 antibody enhances pro-inflammatory cytokine secretion in a two-way MLR. Each panel depicts a unique alloreactive donor pair. The x-axis depicts the type of antibody used and the y-axis depicts the amount of IL-2. The top dotted line depicts IL-2 secretion from anti-CD39 treated sample and the bottom dotted line depicts IL-2 secretion with an isotype-matched control antibody. Significance was determined using 2-tailed Students T-test, significance was only tested for anti-PD-1 against the combination of anti-CD39 and anti-PD-1; **P<0.01. (B) shows that combined treatment of an anti-CD39 antibody and an anti-PD-1 antibody enhances pro-inflammatory cytokine secretion in a two-way MLR when the anti-CD39 antibody is SRF360 and the anti-PD-1 antibody is pembrolizumab. The x-axis depicts the type of antibody used and the y-axis depicts the amount of IL-2. The dotted line depicts IL-2 secretion from anti-CD39 treated sample Significance was determined using 2-tailed Students T-test, significance was only tested for anti-PD-1 against the combination of anti-CD39 and anti-PD-1; **P<0.01, ***P<0.001.

### Example 4: Stimulated T Cell Assay

PBMCs were viably thawed and stained using CTV (ThermoFisher). PBMCs were activated with Immunocult (StemCell Technologies), which contains soluble tetrameric antibody complexes that bind CD3 and CD28 cell surface ligands. Activated cultures were treated with 25 μg/ml of an anti-CD39 antibody (HC SEQ ID NO: 255 and LC SEQ ID NO: 256) and/or 50 μg/ml of an anti-PD-1 antibody (Pembrolizumab, Merck, Kenilworth, NJ, HC SEQ ID NO: 304 and LC SEQ ID NO: 305). ATP (50 μM, Acros Organics) was added to all reactions except as indicated in the “No ATP” control. Supernatants were harvested after 5 days and cytokines were measured using a Meso Scale Discovery human cytokine kit (MSD, Rockville, MD). CD8⁺ T cells were discriminated using fluorescently labeled anti-CD3 and anti-CD8 antibodies (Biolegend, San Diego, CA); proliferation was calculated using the CTV dye dilution method by flow cytometry.

FIG. 4. shows that combined treatment of an anti-CD39 antibody and an anti-PD-1 antibody enhances T-cell proliferation and pro-inflammatory cytokine secretion from a stimulated PBMC. The x-axis depicts the type of antibody used and the absence of ATP. The y-axis on the left panel depicts % CD8⁺ proliferation and the y-axis on the right panel depicts amount of TNF-α. The top dotted line designates a no ATP control and the bottom dotted line designates isotype control. Significance was determined using 2-tailed Students T-test; **P<0.01.

An anti-CD39 antibody in combination with an anti-PD-1 antibody significantly increases proliferation of stimulated human CD8⁺ T cells and (B) secretion of TNF-α above anti-PD-1 treatment alone.

### Example 5: In-Vivo Studies Involving MC38

For the MC38 tumor study, C57BL/6 mice were injected subcutaneously in the flank with 5×10⁵ MC38 cells on Day 0. On day 4, mice received 250 μg of either isotype control (BioXcell, Clone MOPC-21) or an anti-CD39 antibody (Clone B66) intraperitoneally. On day 7 each group received an additional administration of the initial treatment and either 250 μg of anti-PD-L1 (BioXcell, Clone 10F.9G2) or an isotype control (BioXcell, Clone LTF-2) intraperitoneally. Mice were continued with the same regimen on days 11, 14, and 18.

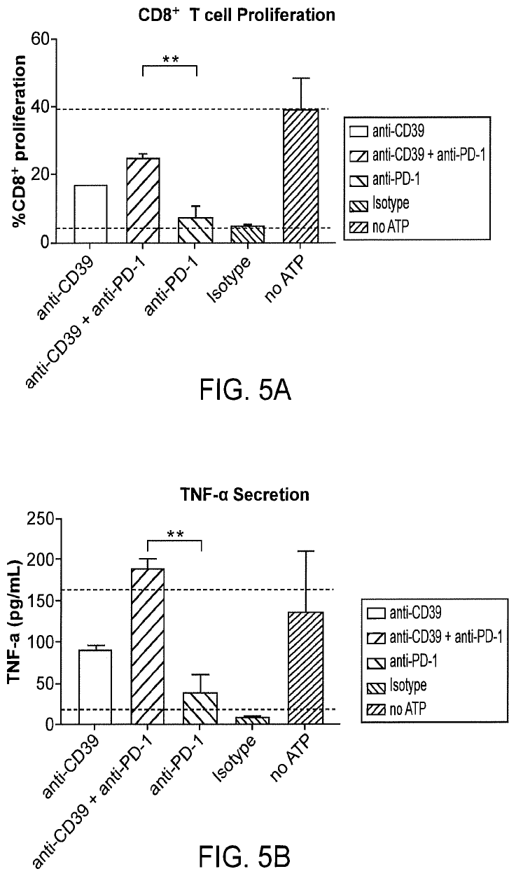

FIG. 5 shows that anti-CD39 in combination with an anti-PD-L1 therapy enhances anti-tumor responses in a MC38 syngeneic model. Curves depict mean tumor volume, with error bars indicated the SEM. Significance was determined using a One Way ANOVA with Dunn's multiple comparisons test; *P<0.05. The vertical dashed lines indicate drug combination dosing on days 8, 12, 15, and 19. The x-axis shows days after implantation and the y-axis show tumor volume.

For the CT26 tumor study, BALB/c mice were injected subcutaneously with 1×10⁵ CT26 cells on Day 0. On day 5, mice were treated intraperitoneally with 250 μg of either isotype (BioXcell, Clone MOPC-21) or anti-CD39 (Clone B66). On day 8, each group was further randomized and treated intraperitoneally with the initial day 5 treatment and either 250 μg of anti-PD-1 (BioXcell, Clone RMP1-14) or isotype control (BioXcell, Clone 2A3). Subsequently, all mice were continued on study the assigned combination therapy on days 12, 15, and 19.

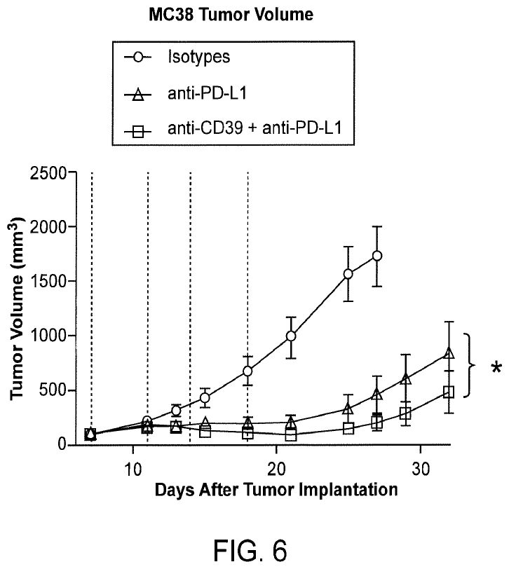

FIG. 6 shows that an anti-CD39 antibody demonstrates single agent activity and combinatorial effects with an anti-PD-1 antibody in the CT26 syngeneic tumor model. The combination of anti-CD39 and anti-PD-1 increases the number of complete responses compared to either monotherapy alone. The number of complete responses seen (out of n=10 animals per treatment group) are indicated for each treatment to the right of the curves. Curves depict mean tumor volume with error bars indicated the SEM. The vertical dashed lines indicate drug combination dosing on days 8, 12, 15, and 19. The x-axis shows days after implantation and the y-axis show tumor volume.

Animals with complete regressions following anti-CD39 treatment or anti-CD39 plus anti-PD-1 combination treatment were re-challenged subcutaneously with 1×10⁵ CT26 cells on day 49 following the original tumor challenge (indicated by the dashed vertical line). Complete responses were defined as animals that had a palpable tumor at randomization time, which later became undetectable. Tumor naive animals served as a control and tumor growth was measured until control animals had to be removed from the study due to tumor progression.

Tumor volume was assessed using the formula TV=0.5×(L×W²) where length (L) is the longest dimension of the tumor and width (W) is the longest dimension perpendicular to the length. Statistical significance of treatment was assessed using Prism V8.0 software (GraphPad Software, San Diego, CA).

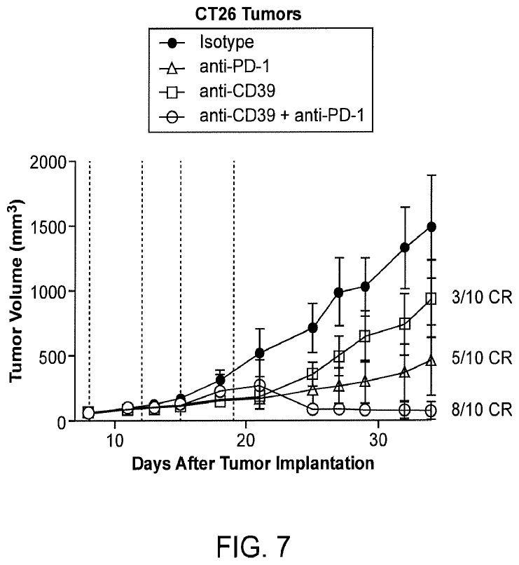

FIG. 7 shows that animals with complete responses following monotherapy with an anti-CD39 antibody and combination therapy with an anti-CD39 antibody and an anti-PD-1 antibody were resistant to tumor challenge. Curves depict mean tumor volume for animals that were rechallenged only: 5 tumor naïve animals (starting at day 49), n=3 animals with prior anti-CD39 antibody treatment and n=8 animals with prior anti-CD39 and anti-PD-1 combination treatment. Error bars indicated the SEM. The x-axis shows days after implantation and the y-axis show tumor volume.

### Example S: Sequences

Table S provides sequences referred to herein.

TABLE S
Sequences.

| SEQ | | | |
|---|---|---|---|
| ID NO: | Region | Binds | Sequence |
| 1 | CDR-H1 | CD39 | SYYMH |
| 2 | CDR-H1 | CD39 | SYEMH |
| 3 | CDR-H1 | CD39 | SYQMH |
| 4 | CDR-H1 | CD39 | SYYMY |
| 5 | CDR-H1 | CD39 | SYFMH |
| 6 | CDR-H1 | CD39 | SLAIS |
| 7 | CDR-H1 | CD39 | KLAIS |
| 8 | CDR-H1 | CD39 | HTAIS |
| 9 | CDR-H1 | CD39 | SLPIS |
| 10 | CDR-H1 | CD39 | LLAIS |
| 11 | CDR-H1 | CD39 | SNAIS |
| 12 | CDR-H1 | CD39 | AMAIS |
| 13 | CDR-H1 | CD39 | WLAIS |
| 14 | CDR-H1 | CD39 | SYAIS |
| 15 | CDR-H1 | CD39 | SYGIS |
| 16 | CDR-H1 | CD39 | KYGIS |
| 17 | CDR-H1 | CD39 | NYAIS |
| 18 | CDR-H1 | CD39 | SYATS |
| 19 | CDR-H1 | CD39 | SYAIG |
| 20 | CDR-H1 | CD39 | SYSMN |
| 21 | CDR-H1 | CD39 | SYGMN |
| 22 | | | |
| 23 | | | |
| 24 | | | |
| 25 | CDR-H1 | PD-1 (181) | HYGMN |
| 26 | CDR-H1 | PD-1 (Nivolumab) | NSGMH |
| 27 | CDR-H1 | PD-1 | NYYMY |
| | | (Pembrolizumab) | |
| 28 | CDR-H1 | PD-1 | NFGMT |
| | | (Cemiplimab) | |
| 29 | CDR-H1 | PD-L1 | DSWIH |
| | | (Atezolizumab) | |
| 30 | CDR-H1 | PD-L1 | SYIMM |
| | | (Avelumab) | |
| 31 | CDR-H1 | PD-L1 | RYWMS |
| | | (Durvalumab) | |
| 32 | CDR-H2 | CD39 | VINPSGGSTSYAQKFQG |
| 33 | CDR-H2 | CD39 | RINPSVGSTWYAQKFQG |
| 34 | CDR-H2 | CD39 | RINPSGGSTWYAQKFQG |
| 35 | CDR-H2 | CD39 | KINPSGGSTWYAQKFQG |
| 36 | CDR-H2 | CD39 | VINPLGGGTSYAQKFQG |
| 37 | CDR-H2 | CD39 | SINPRGGSTSYAQKFQG |
| 38 | CDR-H2 | CD39 | GIIPIFGTANYAQKFQG |
| 39 | CDR-H2 | CD39 | GI - - GFGTANYAQKFQG |
| 40 | CDR-H2 | CD39 | GILPIGGTANYAQKFQG |
| 41 | CDR-H2 | CD39 | GILPIAGTANYAQKFQG |
| 42 | CDR-H2 | CD39 | GILPIFGEANYAQKFQG |
| 43 | CDR-H2 | CD39 | GIIPRGGTANYAQKFQG |
| 44 | CDR-H2 | CD39 | SIIPIFGTANYAQKFRG |
| 45 | CDR-H2 | CD39 | SIIPEFGIANYAQKFQG |
| 46 | CDR-H2 | CD39 | SIIPIFGTANYAQKFQG |
| 47 | CDR-H2 | CD39 | GIIPISGTANYAQEFQG |
| 48 | CDR-H2 | CD39 | GIIPTFGTANYAQKFQG |
| 49 | CDR-H2 | CD39 | SISSSSSYIYYADSVKG |
| 50 | CDR-H2 | CD39 | VIWYDGSNKYYADSVKG |
| 51 | CDR-H2 | PD-1 (181) | WVNTYTGEPTYADDFKG |
| 52 | CDR-H2 | PD-1 (Nivolumab) | VIWYDGSKRYYADSVKG |
| 53 | CDR-H2 | PD-1 | GINPSNGGTNFNEKFKN |
| | | (Pembrolizumab) | |
| 54 | CDR-H2 | PD-1 | GISGGGRDTYFADSVKG |
| | | (Cemiplimab) | |
| 55 | CDR-H2 | PD-L1 | WISPYGGSTYYADSVKG |
| | | (Atezolizumab) | |
| 56 | CDR-H2 | PD-L1 | SIYPSGGITFYADTVKG |
| | | (Avelumab) | |
| 57 | CDR-H2 | PD-L1 | NIKQDGSEKYYVDSVKG |
| | | (Durvalumab) | |
| 58 | CDR-H3 | CD39 | GKREGGTEYLRH |
| 59 | CDR-H3 | CD39 | GKREGGTEYLRK |
| 60 | CDR-H3 | CD39 | GKREGGTEYLRS |
| 61 | CDR-H3 | CD39 | GKREGGTEYLRN |
| 62 | CDR-H3 | CD39 | GKREGGTEYLRV |
| 63 | CDR-H3 | CD39 | GGAKYASTYGMDV |
| 64 | CDR-H3 | CD39 | GGAKYASTHGMDV |
| 65 | CDR-H3 | CD39 | GGAKYASQLGMDV |
| 66 | CDR-H3 | CD39 | GGAKYASKWGMDV |
| 67 | CDR-H3 | CD39 | GGAKYAVGYGMDV |
| 68 | CDR-H3 | CD39 | GGAKYAGRYGMDV |
| 69 | CDR-H3 | CD39 | GGAKYARTYGMDV |
| 70 | CDR-H3 | CD39 | ESGGYRDHRLDV |
| 71 | CDR-H3 | CD39 | ESGTYRDHRLDV |
| 72 | CDR-H3 | CD39 | ESGGYRDHRLGV |
| 73 | CDR-H3 | CD39 | DFTDYSSGYSSGWTY |
| 74 | CDR-H3 | CD39 | DTLYSSGAYYGYNV |
| 75 | CDR-H3 | CD39 | AKRGYDSYGGVYFDY |
| 76 | CDR-H3 | CD39 | GPTVTATTSIGTHNWFDP |
| 77 | CDR-H3 | CD39 | EGRGYDSSRYYKFWFDPWGQGTLVTVSS |
| 78 | CDR-H3 | CD39 | DGGGYRHHYFDL |
| 79 | CDR-H3 | CD39 | ESGGYRDHKLDV |
| 80 | CDR-H3 | CD39 | DGGGYQHHYFDL |
| 81 | CDR-H3 | CD39 | DSGYHRHYSDY |
| 82 | CDR-H3 | CD39 | DPLGIRKHWFDP |
| 83 | CDR-H3 | CD39 | DTPRWRYHYFDY |
| 84 | CDR-H3 | CD39 | ERRGSLALGMDV |
| 85 | CDR-H3 | CD39 | DLGGYSYGEPYYYYYGMDV |
| 86 | CDR-H3 | PD-1 (181) | EGEGLGFGD |
| 87 | CDR-H3 | PD-1 (Nivolumab) | NDDY |
| 88 | CDR-H3 | PD-1 | RDYRFDMGFDY |
| | | (Pembrolizumab) | |
| 89 | CDR-H3 | PD-1 | WGNIYFDY |
| | | (Cemiplimab) | |
| 90 | CDR-H3 | PD-L1 | RHWPGGFDY |
| | | (Atezolizumab) | |
| 91 | CDR-H3 | PD-L1 | IKLGTVTTVDY |
| | | (Avelumab) | |
| 92 | CDR-H3 | PD-L1 | EGGWFGELAFDY |
| | | (Durvalumab) | |
| 93 | CDR-L1 | CD39 | RASQSVSSSYLA |
| 94 | CDR-L1 | CD39 | RASQSVASSYLA |
| 95 | CDR-L1 | CD39 | EASQSVSYSYLA |
| 96 | CDR-L1 | CD39 | KASESVSSSYLA |
| 97 | CDR-L1 | CD39 | RASQYVSSSYLA |
| 98 | CDR-L1 | CD39 | KSSQSVLFSSNNKNYLA |
| 99 | CDR-L1 | CD39 | KSSRSVLFSSNNKNYLA |
| 100 | CDR-L1 | CD39 | KSSKSVLYSNNNKNYLA |
| 101 | CDR-L1 | CD39 | RASQSVGSNLA |
| 102 | CDR-L1 | CD39 | KSSQSVLYSSNNKNYLA |
| 103 | CDR-L1 | CD39 | QASQDISNYLN |
| 104 | CDR-L1 | CD39 | RASQSVSSYLA |
| 105 | CDR-L1 | CD39 | RASQSVSRYLA |
| 106 | CDR-L1 | CD39 | RASQSISSWLA |
| 107 | CDR-L1 | CD39 | RASQSVSSDYLA |
| 108 | CDR-L1 | PD-1 (181) | RSSQSIVHSHGDTYLE |
| 109 | CDR-L1 | PD-1 (Nivolumab) | RASQSVSSYLA |
| 110 | CDR-L1 | PD-1 | RASKGVSTSGYSYLH |
| | | (Pembrolizumab) | |
| 111 | CDR-L1 | PD-1 | RASLSINTFLN |
| | | (Cemiplimab) | |
| 112 | CDR-L1 | PD-L1 | RASQDVSTAVA |
| | | (Atezolizumab) | |
| 113 | CDR-L1 | PD-L1 | TGTSSDVGGYNYVS |
| | | (Avelumab) | |
| 114 | CDR-L1 | PD-L1 | RASQRVSSSYLA |
| | | (Durvalumab) | |
| 115 | CDR-L2 | CD39 | GASSRAT |
| 116 | CDR-L2 | CD39 | GASNRHT |
| 117 | CDR-L2 | CD39 | YASSRAY |
| 118 | CDR-L2 | CD39 | GASSRAN |
| 119 | CDR-L2 | CD39 | YASSRAT |
| 120 | CDR-L2 | CD39 | YASNRAT |
| 121 | CDR-L2 | CD39 | WASTRES |
| 122 | CDR-L2 | CD39 | WASSRES |
| 123 | CDR-L2 | CD39 | WASTRQS |
| 124 | CDR-L2 | CD39 | WASTRAS |
| 125 | CDR-L2 | CD39 | GASTRAT |
| 126 | CDR-L2 | CD39 | GASTRAS |
| 127 | CDR-L2 | CD39 | DASNLET |
| 128 | CDR-L2 | CD39 | DASNRAT |
| 129 | CDR-L2 | CD39 | DASKRAT |
| 130 | CDR-L2 | CD39 | KASSLES |
| 131 | CDR-L2 | PD-1 (181) | KVSNRFS |
| 132 | CDR-L2 | PD-1 (Nivolumab) | DASNRAT |
| 133 | CDR-L2 | PD-1 | LASYLES |
| | | (Pembrolizumab) | |
| 134 | CDR-L2 | PD-1 | AASSLHG |
| | | (Cemiplimab) | |
| 135 | CDR-L2 | PD-L1 | SASFLYS |
| | | (Atezolizumab) | |
| 136 | CDR-L2 | PD-L1 | DVSNRPS |
| | | (Avelumab) | |
| 137 | CDR-L2 | PD-L1 | DASSRAT |
| | | (Durvalumab) | |
| 138 | CDR-L3 | CD39 | QQYHSYIT |
| 139 | CDR-L3 | CD39 | QQYHNAIT |
| 140 | CDR-L3 | CD39 | QQYYFYIT |
| 141 | CDR-L3 | CD39 | QQYHSALT |
| 142 | CDR-L3 | CD39 | QQYHGGIT |
| 143 | CDR-L3 | CD39 | QQYHRRIT |
| 144 | CDR-L3 | CD39 | QQYHSGIT |
| 145 | CDR-L3 | CD39 | QQYYLYPLT |
| 146 | CDR-L3 | CD39 | QQYWTYPLT |
| 147 | CDR-L3 | CD39 | QQYLLYPLT |
| 148 | CDR-L3 | CD39 | QQYLIWPLT |
| 149 | CDR-L3 | CD39 | QQYLLWPLT |
| 150 | CDR-L3 | CD39 | QQFYFFPPT |
| 151 | CDR-L3 | CD39 | QQAYTFPPT |
| 152 | CDR-L3 | CD39 | QQYYIFPPT |
| 153 | CDR-L3 | CD39 | QQRNFYPPT |
| 154 | CDR-L3 | CD39 | QQFVLWPRT |
| 155 | CDR-L3 | CD39 | QQHVNFPLT |
| 156 | CDR-L3 | CD39 | QQSVFWPIT |
| 157 | CDR-L3 | CD39 | QQLTKWPLT |
| 158 | CDR-L3 | CD39 | QQDVLWPLT |
| 159 | CDR-L3 | CD39 | QQYGLFPIT |
| 160 | CDR-L3 | CD39 | QQHTVWPIT |
| 161 | CDR-L3 | CD39 | QQVLNYPLT |
| 162 | CDR-L3 | CD39 | QQSYFLPPT |
| 163 | CDR-L3 | CD39 | QQAHSSPYT |
| 164 | CDR-L3 | PD-1 (181) | FQGSHIPVT |
| 165 | CDR-L3 | PD-1 (Nivolumab) | QQSSNWPRT |
| 166 | CDR-L3 | PD-1 | QHSRDLPLT |
| | | (Pembrolizumab) | |
| 167 | CDR-L3 | PD-1 | QQSSNTPFT |
| | | (Cemiplimab) | |
| 168 | CDR-L3 | PD-L1 | QQYLYHPAT |
| | | (Atezolizumab) | |
| 169 | CDR-L3 | PD-L1 | SSYTSSSTRV |
| | | (Avelumab) | |
| 170 | CDR-L3 | PD-L1 | QQYGSLPWT |
| | | (Durvalumab) | |
| 171 | VH | CD39 | QVQLVQSGAEVKEPGASVKVSCKAPGYT |
| | | | FTSYYMHWVRQAPGQGLEWMGVINPSGG |
| | | | STSYAQKFQGRVTMTRDTSTSTVYMELS |
| | | | SLRSEDTAVYYCARGKREGGTEYLRHWG |
| | | | QGTLVTVSS |
| 172 | VH | CD39 | QVQLVQSGAEVKKPGASVKVSCKASGYT |
| | | | FKSYEMHWVRQAPGQGLEWMGRINPSVG |
| | | | STWYAQKFQGRVTMTRDTSTSTVYMELS |
| | | | SLRSEDTAVYYCARGKREGGTEYLRKWG |
| | | | QGTLVTVSS |
| 173 | VH | CD39 | QVQLVQSGAEVKKPGASVKVSCKASGYT |
| | | | FTSYQMHWVRQAPGQGLEWMGRINPSGG |
| | | | STWYAQKFQGRVTMTRDTSTSTVYMELS |
| | | | SLRSEDTAVYYCARGKREGGTEYLRSWG |
| | | | QGTLVTVSS |
| 174 | VH | CD39 | QVQLVQSGAEVKKPGASVKVSCKASGYT |
| | | | FKSYEMHWVRQAPGQGLEWMGRINPSVG |
| | | | STWYAQKFQGRVTMTRDTSTSTVYMELS |
| | | | SLRSEDTAVYYCARGKREGGTEYLRNWG |
| | | | QGTLVTVSS |
| 175 | VH | CD39 | QVQLVQSGAEVKKPGASVKVSCKASGYI |
| | | | FKSYEMHWVRQAPGQGLEWMGRINPSVG |
| | | | STWYAQKFQGRVTMTRDTSTSTVYMELS |
| | | | SLRSEDTAVYYCARGKREGGTEYLRVWG |
| | | | QGTLVTVSS |
| 176 | VH | CD39 | QVQLVQSGAEVKKPGASVKVSCKASGYT |
| | | | FQSYYMHWVRQAPGQGLEWMGKINPSGG |
| | | | STWYAQKFQGRVTMTRDTSTSTVYMELS |
| | | | SLRSEDTAVYYCARGKREGGTEYLRHWG |
| | | | QGTLVTVSS |
| 177 | VH | CD39 | QVQLVQSGAEVKKPGASVKVSCKASGYT |
| | | | FKSYEMHWVRQAPGQGLEWMGRINPSGG |
| | | | STWYAQKFQGRVTMTRDTSTSTVYMELS |
| | | | SLRSEDTAVYYCARGKREGGTEYLRHWG |
| | | | QGTLVTVSS |
| 178 | VH | CD39 | QVQLVQSGAEVKKPGASVKVSCKASGYT |
| | | | FTSYQMHWVRQAPGQGLEWMGRINPSGG |
| | | | STWYAQKFQGRVTMTRDTSTSTVYMELS |
| | | | SLRSEDTAVYYCARGKREGGTEYLRHWG |
| | | | QGTLVTVSS |
| 179 | VH | CD39 | QVQLVQSGAEVKKPGASVKVSCKASGYT |
| | | | FFSYYMYWVRQAPGQGLEWMGVINPLGG |
| | | | GTSYAQKFQGRVTMTRDTSTSTVYMELS |
| | | | SLRSEDTAVYYCARGKREGGTEYLRHWG |
| | | | QGTLVTVSS |
| 180 | VH | CD39 | QVQLVQSGAEVKKPGASVKVSCKASGYT |
| | | | FVSYFMHWVRQAPGQGLEWMGSINPRGG |
| | | | STSYAQKFQGRVTMTRDTSTSTVYMELS |
| | | | SLRSEDTAVYYCARGKREGGTEYLRHWG |
| | | | QGTLVTVSS |
| 181 | VH | CD39 | QVQLVQSGAEVKKPGASVKVSCKASGYT |
| | | | FKSYEMHWVRQAPGQGLEWMGRINPSVG |
| | | | STWYAQKFQGRVTMTRDTSTSTVYMELS |
| | | | SLRSEDTAVYYCARGKREGGTEYLRHWG |
| | | | QGTLVTVSS |
| 182 | VH | CD39 | QVQLVQSGAEVKKPGSSVKVSCKASGGT |
| | | | FSSLAISWVRQAPGQGLEWMGGIIPIFG |
| | | | TANYAQKFQGRVTITADESTNTAYMELS |
| | | | SLRSEDTAVYYCARGGAKYASTYGMDVW |
| | | | GQGTTVTVSS |
| 183 | VH | CD39 | QVQLVQSGAEVKKPGSSVKVSCKASGGT |
| | | | FSKLAISWVRQAPGQGLEWMGGIGFGTA |
| | | | NYAQKFQGRVTITADESASTAYMELSSL |
| | | | RSEDTAVYYCARGGAKYASTHGMDVWGQ |
| | | | GTTVTVSS |
| 184 | VH | CD39 | QVQLVQSGAEVKKPGSSVKVSCKASGGT |
| | | | FSHTAISWVRQAPGQGLEWMGGILPIGG |
| | | | TANYAQKFQGRVTITADESTSTAYMELS |
| | | | SLRSEDTAVYYCARGGAKYASQLGMDVW |
| | | | GQGTTVTVSS |
| 185 | VH | CD39 | QVQLVQSGAEVKKPGSSVKVSCKASGGT |
| | | | FSSLPISWVRQAPGQGLEWMGGIGFGTA |
| | | | NYAQKFQGRVTITADESTSTAYMELSSL |
| | | | RSEDTAVYYCARGGAKYASKWGMDVWGQ |
| | | | GTTVTVSS |
| 186 | VH | CD39 | QVQLVQSGAEVKKPGSSVKVSCKASGGT |
| | | | FSLLAISWVRQAPGQGLEWMGGILPIAG |
| | | | TANYAQKFQGRVTITADESTSTAYMELS |
| | | | SLRSEDTAVYYCARGGAKYAVGYGMDVW |
| | | | GQGTTVTVSS |
| 187 | VH | CD39 | QVQLVQSGAEVKKPGASVKVSCKASGGT |
| | | | FQSLAISWVRQAPGQGLEWMGGILPIGG |
| | | | TANYAQKFQGRVTITADESTSTAYMELS |
| | | | SLRSEDTAVYYCARGGAKYAGRYGMDVW |
| | | | GQGTTVTVSS |
| 188 | VH | CD39 | QVQLVQSGAEVKKPGSSVKVSCKASGGT |
| | | | FPSNAISWVRQAPGQGLEWMGGIGFGTA |
| | | | NYAQKFQGRVTITADESTSTAYMELSSL |
| | | | RSEDTAVYYCARGGAKYARTYGMDVWGQ |
| | | | GTTVTVSS |
| 189 | VH | CD39 | QVQLVQSGAEVKKPGSSVKVSCKASGGT |
| | | | FSSLPISWVRQAPGQGLEWMGGIGFGTA |
| | | | NYAQKFQGRVTITADESTSTAYMELSSL |
| | | | RSEDTAVYYCARGGAKYAGRYGMDVWGQ |
| | | | GTTVTVSS |
| 190 | VH | CD39 | QVQLVQSGAEVKKPGSSVKVSCKASGGT |
| | | | FSAMAISWVRQAPGQGLEWMGGILPIAG |
| | | | TANYAQKFQGRVTITADESTSTAYMELS |
| | | | SLRSEDTAVYYCARGGAKYASTYGMDVW |
| | | | GQGTTVTVSS |
| 191 | VH | CD39 | QVQLVQSGAEVKKPGSSVKVSCKASGGT |
| | | | FASLAISWVRQAPGQGLEWMGGILPIFG |
| | | | EANYAQKFQGRVTITADESTSTAYMELS |
| | | | SLRSEDTAVYYCARGGAKYASTYGMDVW |
| | | | GQGTTVTVSS |
| 192 | VH | CD39 | QVQLVQSGAEVKKPGSSVKVSCKASGGT |
| | | | FSWLAISWVRQAPGQGLEWMGGIIPRGG |
| | | | TANYAQKFQGRVTITADESTSTAYMELS |
| | | | SLRSEDTAVYYCARGGAKYASTYGMDVW |
| | | | GQGTTVTVSS |
| 193 | VH | CD39 | QVQLVQSGAEVKKPGSSVKASCKASGGT |
| | | | FSSYAISWVRQAPGQGLEWMGSIIPIFG |
| | | | TANYAQKFRGRVTITADESTSTTYMELS |
| | | | SLRSEDTAVYYCARESGGYRDHRLDVWG |
| | | | QGTMVTVSS |
| 194 | VH | CD39 | QVQLVQSGAEVKKPGSSVKVSCKASGGT |
| | | | FGSYGISWVRQAPGQGLEWMGSIIPEFG |
| | | | IANYAQKFQGRVTITADESTSTAYMELS |
| | | | SLRSEDTAVYYCARESGTYRDHRLDVWG |
| | | | QGTMVTVSS |
| 195 | VH | CD39 | QVQLVQSGAEVKKPGSSVKVSCKASGGT |
| | | | FSKYGISWVRQAPGQGLEWMGSIIPEFG |
| | | | IANYAQKFQGRVTITADESTSTAYMELS |
| | | | SLRSEDTAVYYCARESGGYRDHRLGVWG |
| | | | QGTMVTVSS |
| 196 | VH | CD39 | QVQLVQSGAEVKKPGSSVKVSCKASGGT |
| | | | FESYGISWVRQAPGQGLEWMGSIIPEFG |
| | | | IANYAQKFQGRVTITADESTSTTYMELS |
| | | | SLRSEDTAVYYCARESGGYRDHRLDVWG |
| | | | QGTMVTVSS |
| 197 | VH | CD39 | QVQLVQSGAEVKKPGSSVKVSCKASGGT |
| | | | FSSYAISWVRQAPGQGLEWMGGIIPIFG |
| | | | TANYAQKFQGRVTITADESTSTAYMELS |
| | | | SLRSEDTAVYYCARDFTDYSSGYSSGWT |
| | | | YWGQGTLVTVSS |
| 198 | VH | CD39 | QVQLVQSGAEVKKPGSSVKVSCKASGGT |
| | | | FSNYAISWVRQAPGQGLEWMGGIIPIFG |
| | | | TANYAQKFQGRVTITADESTSTAYMELS |
| | | | SLRSEDTAVYYCARDTLYSSGAYYGYNV |
| | | | WGQGTMVTVSS |
| 199 | VH | CD39 | QVQLVQSGAEVKKPGSSVKVSCKASGGT |
| | | | FSNYAISWVRQAPGQGLEWMGGIIPIFG |
| | | | TANYAQKFQGRVTITADESTSTAYMELS |
| | | | SLRSEDTAVYYCARAKRGYDSYGGVYFD |
| | | | YWGQGTLVTVSS |
| 200 | VH | CD39 | QVQLVQSGAEVKKPGSSVKVSCKASGGT |
| | | | FSNYAISWVRQAPGQGLEWMGGIIPIFG |
| | | | TANYAQKFQGRVTITADESTSTAYMELS |
| | | | SLRSEDTAVYYCARGPTVTATTSIGTHN |
| | | | WFDPWGQGTLVTVSS |
| 201 | VH | CD39 | QVQLVQSGAEVKKPGSSVKVSCKASGGT |
| | | | FSSYAISWVRQAPGQGLEWMGSIIPIFG |
| | | | TANYAQKFQGRVTITADESTSTAYMELS |
| | | | SLRSEDTAVYYCAREGRGYDSSRYYKFW |
| | | | FDPWGQGTLVTVSS |
| 202 | VH | CD39 | QVQLVQSGAEVKEPGSSVKVSCKASGGT |
| | | | FSSYATSWVRQAPGQGLEWMGGIIPISG |
| | | | TANYAQEFQGRVTITADESTSTAYMELS |
| | | | SLRSEDTAVYYCARDGGGYRHHYFDLWG |
| | | | RGTLVTVSS |
| 203 | VH | CD39 | QVQLVQSGAEVKKPGSSVKVPCKASGGT |
| | | | FSSYAISWVRQAPEQGLEWMGSIIPIFG |
| | | | TANYAQKFQGRVTITADESTSTAYMELS |
| | | | SLRSEDTAVYYCAGESGGYRDHKLDVWG |
| | | | QGTVVTVSS |
| 204 | VH | CD39 | QVQLVQSGAEVKKPGSSVKVSCKASGGA |
| | | | FSSYAIGWVRQAPGQGLEWMGGIIPTFG |
| | | | TANYAQKFQGRVTITADESTSTAYMELS |
| | | | SLRSEDTAVYYCARDGGGYQHHYFDLWG |
| | | | RGTLVTVSS |
| 205 | VH | CD39 | QVQLVQSGAEVKKPGSSVKVSCKASGGT |
| | | | FSSYAISWVRQAPGQGLEWMGSIIPIFG |
| | | | TANYAQKFQGRVTITADESTSTAYMELS |
| | | | SLRSEDTAVYYCARESGGYRDHKLDVWG |
| | | | QGTMVTVSS |
| 206 | VH | CD39 | QVQLVQSGAEVKKPGSSVKVSCKASGGT |
| | | | FSSYAISWVRQAPGQGLEWMGGIIPIFG |
| | | | TANYAQKFQGRVTITADESTSTAYMELS |
| | | | SLRSEDTAVYYCARDSGYHRHYSDYWGQ |
| | | | GTLVTVSS |
| 207 | VH | CD39 | QVQLVQSGAEVKKPGSSVKVSCKASGGT |
| | | | FSSYAISWVRQAPGQGLEWMGGIIPIFG |
| | | | TANYAQKFQGRVTITADESTSTAYMELS |
| | | | SLRSEDTAVYYCARDPLGIRKHWFDPWG |
| | | | QGTLVTVSS |
| 208 | VH | CD39 | QVQLVQSGAEVKKPGSSVKVSCKASGGT |
| | | | FSSYAISWVRQAPGQGLEWMGGIIPIFG |
| | | | TANYAQKFQGRVTITADESTSTAYMELS |
| | | | SLRSEDTAVYYCARDTPRWRYHYFDYWG |
| | | | QGTLVTVSS |
| 209 | VH | CD39 | EVQLVESGGGLVKPGGSLRLSCAASGFT |
| | | | FSSYSMNWVRQAPGKGLEWVSSISSSSS |
| | | | YIYYADSVKGRFTISRDNAKNSLYLQMN |
| | | | SLRAEDTAVYYCARERRGSLALGMDVWG |
| | | | QGTLVTVSS |
| 210 | VH | CD39 | QVQLVESGGGWQPGRSLRLSCAASGFT |
| | | | FSSYGMNWVRQAPGKGLEWVAVIWYDGS |
| | | | NKYYADSVKGRFTISRDNSKNTLYLQMN |
| | | | SLRAEDTAVYYCARDLGGYSYGEPYYYY |
| | | | YGMDVWGQGTTVTVSS |
| 211 | VH | PD-1(181) | EIQLVQSGAEVKKPGSSVKVSCKASGYT |
| | | | FTHYGMNWVRQAPGQGLEWVGvNNTYTG |
| | | | EPTYADDFKGRLTFTLDTSTSTAYMELS |
| | | | SLRSEDTAVYYCTREGEGLGFGDWGQGT |
| | | | TVTVSS |
| 212 | VH | PD-1 (Nivolumab) | QVQLVESGGGWQPGRSLRLDCKASGIT |
| | | | FSNSGMHWVRQAPGKGLEWVAVIWYDGS |
| | | | KRYYADSVKGRFTISRDNSKNTLFLQMN |
| | | | SLRAEDTAVYYCATNDDYWGQGTLVTVS |
| | | | S |
| 213 | VH | PD-1 | QVQLVQSGVEVKKPGASVKVSCKASGYT |
| | | (Pembrolizumab) | FTNYYMYWVRQAPGQGLEWMGGINPSNG |
| | | | GTNFNEKFKNRVTLTTDSSTTTAYMELK |
| | | | SLQFDDTAVYYCARRDYRFDMGFDYWGQ |
| | | | GTTVTVSS |
| 214 | VH | PD-1 | EVQLLESGGVLVQPGGSLRLSCAASGFT |
| | | (Cemiplimab) | FSNFGMTWVRQAPGKGLEWVSGISGGGR |
| | | | DTYFADSVKGRFTISRDNSKNTLYLQMN |
| | | | SLKGEDTAVYYCVKWGNIYFDYWGQGTL |
| | | | VTVSS |
| 215 | VH | PD-L1 | EVQLVESGGGLVQPGGSLRLSCAASGFT |
| | | (Atezolizumab) | FSDSWIHWVRQAPGKGLEWVAWISPYGG |
| | | | STYYADSVKGRFTISADTSKNTAYLQMN |
| | | | SLRAEDTAVYYCARRHWPGGFDYWGQGT |
| | | | LVTVSS |
| 216 | VH | PD-L1 | EVQLLESGGGLVQPGGSLRLSCAASGFT |
| | | (Avelumab) | FSSYIMMWVRQAPGKGLEWVSSIYPSGG |
| | | | ITFYADTVKGRFTISRDNSKNTLYLQMN |
| | | | SLRAEDTAVYYCARIKLGTVTTVDYWGQ |
| | | | GTLVTVSS |
| 217 | VH | PD-L1 | EVQLVESGGGLVQPGGSLRLSCAASGFT |
| | | (Durvalumab) | FSRYWMSWVRQAPGKGLEWVANIKQDGS |
| | | | EKYYVDSVKGRFTISRDNAKNSLYLQMN |
| | | | SLRAEDTAVYYCAREGGWFGELAFDYWG |
| | | | QGTLVTVSS |
| 218 | VL | CD39 | EIVLTQSPGTLSLSPGERATLSCRASQS |
| | | | VSSSYLAWYQQKPGQAPRLLIYGASSRA |
| | | | TGIPDRFSGSGSGTDFTLTISRLEPEDF |
| | | | AVYYCQQYHSYITFGGGTKVEIK |
| 219 | VL | CD39 | EIVLTQSPGTLSLSPGERATLSCRASQS |
| | | | VASSYLAWYQQKPGQAPRLLIYGASNRH |
| | | | TGIPDRFSGSGSGTDFTLTISRLEPEDF |
| | | | AVYYCQQYHNAITFGGGTKVEIK |
| 220 | VL | CD39 | EIVLTQSPGTLSLSPGERATLSCRASQS |
| | | | VSSSYLAWYQQKPGQAPRLLIYYASSRA |
| | | | YGIPDRFSGSGSGTDFTLTISRLEPEDF |
| | | | AVYYCQQYHNAITFGGGTKVEIK |
| 221 | VL | CD39 | EIVLTQSPGTLSLSPGERATLSCRASQS |
| | | | VSSSYLAWYQQKPGQAPRLLIYGASSRA |
| | | | TGIPDRFSGSGSGTDFTLTISRLEPEDF |
| | | | AVYYCQQYYFYITFGGGTKVEIK |
| 222 | VL | CD39 | EIVLTQSPGTLSLSPGERATLSCEASQS |
| | | | VSYSYLAWYQQKPGQAPRLLIYGASSRA |
| | | | NGIPDRFSGSGSGTDFTLTISRLEPEDF |
| | | | AVYYCQQYHSALTFGGGTKVEIK |
| 223 | VL | CD39 | EIVLTQSPGTLSLSPGERATLSCRASQS |
| | | | VASSYLAWYQQKPGQAPRLLIYGASNRH |
| | | | TGIPDRFSGSGSGTDFTLTISRLEPEDF |
| | | | AVYYCQQYHGGITFGGGTKVEIK |
| 224 | VL | CD39 | EIVLTQSPGTLSLSPGERATLSCKASES |
| | | | VSSSYLAWYQQKPGQAPRLLIYYASSRA |
| | | | TGIPDRFSGSGSGTDFTLTISRLEPEDF |
| | | | AVYYCQQYHRRITFGGGTKVEIK |
| 225 | VL | CD39 | EIVLTQSPGTLSLSPGERATLSCRASQY |
| | | | VSSSYLAWYQQKPGQAPRLLIYYASNRA |
| | | | TGIPDRFSGSGSGTDFTLTISRLEPEDF |
| | | | AVYYCQQYHSGITFGGGTKVEIK |
| 226 | VL | CD39 | DIVMTQSPDSLAVSLGERATINCKSSQS |
| | | | VLFSSNNKNYLAWYQQKPGQPPKLLIYW |
| | | | ASTRESGVPDRFSGSGSGTDFTLTISSL |
| | | | QAEDVAVYYCQQYYLYPLTFGGGTKVEI |
| | | | K |
| 227 | VL | CD39 | DIVMTQSPDSLAVSLGERATINCKSSRS |
| | | | VLFSSNNKNYLAWYQQKPGQPPKLLIYW |
| | | | ASTRESGVPDRFSGSGSGTDFTLTISSL |
| | | | QAEDVAVYYCQQYWTYPLTFGGGTKVEI |
| | | | K |
| 228 | VL | CD39 | DIVMTQSPDSLAVSLGERATINCKSSQS |
| | | | VLFSSNNKNYLAWYQQKPGQPPKLLIYW |
| | | | ASSRESGVPDRFSGSGSGTDFTLTISSL |
| | | | QAEDVAVYYCQQYWTYPLTFGGGTKVEI |
| | | | K |
| 229 | VL | CD39 | DIVMTQSPDSLAVSLGERATINCKSSKS |
| | | | VLYSNNNKNYLAWYQQKPGQPPKLLIYW |
| | | | ASTRQSGVPDRFSGSGSGTDFTLTISSL |
| | | | QAEDVAVYYCQQYLLYPLTFGGGTKVEI |
| | | | K |
| 230 | VL | CD39 | GIVMTQSPDSLAVSLGERATINCKSSQS |
| | | | VLFSSNNKNYLAWYQQKPGQPPKLLIYW |
| | | | ASTRASGVPDRFSGSGSGTDFTLTISSL |
| | | | QAEDVAVYYCQQYYLYPLTFGGGTKVEI |
| | | | K |
| 231 | VL | CD39 | EIVMTQSPATLSVSPGERATLSCRASQS |
| | | | VGSNLAWYQQKPGQAPRLLIYGASTRAT |
| | | | GIPARFSGSGSGTEFTLTISSLQSEDFA |
| | | | VYYCQQYLIWPLTFGGGTKVEIK |
| 232 | VL | CD39 | EIVMTQSPATLSVSPGERATLSCRASQS |
| | | | VGSNLAWYQQKPGQAPRLLIYGASTRAT |
| | | | GIPARFSGSGSGTEFTLTISSLQSEDFA |
| | | | VYYCQQYLLWPLTFGGGTKVEIK |
| 233 | VL | CD39 | EIVMTQSPATLSVSPGERATLSCRASQS |
| | | | VGSNLAWYQQKPGQAPRLLIYGASTRAS |
| | | | GIPARFSGSGSGTEFTLTISSLQSEDFA |
| | | | VYYCQQYLLWPLTFGGGTKVEIK |
| 234 | VL | CD39 | DIVMTQSPDSLAVSLGERATINCKSSQS |
| | | | VLFSSNNKNYLAWYQQKPGQPPKLLIYW |
| | | | ASTRESGVPDRFSGSGSGTDFTLTISSL |
| | | | QAEDVAVYYCQQFYFYPPTFGGGTKVEI |
| | | | K |
| 235 | VL | CD39 | DIVMTQSPDSLAVSLGERATINCKSSQS |
| | | | VLYSSNNKNYLAWYQQKPGQPPKLLIYW |
| | | | ASTRESGVPDRFSGSGSGTDFTLTISSL |
| | | | QAEDVAVYYCQQAYTFPPTFGGGTKVEI |
| | | | K |
| 236 | VL | CD39 | DIQMTQSPSSLSASVGDRVTITCQASQD |
| | | | ISNYLNWYQQKPGKAPKLLIYDASNLET |
| | | | GVPSRFSGSGSGTDFTFTISSLQPEDIA |
| | | | TYYCQQYYIFPPTFGGGTKVEIK |
| 237 | VL | CD39 | EIVLTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQRNFYPPTFGGGTKVEIK |
| 238 | VL | CD39 | EIVLTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQFVLWPRTFGGGTKVEIK |
| 239 | VL | CD39 | EIVLTQSPATLSLSPGERATLSCRASQS |
| | | | VSRYLAWYQQKPGQAPRLLIYDASNRAT |
| | | | GIPARFSGSGSGTDFTLTISSLEPEDFA |
| | | | VYYCQQHVNFPLTFGGGTKVEIK |
| 240 | VL | CD39 | EIVLTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQSVFWPITFGGGTKVEIK |
| 241 | VL | CD39 | EIVMTQSPATLSVSPGERATLSCRASQS |
| | | | VGSNLAWYQQKPGQAPRLLIYGASTRAT |
| | | | GIPARFSGSGSGTEFTLTISSLQSEDFA |
| | | | VYYCQQLTKWPLTFGGGTKVEIK |
| 242 | VL | CD39 | EIVLTQSPATLSLSPGERATLSCRASQS |
| | | | VSSYLAWYQQKPGQAPRLLIYDASKRAT |
| | | | GIPARFSGSGSGTDFTLTISSLEPEDFA |
| | | | VYYCQQDVLWPLTFGGGTKVEIK |
| 243 | VL | CD39 | DIQMTQSPSTLSASVGDRVTITCRASQS |
| | | | ISSWLAWYQQKPGKAPKLLIYKASSLES |
| | | | GVPSRFSGSGSGTEFTLTISSLQPDDFA |
| | | | TYYCQQYGLFPITFGGGTKVEIK |
| 244 | VL | CD39 | EIVMTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQHTVWPITFGGGTKVEIK |
| 245 | VL | CD39 | EIVLTQSPATLSLSPGERATLSCRASQS |
| | | | VSSYLAWYQQKPGQAPRLLIYDASKRAT |
| | | | GIPARFSGSGSGTDFTLTISSLEPEDFA |
| | | | VYYCQQVLNYPLTFGGGTKVEIK |
| 246 | VL | CD39 | DIQMTQSPSSLSASVGDRVTITCQASQD |
| | | | ISNYLNWYQQKPGKAPKLLIYDASNLET |
| | | | GVPSRFSGSGSGTDFTFTISSLQPEDIA |
| | | | TYYCQQSYFLPPTFGGGTKVEIK |
| 247 | VL | CD39 | EIVLTQSPGTLSLSPGERATLSCRASQS |
| | | | VSSDYLAWYQQKPGQAPRLLIYGASSRA |
| | | | TGIPDRFSGSGSGTDFTLTISRLEPEDF |
| | | | AVYYCQQAHSSPYTFGGGTKVEIK |
| 248 | VL | PD-1(181) | DVVMTQSPLSLPVTPGEPASISCRSSQS |
| | | | IVHSHGDTYLEWYLQKPGQSPQLLIYKV |
| | | | SNRFSGVPDRFSGSGSGTDFTLKISRVE |
| | | | AEDVGVYYCFQGSHIPVTFGQGTKLEIK |
| 249 | VL | PD-1 (Nivolumab) | EIVLTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQSSNWPRTFGQGTKVEIK |
| 250 | VL | PD-1 | EIVLTQSPATLSLSPGERATLSCRASKG |
| | | (Pembrolizumab) | VSTSGYSYLHWYQQKPGQAPRLLIYLAS |
| | | | YLESGVPARFSGSGSGTDFTLTISSLEP |
| | | | EDFAVYYCQHSRDLPLTFGGGTKVEIK |
| 251 | VL | PD-1 | DIQMTQSPSSLSASVGDSITITCRASLS |
| | | (Cemiplimab) | INTFLNWYQQKPGKAPNLLIYAASSLHG |
| | | | GVPSRFSGSGSGTDFTLTIRTLQPEDFA |
| | | | TYYCQQSSNTPFTFGPGTVVDFR |
| 252 | VL | PD-L1 | DIQMTQSPSSLSASVGDRVTITCRASQD |
| | | (Atezolizumab) | VSTAVAWYQQKPGKAPKLLIYSASFLYS |
| | | | GVPSRFSGSGSGTDFTLTISSLQPEDFA |
| | | | TYYCQQYLYHPATFGQGTKVEIK |
| 253 | VL | PD-L1 | QSALTQPASVSGSPGQSITISCTGTSSD |
| | | (Avelumab) | VGGYNYVSWYQQHPGKAPKLMIYDVSNR |
| | | | PSGVSNRFSGSKSGNTASLTISGLQAED |
| | | | EADYYCSSYTSSSTRVFGTGTK |
| 254 | VL | PD-L1 | EIVLTQSPGTLSLSPGERATLSCRASQR |
| | | (Durvalumab) | VSSSYLAWYQQKPGQAPRLLIYDASSRA |
| | | | TGIPDRFSGSGSGTDFTLTISRLEPEDF |
| | | | AVYYCQQYGSLPWTFGQGTKVEIK |
| 255 | HC | CD39 | QVQLVQSGAEVKKPGASVKVSCKASGYT |
| | | | FKSYEMHWVRQAPGQGLEWMGRINPSVG |
| | | | STWYAQKFQGRVTMTRDTSTSTVYMELS |
| | | | SLRSEDTAVYYCARGKREGGTEYLRKWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 256 | LC | CD39 | EIVLTQSPGTLSLSPGERATLSCRASQS |
| | | | VASSYLAWYQQKPGQAPRLLIYGASNRH |
| | | | TGIPDRFSGSGSGTDFTLTISRLEPEDF |
| | | | AVYYCQQYHNAITFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 257 | HC | CD39 | QVQLVQSGAEVKKPGASVKVSCKASGYT |
| | | | FTSYQMHWVRQAPGQGLEWMGRINPSGG |
| | | | STWYAQKFQGRVTMTRDTSTSTVYMELS |
| | | | SLRSEDTAVYYCARGKREGGTEYLRSWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 258 | LC | CD39 | EIVLTQSPGTLSLSPGERATLSCRASQS |
| | | | VSSSYLAWYQQKPGQAPRLLIYGASSRA |
| | | | TGIPDRFSGSGSGTDFTLTISRLEPEDF |
| | | | AVYYCQQYHSYITFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 259 | HC | CD39 | QVQLVQSGAEVKKPGASVKVSCKASGYI |
| | | | FKSYEMHWVRQAPGQGLEWMGRINPSVG |
| | | | STWYAQKFQGRVTMTRDTSTSTVYMELS |
| | | | SLRSEDTAVYYCARGKREGGTEYLRVWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 260 | LC | CD39 | EIVLTQSPGTLSLSPGERATLSCRASQS |
| | | | VSSSYLAWYQQKPGQAPRLLIYYASSRA |
| | | | YGIPDRFSGSGSGTDFTLTISRLEPEDF |
| | | | AVYYCQQYHNAITFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 261 | HC | CD39 | QVQLVQSGAEVKKPGASVKVSCKASGYT |
| | | | FKSYEMHWVRQAPGQGLEWMGRINPSGG |
| | | | STWYAQKFQGRVTMTRDTSTSTVYMELS |
| | | | SLRSEDTAVYYCARGKREGGTEYLRHWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 262 | LC | CD39 | EIVLTQSPGTLSLSPGERATLSCRASQS |
| | | | VSSSYLAWYQQKPGQAPRLLIYGASSRA |
| | | | TGIPDRFSGSGSGTDFTLTISRLEPEDF |
| | | | AVYYCQQYYFYITFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 263 | HC | CD39 | QVQLVQSGAEVKKPGASVKVSCKASGYT |
| | | | FKSYEMHWVRQAPGQGLEWMGRINPSGG |
| | | | STWYAQKFQGRVTMTRDTSTSTVYMELS |
| | | | SLRSEDTAVYYCARGKREGGTEYLRHWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 264 | LC | CD39 | EIVLTQSPGTLSLSPGERATLSCEASQS |
| | | | VSYSYLAWYQQKPGQAPRLLIYGASSRA |
| | | | NGIPDRFSGSGSGTDFTLTISRLEPEDF |
| | | | AVYYCQQYHSALTFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 265 | HC | CD39 | QVQLVQSGAEVKKPGSSVKVSCKASGGT |
| | | | FSSLPISWVRQAPGQGLEWMGGIGFGTA |
| | | | NYAQKFQGRVTITADESTSTAYMELSSL |
| | | | RSEDTAVYYCARGGAKYASKWGMDVWGQ |
| | | | GTTVTVSSASTKGPSVFPLAPCSRSTSE |
| | | | STAALGCLVKDYFPEPVTVSWNSGALTS |
| | | | GVHTFPAVLQSSGLYSLSSWTVPSSSL |
| | | | GTKTYTCNVDHKPSNTKVDKRVESKYGP |
| | | | PCPPCPAPEFLGGPSVFLFPPKPKDTLM |
| | | | ISRTPEVTCWVDVSQEDPEVQFNWYVD |
| | | | GVEVHNAKTKPREEQFNSTYRWSVLTV |
| | | | LHQDWLNGKEYKCKVSNKGLPSSIEKTI |
| | | | SKAKGQPREPQVYTLPPSQEEMTKNQVS |
| | | | LTCLVKGFYPSDIAVEWESNGQPENNYK |
| | | | TTPPVLDSDGSFFLYSRLTVDKSRWQEG |
| | | | NVFSCSVMHEALHNHYTQKSLSLSLGK |
| 266 | LC | CD39 | DIVMTQSPDSLAVSLGERATINCKSSQS |
| | | | VLFSSNNKNYLAWYQQKPGQPPKLLIYW |
| | | | ASSRESGVPDRFSGSGSGTDFTLTISSL |
| | | | QAEDVAVYYCQQYWTYPLTFGGGTKVEI |
| | | | KRTVAAPSVFIFPPSDEQLKSGTASWC |
| | | | LLNNFYPREAKVQWKVDNALQSGNSQES |
| | | | VTEQDSKDSTYSLSSTLTLSKADYEKHK |
| | | | VYACEVTHQGLSSPVTKSFNRGEC |
| 267 | HC | CD39 | QVQLVQSGAEVKKPGSSVKVSCKASGGT |
| | | | FPSNAISWVRQAPGQGLEWMGGIGFGTA |
| | | | NYAQKFQGRVTITADESTSTAYMELSSL |
| | | | RSEDTAVYYCARGGAKYARTYGMDVWGQ |
| | | | GTTVTVSSASTKGPSVFPLAPCSRSTSE |
| | | | STAALGCLVKDYFPEPVTVSWNSGALTS |
| | | | GVHTFPAVLQSSGLYSLSSWTVPSSSL |
| | | | GTKTYTCNVDHKPSNTKVDKRVESKYGP |
| | | | PCPPCPAPEFLGGPSVFLFPPKPKDTLM |
| | | | ISRTPEVTCWVDVSQEDPEVQFNWYVD |
| | | | GVEVHNAKTKPREEQFNSTYRWSVLTV |
| | | | LHQDWLNGKEYKCKVSNKGLPSSIEKTI |
| | | | SKAKGQPREPQVYTLPPSQEEMTKNQVS |
| | | | LTCLVKGFYPSDIAVEWESNGQPENNYK |
| | | | TTPPVLDSDGSFFLYSRLTVDKSRWQEG |
| | | | NVFSCSVMHEALHNHYTQKSLSLSLGK |
| 268 | LC | CD39 | DIVMTQSPDSLAVSLGERATINCKSSKS |
| | | | VLYSNNNKNYLAWYQQKPGQPPKLLIYW |
| | | | ASTRQSGVPDRFSGSGSGTDFTLTISSL |
| | | | QAEDVAVYYCQQYLLYPLTFGGGTKVEI |
| | | | KRTVAAPSVFIFPPSDEQLKSGTASWC |
| | | | LLNNFYPREAKVQWKVDNALQSGNSQES |
| | | | VTEQDSKDSTYSLSSTLTLSKADYEKHK |
| | | | VYACEVTHQGLSSPVTKSFNRGEC |
| 269 | HC | CD39 | QVQLVQSGAEVKKPGSSVKVSCKASGGT |
| | | | FSSLPISWVRQAPGQGLEWMGGIGFGTA |
| | | | NYAQKFQGRVTITADESTSTAYMELSSL |
| | | | RSEDTAVYYCARGGAKYAGRYGMDVWGQ |
| | | | GTTVTVSSASTKGPSVFPLAPCSRSTSE |
| | | | STAALGCLVKDYFPEPVTVSWNSGALTS |
| | | | GVHTFPAVLQSSGLYSLSSWTVPSSSL |
| | | | GTKTYTCNVDHKPSNTKVDKRVESKYGP |
| | | | PCPPCPAPEFLGGPSVFLFPPKPKDTLM |
| | | | ISRTPEVTCWVDVSQEDPEVQFNWYVD |
| | | | GVEVHNAKTKPREEQFNSTYRWSVLTV |
| | | | LHQDWLNGKEYKCKVSNKGLPSSIEKTI |
| | | | SKAKGQPREPQVYTLPPSQEEMTKNQVS |
| | | | LTCLVKGFYPSDIAVEWESNGQPENNYK |
| | | | TTPPVLDSDGSFFLYSRLTVDKSRWQEG |
| | | | NVFSCSVMHEALHNHYTQKSLSLSLGK |
| 270 | LC | CD39 | GIVMTQSPDSLAVSLGERATINCKSSQS |
| | | | VLFSSNNKNYLAWYQQKPGQPPKLLIYW |
| | | | ASTRASGVPDRFSGSGSGTDFTLTISSL |
| | | | QAEDVAVYYCQQYYLYPLTFGGGTKVEI |
| | | | KRTVAAPSVFIFPPSDEQLKSGTASVVC |
| | | | LLNNFYPREAKVQWKVDNALQSGNSQES |
| | | | VTEQDSKDSTYSLSSTLTLSKADYEKHK |
| | | | VYACEVTHQGLSSPVTKSFNRGEC |
| 271 | HC | CD39 | QVQLVQSGAEVKKPGSSVKVSCKASGGT |
| | | | FGSYGISWVRQAPGQGLEWMGSIIPEFG |
| | | | IANYAQKFQGRVTITADESTSTAYMELS |
| | | | SLRSEDTAVYYCARESGTYRDHRLDVWG |
| | | | QGTMVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 272 | LC | CD39 | EIVMTQSPATLSVSPGERATLSCRASQS |
| | | | VGSNLAWYQQKPGQAPRLLIYGASTRAT |
| | | | GIPARFSGSGSGTEFTLTISSLQSEDFA |
| | | | VYYCQQYLLWPLTFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 273 | HC | CD39 | QVQLVQSGAEVKKPGSSVKVSCKASGGT |
| | | | FSKYGISWVRQAPGQGLEWMGSIIPEFG |
| | | | IANYAQKFQGRVTITADESTSTAYMELS |
| | | | SLRSEDTAVYYCARESGGYRDHRLGVWG |
| | | | QGTMVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 274 | LC | CD39 | EIVMTQSPATLSVSPGERATLSCRASQS |
| | | | VGSNLAWYQQKPGQAPRLLIYGASTRAS |
| | | | GIPARFSGSGSGTEFTLTISSLQSEDFA |
| | | | VYYCQQYLLWPLTFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 275 | HC | CD39 | QVQLVQSGAEVKKPGSSVKVSCKASGGT |
| | | | FESYGISWVRQAPGQGLEWMGSIIPEFG |
| | | | IANYAQKFQGRVTITADESTSTTYMELS |
| | | | SLRSEDTAVYYCARESGGYRDHRLDVWG |
| | | | QGTMVTVSSASTKGPSVFPLAPCSRSTS |
| | | | ESTAALGCLVKDYFPEPVTVSWNSGALT |
| | | | SGVHTFPAVLQSSGLYSLSSWTVPSSS |
| | | | LGTKTYTCNVDHKPSNTKVDKRVESKYG |
| | | | PPCPPCPAPEFLGGPSVFLFPPKPKDTL |
| | | | MISRTPEVTCWVDVSQEDPEVQFNWYV |
| | | | DGVEVHNAKTKPREEQFNSTYRWSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 276 | LC | CD39 | EIVMTQSPATLSVSPGERATLSCRASQS |
| | | | VGSNLAWYQQKPGQAPRLLIYGASTRAT |
| | | | GIPARFSGSGSGTEFTLTISSLQSEDFA |
| | | | VYYCQQYLLWPLTFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 277 | HC | CD39 | QVQLVQSGAEVKKPGSSVKVSCKASGGT |
| | | | FSSYAISWVRQAPGQGLEWMGSIIPIFG |
| | | | TANYAQKFQGRVTITADESTSTAYMELS |
| | | | SLRSEDTAVYYCAREGRGYDSSRYYKFW |
| | | | FDPWGQGTLVTVSSASTKGPSVFPLAPC |
| | | | SRSTSESTAALGCLVKDYFPEPVTVSWN |
| | | | SGALTSGVHTFPAVLQSSGLYSLSSVVT |
| | | | VPSSSLGTKTYTCNVDHKPSNTKVDKRV |
| | | | ESKYGPPCPPCPAPEFLGGPSVFLFPPK |
| | | | PKDTLMISRTPEVTCWVDVSQEDPEVQ |
| | | | FNWYVDGVEVHNAKTKPREEQFNSTYRV |
| | | | VSVLTVLHQDWLNGKEYKCKVSNKGLPS |
| | | | SIEKTISKAKGQPREPQVYTLPPSQEEM |
| | | | TKNQVSLTCLVKGFYPSDIAVEWESNGQ |
| | | | PENNYKTTPPVLDSDGSFFLYSRLTVDK |
| | | | SRWQEGNVFSCSVMHEALHNHYTQKSLS |
| | | | LSLGK |
| 278 | LC | CD39 | EIVLTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQFVLWPRTFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 279 | HC | CD39 | QVQLVQSGAEVKEPGSSVKVSCKASGGT |
| | | | FSSYATSWVRQAPGQGLEWMGGIIPISG |
| | | | TANYAQEFQGRVTITADESTSTAYMELS |
| | | | SLRSEDTAVYYCARDGGGYRHHYFDLWG |
| | | | RGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCWVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 280 | LC | CD39 | EIVLTQSPATLSLSPGERATLSCRASQS |
| | | | VSRYLAWYQQKPGQAPRLLIYDASNRAT |
| | | | GIPARFSGSGSGTDFTLTISSLEPEDFA |
| | | | VYYCQQHVNFPLTFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 281 | HC | CD39 | QVQLVQSGAEVKKPGSSVKVPCKASGGT |
| | | | FSSYAISWVRQAPEQGLEWMGSIIPIFG |
| | | | TANYAQKFQGRVTITADESTSTAYMELS |
| | | | SLRSEDTAVYYCAGESGGYRDHKLDVWG |
| | | | QGTVVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 282 | LC | CD39 | EIVLTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQSVFWPITFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 283 | HC | CD39 | QVQLVQSGAEVKKPGSSVKVSCKASGGA |
| | | | FSSYAIGWVRQAPGQGLEWMGGIIPTFG |
| | | | TANYAQKFQGRVTITADESTSTAYMELS |
| | | | SLRSEDTAVYYCARDGGGYQHHYFDLWG |
| | | | RGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 284 | LC | CD39 | EIVMTQSPATLSVSPGERATLSCRASQS |
| | | | VGSNLAWYQQKPGQAPRLLIYGASTRAT |
| | | | GIPARFSGSGSGTEFTLTISSLQSEDFA |
| | | | VYYCQQLTKWPLTFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 285 | HC | CD39 | QVQLVQSGAEVKKPGSSVKVSCKASGGT |
| | | | FSSYAISWVRQAPGQGLEWMGSIIPIFG |
| | | | TANYAQKFQGRVTITADESTSTAYMELS |
| | | | SLRSEDTAVYYCARESGGYRDHKLDVWG |
| | | | QGTMVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRWSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 286 | LC | CD39 | EIVLTQSPATLSLSPGERATLSCRASQS |
| | | | VSSYLAWYQQKPGQAPRLLIYDASKRAT |
| | | | GIPARFSGSGSGTDFTLTISSLEPEDFA |
| | | | VYYCQQDVLWPLTFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 287 | HC | CD39 | QVQLVQSGAEVKKPGSSVKVSCKASGGT |
| | | | FSSYAISWVRQAPGQGLEWMGGIIPIFG |
| | | | TANYAQKFQGRVTITADESTSTAYMELS |
| | | | SLRSEDTAVYYCARDSGYHRHYSDYWGQ |
| | | | GTLVTVSSASTKGPSVFPLAPCSRSTSE |
| | | | STAALGCLVKDYFPEPVTVSWNSGALTS |
| | | | GVHTFPAVLQSSGLYSLSSVVTVPSSSL |
| | | | GTKTYTCNVDHKPSNTKVDKRVESKYGP |
| | | | PCPPCPAPEFLGGPSVFLFPPKPKDTLM |
| | | | ISRTPEVTCVVVDVSQEDPEVQFNWYVD |
| | | | GVEVHNAKTKPREEQFNSTYRVVSVLTV |
| | | | LHQDWLNGKEYKCKVSNKGLPSSIEKTI |
| | | | SKAKGQPREPQVYTLPPSQEEMTKNQVS |
| | | | LTCLVKGFYPSDIAVEWESNGQPENNYK |
| | | | TTPPVLDSDGSFFLYSRLTVDKSRWQEG |
| | | | NVFSCSVMHEALHNHYTQKSLSLSLGK |
| 288 | LC | CD39 | DIQMTQSPSTLSASVGDRVTITCRASQS |
| | | | ISSWLAWYQQKPGKAPKLLIYKASSLES |
| | | | GVPSRFSGSGSGTEFTLTISSLQPDDFA |
| | | | TYYCQQYGLFPITFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 289 | HC | CD39 | QVQLVQSGAEVKKPGSSVKVSCKASGGT |
| | | | FSSYAISWVRQAPGQGLEWMGGIIPIFG |
| | | | TANYAQKFQGRVTITADESTSTAYMELS |
| | | | SLRSEDTAVYYCARDPLGIRKHWFDPWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 290 | LC | CD39 | EIVMTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQHTVWPITFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 291 | HC | CD39 | QVQLVQSGAEVKKPGSSVKVSCKASGGT |
| | | | FSSYAISWVRQAPGQGLEWMGGIIPIFG |
| | | | TANYAQKFQGRVTITADESTSTAYMELS |
| | | | SLRSEDTAVYYCARDTPRWRYHYFDYWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSWTVPSSS |
| | | | LGTKTYTCNVDHKPSNTKVDKRVESKYG |
| | | | PPCPPCPAPEFLGGPSVFLFPPKPKDTL |
| | | | MISRTPEVTCWVDVSQEDPEVQFNWYV |
| | | | DGVEVHNAKTKPREEQFNSTYRWSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 292 | LC | CD39 | EIVLTQSPATLSLSPGERATLSCRASQS |
| | | | VSSYLAWYQQKPGQAPRLLIYDASKRAT |
| | | | GIPARFSGSGSGTDFTLTISSLEPEDFA |
| | | | VYYCQQVLNYPLTFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 293 | HC | CD39 | EVQLVESGGGLVKPGGSLRLSCAASGFT |
| | | | FSSYSMNWVRQAPGKGLEWVSSISSSSS |
| | | | YIYYADSVKGRFTISRDNAKNSLYLQMN |
| | | | SLRAEDTAVYYCARERRGSLALGMDVWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCWVDVSQEDPEVQFNWYV |
| | | | DGVEVHNAKTKPREEQFNSTYRWSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 294 | LC | CD39 | DIQMTQSPSSLSASVGDRVTITCQASQD |
| | | | ISNYLNWYQQKPGKAPKLLIYDASNLET |
| | | | GVPSRFSGSGSGTDFTFTISSLQPEDIA |
| | | | TYYCQQSYFLPPTFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 295 | HC | CD39 | QVQLVESGGGVVQPGRSLRLSCAASGFT |
| | | | FSSYGMNWVRQAPGKGLEWVAVIWYDGS |
| | | | NKYYADSVKGRFTISRDNSKNTLYLQMN |
| | | | SLRAEDTAVYYCARDLGGYSYGEPYYYY |
| | | | YGMDVWGQGTTVTVSSASTKGPSVFPLA |
| | | | PCSRSTSESTAALGCLVKDYFPEPVTVS |
| | | | WNSGALTSGVHTFPAVLQSSGLYSLSSV |
| | | | VTVPSSSLGTKTYTCNVDHKPSNTKVDK |
| | | | RVESKYGPPCPPCPAPEFLGGPSVFLFP |
| | | | PKPKDTLMISRTPEVTCVVVDVSQEDPE |
| | | | VQFNWYVDGVEVHNAKTKPREEQFNSTY |
| | | | RVVSVLTVLHQDWLNGKEYKCKVSNKGL |
| | | | PSSIEKTISKAKGQPREPQVYTLPPSQE |
| | | | EMTKNQVSLTCLVKGFYPSDIAVEWESN |
| | | | GQPENNYKTTPPVLDSDGSFFLYSRLTV |
| | | | DKSRWQEGNVFSCSVMHEALHNHYTQKS |
| | | | LSLSLGK |
| 296 | LC | CD39 | EIVLTQSPGTLSLSPGERATLSCRASQS |
| | | | VSSDYLAWYQQKPGQAPRLLIYGASSRA |
| | | | TGIPDRFSGSGSGTDFTLTISRLEPEDF |
| | | | AVYYCQQAHSSPYTFGGGTKVEIKRTVA |
| | | | APSVFIFPPSDEQLKSGTASVVCLLNNF |
| | | | YPREAKVQWKVDNALQSGNSQESVTEQD |
| | | | SKDSTYSLSSTLTLSKADYEKHKVYACE |
| | | | VTHQGLSSPVTKSFNRGEC |
| 297 | HC | CD39 | QVQLVQSGAEVKKPGASVKVSCKASGYT |
| | | | FKSYEMHWVRQAPGQGLEWMGRINPSVG |
| | | | STWYAQKFQGRVTMTRDTSTSTVYMELS |
| | | | SLRSEDTAVYYCARGKREGGTEYLRNWG |
| | | | QGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 298 | LC | CD39 | EIVLTQSPGTLSLSPGERATLSCRASQS |
| | | | VSSSYLAWYQQKPGQAPRLLIYGASSRA |
| | | | TGIPDRFSGSGSGTDFTLTISRLEPEDF |
| | | | AVYYCQQYHSYITFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 299 | HC | PD-1(181) | EIQLVQSGAEVKKPGSSVKVSCKASGYT |
| | | | FTHYGMNWVRQAPGQGLEWVGWVNTYTG |
| | | | EPTYADDFKGRLTFTLDTSTSTAYMELS |
| | | | SLRSEDTAVYYCTREGEGLGFGDWGQGT |
| | | | TVTVSSASTKGPSVFPLAPSSKSTSGGT |
| | | | AALGCLVKDYFPEPVTVSWNSGALTSGV |
| | | | HTFPAVLQSSGLYSLSSVVTVPSSSLGT |
| | | | QTYICNVNHKPSNTKVDKKVEPKSCDKT |
| | | | HTCPPCPAPEAAGGPSVFLFPPKPKDTL |
| | | | MISRTPEVTCVVVDVSHEDPEVKFNWYV |
| | | | DGVEVHNAKTKPREEQYNSTYRVVSVLT |
| | | | VLHQDWLNGKEYKCKVSNKALPAPIEKT |
| | | | ISKAKGQPREPQVYTLPPSREEMTKNQV |
| | | | SLTCLVKGFYPSDIAVEWESNGQPENNY |
| | | | KTTPPVLDSDGSFFLYSKLTVDKSRWQQ |
| | | | GNVFSCSVMHEALHNHYTQKSLSLSPGK |
| 300 | HC | PD-1 (181) | EIQLVQSGAEVKKPGSSVKVSCKASGYT |
| | | | FTHYGMNWVRQAPGQGLEWVGWVNTYTG |
| | | | EPTYADDFKGRLTFTLDTSTSTAYMELS |
| | | | SLRSEDTAVYYCTREGEGLGFGDWGQGT |
| | | | TVTVSSASTKGPSVFPLAPSSKSTSGGT |
| | | | AALGCLVKDYFPEPVTVSWNSGALTSGV |
| | | | HTFPAVLQSSGLYSLSSWTVPSSSLGT |
| | | | QTYICNVNHKPSNTKVDKKVEPKSCDKT |
| | | | HTCPPCPAPEAAGGPSVFLFPPKPKDTL |
| | | | MISRTPEVTCWVDVSHEDPEVKFNWYV |
| | | | DGVEVHNAKTKPREEQYNSTYRWSVLT |
| | | | VLHQDWLNGKEYKCKVSNKALPAPIEKT |
| | | | ISKAKGQPREPQVYTLPPSREEMTKNQV |
| | | | SLTCLVKGFYPSDIAVEWESNGQPENNY |
| | | | KTTPPVLDSDGSFFLYSKLTVDKSRWQQ |
| | | | GNVFSCSVMHEALHNHYTQKSLSLSPG |
| 301 | LC | PD-1(181) | DVVMTQSPLSLPVTPGEPASISCRSSQS |
| | | | IVHSHGDTYLEWYLQKPGQSPQLLIYKV |
| | | | SNRFSGVPDRFSGSGSGTDFTLKISRVE |
| | | | AEDVGVYYCFQGSHIPVTFGQGTKLEIK |
| | | | RTVAAPSVFIFPPSDEQLKSGTASWCL |
| | | | LNNFYPREAKVQWKVDNALQSGNSQESV |
| | | | TEQDSKDSTYSLSSTLTLSKADYEKHKV |
| | | | YACEVTHQGLSSPVTKSFNRGEC |
| 302 | HC | PD-1 (Nivolumab) | QVQLVESGGGWQPGRSLRLDCKASGIT |
| | | | FSNSGMHWVRQAPGKGLEWVAVIWYDGS |
| | | | KRYYADSVKGRFTISRDNSKNTLFLQMN |
| | | | SLRAEDTAVYYCATNDDYWGQGTLVTVS |
| | | | SASTKGPSVFPLAPCSRSTSESTAALGC |
| | | | LVKDYFPEPVTVSWNSGALTSGVHTFPA |
| | | | VLQSSGLYSLSSWTVPSSSLGTKTYTC |
| | | | NVDHKPSNTKVDKRVESKYGPPCPPCPA |
| | | | PEFLGGPSVFLFPPKPKDTLMISRTPEV |
| | | | TCWVDVSQEDPEVQFNWYVDGVEVHNA |
| | | | KTKPREEQFNSTYRWSVLTVLHQDWLN |
| | | | GKEYKCKVSNKGLPSSIEKTISKAKGQP |
| | | | REPQVYTLPPSQEEMTKNQVSLTCLVKG |
| | | | FYPSDIAVEWESNGQPENNYKTTPPVLD |
| | | | SDGSFFLYSRLTVDKSRWQEGNVFSCSV |
| | | | MHEALHNHYTQKSLSLSLGK |
| 303 | LC | PD-1 (Nivolumab) | EIVLTQSPATLSLSPGERATLSCRASQS |
| | | | VSSYLAWYQQKPGQAPRLLIYDASNRAT |
| | | | GIPARFSGSGSGTDFTLTISSLEPEDFA |
| | | | VYYCQQSSNWPRTFGQGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 304 | HC | PD-1 | QVQLVQSGVEVKKPGASVKVSCKASGYT |
| | | (Pembrolizumab) | FTNYYMYWVRQAPGQGLEWMGGINPSNG |
| | | | GTNFNEKFKNRVTLTTDSSTTTAYMELK |
| | | | SLQFDDTAVYYCARRDYRFDMGFDYWGQ |
| | | | GTTVTVSSASTKGPSVFPLAPCSRSTSE |
| | | | STAALGCLVKDYFPEPVTVSWNSGALTS |
| | | | GVHTFPAVLQSSGLYSLSSVVTVPSSSL |
| | | | GTKTYTCNVDHKPSNTKVDKRVESKYGP |
| | | | PCPPCPAPEFLGGPSVFLFPPKPKDTLM |
| | | | ISRTPEVTCVVVDVSQEDPEVQFNWYVD |
| | | | GVEVHNAKTKPREEQFNSTYRVVSVLTV |
| | | | LHQDWLNGKEYKCKVSNKGLPSSIEKTI |
| | | | SKAKGQPREPQVYTLPPSQEEMTKNQVS |
| | | | LTCLVKGFYPSDIAVEWESNGQPENNYK |
| | | | TTPPVLDSDGSFFLYSRLTVDKSRWQEG |
| | | | NVFSCSVMHEALHNHYTQKSLSLSLGK |
| 305 | LC | PD-1 | EIVLTQSPATLSLSPGERATLSCRASKG |
| | | (Pembrolizumab) | VSTSGYSYLHWYQQKPGQAPRLLIYLAS |
| | | | YLESGVPARFSGSGSGTDFTLTISSLEP |
| | | | EDFAVYYCQHSRDLPLTFGGGTKVEIKR |
| | | | TVAAPSVFIFPPSDEQLKSGTASVVCLL |
| | | | NNFYPREAKVQWKVDNALQSGNSQESVT |
| | | | EQDSKDSTYSLSSTLTLSKADYEKHKVY |
| | | | ACEVTHQGLSSPVTKSFNRGEC |
| 306 | HC | PD-1 | EVQLLESGGVLVQPGGSLRLSCAASGFT |
| | | (Cemiplimab) | FSNFGMTWVRQAPGKGLEWVSGISGGGR |
| | | | DTYFADSVKGRFTISRDNSKNTLYLQMN |
| | | | SLKGEDTAVYYCVKWGNIYFDYWGQGTL |
| | | | VTVSSASTKGPSVFPLAPCSRSTSESTA |
| | | | ALGCLVKDYFPEPVTVSWNSGALTSGVH |
| | | | TFPAVLQSSGLYSLSSVVTVPSSSLGTK |
| | | | TYTCNVDHKPSNTKVDKRVESKYGPPCP |
| | | | PCPAPEFLGGPSVFLFPPKPKDTLMISR |
| | | | TPEVTCVVVDVSQEDPEVQFNWYVDGVE |
| | | | VHNAKTKPREEQFNSTYRVVSVLTVLHQ |
| | | | DWLNGKEYKCKVSNKGLPSSIEKTISKA |
| | | | KGQPREPQVYTLPPSQEEMTKNQVSLTC |
| | | | LVKGFYPSDIAVEWESNGQPENNYKTTP |
| | | | PVLDSDGSFFLYSRLTVDKSRWQEGNVF |
| | | | SCSVMHEALHNHYTQKSLSLSLGK |
| 307 | LC | PD-1 | DIQMTQSPSSLSASVGDSITITCRASLS |
| | | (Cemiplimab) | INTFLNWYQQKPGKAPNLLIYAASSLHG |
| | | | GVPSRFSGSGSGTDFTLTIRTLQPEDFA |
| | | | TYYCQQSSNTPFTFGPGTVVDFRRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 308 | HC | PD-L1 | EVQLVESGGGLVQPGGSLRLSCAASGFT |
| | | (Atezolizumab) | FSDSWIHWVRQAPGKGLEWVAWISPYGG |
| | | | STYYADSVKGRFTISADTSKNTAYLQMN |
| | | | SLRAEDTAVYYCARRHWPGGFDYWGQGT |
| | | | LVTVSSASTKGPSVFPLAPSSKSTSGGT |
| | | | AALGCLVKDYFPEPVTVSWNSGALTSGV |
| | | | HTFPAVLQSSGLYSLSSWTVPSSSLGT |
| | | | QTYICNVNHKPSNTKVDKKVEPKSCDKT |
| | | | HTCPPCPAPELLGGPSVFLFPPKPKDTL |
| | | | MISRTPEVTCWVDVSHEDPEVKFNWYV |
| | | | DGVEVHNAKTKPREEQYASTYRWSVLT |
| | | | VLHQDWLNGKEYKCKVSNKALPAPIEKT |
| | | | ISKAKGQPREPQVYTLPPSREEMTKNQV |
| | | | SLTCLVKGFYPSDIAVEWESNGQPENNY |
| | | | KTTPPVLDSDGSFFLYSKLTVDKSRWQQ |
| | | | GNVFSCSVMHEALHNHYTQKSLSLSPGK |
| 309 | LC | PD-L1 | DIQMTQSPSSLSASVGDRVTITCRASQD |
| | | (Atezolizumab) | VSTAVAWYQQKPGKAPKLLIYSASFLYS |
| | | | GVPSRFSGSGSGTDFTLTISSLQPEDFA |
| | | | TYYCQQYLYHPATFGQGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 310 | HC | PD-L1 | EVQLLESGGGLVQPGGSLRLSCAASGFT |
| | | (Avelumab) | FSSYIMMWVRQAPGKGLEWVSSIYPSGG |
| | | | ITFYADTVKGRFTISRDNSKNTLYLQMN |
| | | | SLRAEDTAVYYCARIKLGTVTTVDYWGQ |
| | | | GTLVTVSSASTKGPSVFPLAPSSKSTSG |
| | | | GTAALGCLVKDYFPEPVTVSWNSGALTS |
| | | | GVHTFPAVLQSSGLYSLSSWTVPSSSL |
| | | | GTQTYICNVNHKPSNTKVDKKVEPKSCD |
| | | | KTHTCPPCPAPELLGGPSVFLFPPKPKD |
| | | | TLMISRTPEVTCWVDVSHEDPEVKFNW |
| | | | YVDGVEVHNAKTKPREEQYNSTYRWSV |
| | | | LTVLHQDWLNGKEYKCKVSNKALPAPIE |
| | | | KTISKAKGQPREPQVYTLPPSRDELTKN |
| | | | QVSLTCLVKGFYPSDIAVEWESNGQPEN |
| | | | NYKTTPPVLDSDGSFFLYSKLTVDKSRW |
| | | | QQGNVFSCSVMHEALHNHYTQKSLSLSP |
| | | | GK |
| 311 | LC | PD-L1 | QSALTQPASVSGSPGQSITISCTGTSSD |
| | | (Avelumab) | VGGYNYVSWYQQHPGKAPKLMIYDVSNR |
| | | | PSGVSNRFSGSKSGNTASLTISGLQAED |
| | | | EADYYCSSYTSSSTRVFGTGTKVTVLGQ |
| | | | PKANPTVTLFPPSSEELQANKATLVCLI |
| | | | SDFYPGAVTVAWKADGSPVKAGVETTKP |
| | | | SKQSNNKYAASSYLSLTPEQWKSHRSYS |
| | | | CQVTHEGSTVEKTVAPTECS |
| 312 | HC | PD-L1 | EVQLVESGGGLVQPGGSLRLSCAASGFT |
| | | (Durvalumab) | FSRYWMSWVRQAPGKGLEWVANIKQDGS |
| | | | EKYYVDSVKGRFTISRDNAKNSLYLQMN |
| | | | SLRAEDTAVYYCAREGGWFGELAFDYWG |
| | | | QGTLVTVSSASTKGPSVFPLAPSSKSTS |
| | | | GGTAALGCLVKDYFPEPVTVSWNSGALT |
| | | | SGVHTFPAVLQSSGLYSLSSWTVPSSS |
| | | | LGTQTYICNVNHKPSNTKVDKRVEPKSC |
| | | | DKTHTCPPCPAPEFEGGPSVFLFPPKPK |
| | | | DTLMISRTPEVTCWVDVSHEDPEVKFN |
| | | | WYVDGVEVHNAKTKPREEQYNSTYRWS |
| | | | VLTVLHQDWLNG |
| | | | KEYKCKVSNKALPASIEKTISKAKGQPR |
| | | | EPQVYTLPPSREEMTKNQVSLTCLVKGF |
| | | | YPSDIAVEWESNGQPENNYKTTPPVLDS |
| | | | DGSFFLYSKLTVDKSRWQQGNVFSCSVM |
| | | | HEALHNHYTQKSLSLSPGK |
| 313 | LC | PD-L1 | EIVLTQSPGTLSLSPGERATLSCRASQR |
| | | (Durvalumab) | VSSSYLAWYQQKPGQAPRLLIYDASSRA |
| | | | TGIPDRFSGSGSGTDFTLTISRLEPEDF |
| | | | AVYYCQQYGSLPWTFGQGTKVEIKRTVA |
| | | | APSVFIFPPSDEQLKSGTASVVCLLNNF |
| | | | YPREAKVQWKVDNALQSGNSQESVTEQD |
| | | | SKDSTYSLSSTLTLSKADYEKHKVYACE |
| | | | VTHQGLSSPVTKSFNRGEC |
| 314 | | | |
| 315 | CDR-H1 | CD39 | SYRMN |
| 316 | CDR-H1 | CD39 | DKAIS |
| 317 | CDR-H1 | CD39 | SEGIS |
| 318 | CDR-H1 | CD39 | TYAIG |
| 319 | CDR-H1 | CD39 | SWYMH |
| 320 | | | |
| 321 | CDR-H2 | CD39 | SISSSSSSIWYADSVKG |
| 322 | CDR-H2 | CD39 | SILPIFGTANYAQKFQG |
| 323 | CDR-H2 | CD39 | SILPIFGTANYAQKFQG |
| 324 | CDR-H2 | CD39 | GIIPAFGTANYAQKFQG |
| 325 | CDR-H2 | CD39 | MINPSGGSTKYAQKFQG |
| 326 | | | |
| 327 | CDR-H3 | CD39 | GPRYDSSGYRWRYGMDV |
| 328 | CDR-H3 | CD39 | EAGYYRYRYFDL |
| 329 | CDR-H3 | CD39 | EAGYYRYRYFDL |
| 330 | CDR-H3 | CD39 | DPVRRSPFDI |
| 331 | CDR-H3 | CD39 | DAPFYTWDHYYGMDV |
| 332 | | | |
| 333 | CDR-L1 | CD39 | RASQSISSYLN |
| 334 | CDR-L1 | CD39 | RASQSVSSNLA |
| 335 | CDR-L1 | CD39 | RASQSVSSNLA |
| 336 | CDR-L1 | CD39 | RASQSVSSYLA |
| 337 | CDR-L1 | CD39 | QASQDISNYLN |
| 338 | | | |
| 339 | CDR-L2 | CD39 | AASSLQS |
| 340 | CDR-L2 | CD39 | GASTRAT |
| 341 | CDR-L2 | CD39 | GASTRAT |
| 342 | CDR-L2 | CD39 | DSSNRAT |
| 343 | CDR-L2 | CD39 | DASNLAT |
| 344 | | | |
| 345 | CDR-L3 | CD39 | QQLYVDPPWT |
| 346 | CDR-L3 | CD39 | QQHALWPLT |
| 347 | CDR-L3 | CD39 | QQHALWPLT |
| 348 | CDR-L3 | CD39 | QQSFLWPRT |
| 349 | CDR-L3 | CD39 | QQLYHLPIT |
| 350 | | | |
| 351 | VH | CD39 (SRF360) | EVQLVESGGGLVKPGGSLRLSCAASGFT |
| | | | FSSYRMNWVRQAPGKGLEWVSSISSSSS |
| | | | SIWYADSVKGRFTISRDNAKNSLYLQMN |
| | | | SLRAEDTAVYYCAKGPRYDSSGYRWRYG |
| | | | MDVWGQGTTVSS |
| 352 | VL | CD39 (SRF360) | DIQMTQSPSSLSASVGDRVTITCRASQS |
| | | | ISSYLNWYQQKPGKAPKLLIYAASSLQS |
| | | | GVPSRFSGSGSGTDFTLTISSLQPEDFA |
| | | | TYYCQQLYVDPPWTFGGGTKVEIK |
| 353 | HC | CD39 (SRF360)- | EVQLVESGGGLVKPGGSLRLSCAASGFT |
| | | G4 | FSSYRMNWVRQAPGKGLEWVSSISSSSS |
| | | | SIWYADSVKGRFTISRDNAKNSLYLQMN |
| | | | SLRAEDTAVYYCAKGPRYDSSGYRWRYG |
| | | | MDVWGQGTTVSSASTKGPSVFPLAPCSR |
| | | | STSESTAALGCLVKDYFPEPVTVSWNSG |
| | | | ALTSGVHTFPAVLQSSGLYSLSSVVTVP |
| | | | SSSLGTKTYTCNVDHKPSNTKVDKRVES |
| | | | KYGPPCPPCPAPEFLGGPSVFLFPPKPK |
| | | | DTLMISRTPEVTCVVVDVSQEDPEVQFN |
| | | | WYVDGVEVHNAKTKPREEQFNSTYRWS |
| | | | VLTVLHQDWLNGKEYKCKVSNKGLPSSI |
| | | | EKTISKAKGQPREPQVYTLPPSQEEMTK |
| | | | NQVSLTCLVKGFYPSDIAVEWESNGQPE |
| | | | NNYKTTPPVLDSDGSFFLYSRLTVDKSR |
| | | | WQEGNVFSCSVMHEALHNHYTQKSLSLS |
| | | | LGK |
| 354 | LC | CD39 (SRF360) | DIQMTQSPSSLSASVGDRVTITCRASQS |
| | | | ISSYLNWYQQKPGKAPKLLIYAASSLQS |
| | | | GVPSRFSGSGSGTDFTLTISSLQPEDFA |
| | | | TYYCQQLYVDPPWTFGGGTKVEIKRTVA |
| | | | APSVFIFPPSDEQLKSGTASWCLLNNF |
| | | | YPREAKVQWKVDNALQSGNSQESVTEQD |
| | | | SKDSTYSLSSTLTLSKADYEKHKVYACE |
| | | | VTHQGLSSPVTKSFNRGEC |
| 355 | VH | CD39 (SRF365) | QVQLVQSGAEVKKPGSSVKVSCKASGGT |
| | | | FSDKAISWVRQAPGQGLEWMGSILPIFG |
| | | | TANYAQKFQGRVTITADESTSTAYMELS |
| | | | SLRSEDTAVYYCAREAGYYRYRYFDLWG |
| | | | RGTLVTVSS |
| 356 | VL | CD39 (SRF365) | EIVMTQSPATLSVSPGERATLSCRASQS |
| | | | VSSNLAWYQQKPGQAPRLLIYGASTRAT |
| | | | GIPARFSGSGSGTEFTLTISSLQSEDFA |
| | | | VYYCQQHALWPLTFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 357 | HC | CD39 (SRF365) | QVQLVQSGAEVKKPGSSVKVSCKASGGT |
| | | | FSDKAISWVRQAPGQGLEWMGSILPIFG |
| | | | TANYAQKFQGRVTITADESTSTAYMELS |
| | | | SLRSEDTAVYYCAREAGYYRYRYFDLWG |
| | | | RGTLVTVSSASTKGPSVFPLAPCSRSTS |
| | | | ESTAALGCLVKDYFPEPVTVSWNSGALT |
| | | | SGVHTFPAVLQSSGLYSLSSVVTVPSSS |
| | | | LGTKTYTCNVDHKPSNTKVDKRVESKYG |
| | | | PPCPPCPAPEFLGGPSVFLFPPKPKDTL |
| | | | MISRTPEVTCVVVDVSQEDPEVQFNWYV |
| | | | DGVEVHNAKTKPREEQFNSTYRVVSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 358 | LC | CD39 (SRF365) | EIVMTQSPATLSVSPGERATLSCRASQS |
| | | | VSSNLAWYQQKPGQAPRLLIYGASTRAT |
| | | | GIPARFSGSGSGTEFTLTISSLQSEDFA |
| | | | VYYCQQHALWPLTFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 359 | VH | CD39 (SRF367) | QVQLVQSGAEVKKPGSSVKVSCKASGGT |
| | | | FSSEGISWVRQAPGQGLEWMGSILPIFG |
| | | | TANYAQKFQGRVTITADESTSTAYMELS |
| | | | SLRSEDTAVYYCAREAGYYRYRYFDLWG |
| | | | KGTLVTVSS |
| 360 | VL | CD39 (SRF367) | EIVMTQSPATLSVSPGERATLSCRASQS |
| | | | VSSNLAWYQQKPGQAPRLLIYGASTRAT |
| | | | GIPARFSGSGSGTEFTLTISSLQSEDFA |
| | | | VYYCQQHALWPLTFGGGTKVEIK |
| 361 | HC | CD39 (SRF367) | QVQLVQSGAEVKKPGSSVKVSCKASGGT |
| | | | FSSEGISWVRQAPGQGLEWMGSILPIFG |
| | | | TANYAQKFQGRVTITADESTSTAYMELS |
| | | | SLRSEDTAVYYCAREAGYYRYRYFDLWG |
| | | | KGTLVTVSSASTKGPSVFPLAPCSRSTS |
| | | | ESTAALGCLVKDYFPEPVTVSWNSGALT |
| | | | SGVHTFPAVLQSSGLYSLSSWTVPSSS |
| | | | LGTKTYTCNVDHKPSNTKVDKRVESKYG |
| | | | PPCPPCPAPEFLGGPSVFLFPPKPKDTL |
| | | | MISRTPEVTCWVDVSQEDPEVQFNWYV |
| | | | DGVEVHNAKTKPREEQFNSTYRWSVLT |
| | | | VLHQDWLNGKEYKCKVSNKGLPSSIEKT |
| | | | ISKAKGQPREPQVYTLPPSQEEMTKNQV |
| | | | SLTCLVKGFYPSDIAVEWESNGQPENNY |
| | | | KTTPPVLDSDGSFFLYSRLTVDKSRWQE |
| | | | GNVFSCSVMHEALHNHYTQKSLSLSLGK |
| 362 | LC | CD39 (SRF367) | EIVMTQSPATLSVSPGERATLSCRASQS |
| | | | VSSNLAWYQQKPGQAPRLLIYGASTRAT |
| | | | GIPARFSGSGSGTEFTLTISSLQSEDFA |
| | | | VYYCQQHALWPLTFGGGTKVEIKRTVAA |
| | | | PSVFIFPPSDEQLKSGTASWCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 363 | VH | CD39 (SRF370) | QVQLVQSGAEVKKPGSSVKVSCKASGGT |
| | | | FSTYAIGWVRQAPGQGLEWMGGIIPAFG |
| | | | TANYAQKFQGRVTITADESTSTAYMELS |
| | | | SLRSEDTAVYYCARDPVRRSPFDIWGQG |
| | | | TMVTVSS |
| 364 | VL | CD39 (SRF370) | EIVLTQSPATLSLSPGERATLSCRASQS |
| | | | VSSYLAWYQQKPGQAPRLLIYDSSNRAT |
| | | | GIPARFSGSGSGTDFTLTISSLEPEDFA |
| | | | VYYCQQSFLWPRTFGGGTKVEIK |
| 365 | HC | CD39 (SRF370) | QVQLVQSGAEVKKPGSSVKVSCKASGGT |
| | | | FSTYAIGWVRQAPGQGLEWMGGIIPAFG |
| | | | TANYAQKFQGRVTITADESTSTAYMELS |
| | | | SLRSEDTAVYYCARDPVRRSPFDIWGQG |
| | | | TMVTVSSASTKGPSVFPLAPCSRSTSES |
| | | | TAALGCLVKDYFPEPVTVSWNSGALTSG |
| | | | VHTFPAVLQSSGLYSLSSVVTVPSSSLG |
| | | | TKTYTCNVDHKPSNTKVDKRVESKYGPP |
| | | | CPPCPAPEFLGGPSVFLFPPKPKDTLMI |
| | | | SRTPEVTCVVVDVSQEDPEVQFNWYVDG |
| | | | VEVHNAKTKPREEQFNSTYRVVSVLTVL |
| | | | HQDWLNGKEYKCKVSNKGLPSSIEKTIS |
| | | | KAKGQPREPQVYTLPPSQEEMTKNQVSL |
| | | | TCLVKGFYPSDIAVEWESNGQPENNYKT |
| | | | TPPVLDSDGSFFLYSRLTVDKSRWQEGN |
| | | | VFSCSVMHEALHNHYTQKSLSLSLGK |
| 366 | LC | CD39 (SRF370) | EIVLTQSPATLSLSPGERATLSCRASQS |
| | | | VSSYLAWYQQKPGQAPRLLIYDSSNRAT |
| | | | GIPARFSGSGSGTDFTLTISSLEPEDFA |
| | | | VYYCQQSFLWPRTFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| 367 | VH | CD39 (SRF399) | QVQLVQSGAEVKKPGASVKVSCKASGYT |
| | | | FSSWYMHWVRQAPGQGLEWMGMINPSGG |
| | | | STKYAQKFQGRVTMTRDTSTSTVYMELS |
| | | | SLRSEDTAVYYCARDAPFYTWDHYYGMD |
| | | | VWGQGTTVTVSS |
| 368 | VL | CD39 (SRF399) | DIQMTQSPSSLSASVGDRVTITCQASQD |
| | | | ISNYLNWYQQKPGKAPKLLIYDASNLAT |
| | | | GVPSRFSGSGSGTDFTFTISSLQPEDIA |
| | | | TYYCQQLYHLPITFGGGTKVEIK |
| 369 | HC | CD39 (SRF399) | QVQLVQSGAEVKKPGASVKVSCKASGYT |
| | | | FSSWYMHWVRQAPGQGLEWMGMINPSGG |
| | | | STKYAQKFQGRVTMTRDTSTSTVYMELS |
| | | | SLRSEDTAVYYCARDAPFYTWDHYYGMD |
| | | | VWGQGTTVTVSSASTKGPSVFPLAPCSR |
| | | | STSESTAALGCLVKDYFPEPVTVSWNSG |
| | | | ALTSGVHTFPAVLQSSGLYSLSSVVTVP |
| | | | SSSLGTKTYTCNVDHKPSNTKVDKRVES |
| | | | KYGPPCPPCPAPEFLGGPSVFLFPPKPK |
| | | | DTLMISRTPEVTCVVVDVSQEDPEVQFN |
| | | | WYVDGVEVHNAKTKPREEQFNSTYRVVS |
| | | | VLTVLHQDWLNGKEYKCKVSNKGLPSSI |
| | | | EKTISKAKGQPREPQVYTLPPSQEEMTK |
| | | | NQVSLTCLVKGFYPSDIAVEWESNGQPE |
| | | | NNYKTTPPVLDSDGSFFLYSRLTVDKSR |
| | | | WQEGNVFSCSVMHEALHNHYTQKSLSLS |
| | | | LGK |
| 370 | LC | CD39 (SRF399) | DIQMTQSPSSLSASVGDRVTITCQASQD |
| | | | ISNYLNWYQQKPGKAPKLLIYDASNLAT |
| | | | GVPSRFSGSGSGTDFTFTISSLQPEDIA |
| | | | TYYCQQLYHLPITFGGGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1. A method for treatment of a subject suffering from cancer, comprising administering to the subject a therapeutically effective amount of an antibody which binds to CD39 and a therapeutically effective amount of an antibody which binds to PD-1 and/or PD-L1.

2. The method according to claim 1, wherein the antibody that binds to CD39 comprises or consists of a heavy chain variable region (VH) and a light chain variable region (VL), with VH and/or VL comprising:
a) a VHCDR1 having the sequence set forth in any one of SEQ ID NOs: 1-21 or SEQ ID NOs: 315-319,
b) a VHCDR2 having the sequence set forth in any one of SEQ ID NOs: 32-50 or SEQ ID NOs: 321-325,
c) a VHCDR3 having the sequence set forth in any one of SEQ ID NOs: 58-85 or SEQ ID NOs: 327-331,
d) a VLCDR1 having the sequence set forth in any one of SEQ ID NOs: 93-107 or SEQ ID NOs: 333-337,
e) a VLCDR2 having the sequence set forth in any one of SEQ ID NOs: 115-130 or SEQ ID NOs: 339-343, and
f) a VLCDR3 having the sequence set forth in any one of SEQ ID NOs: 138-163 or SEQ ID NOs: 345-349.

3. The method according to claim 1, wherein the antibody that binds to CD39 comprises or consists of a heavy chain variable region (VH) and a light chain variable region (VL), with VH comprising, consisting of, or consisting essentially of a VH having the sequence set forth in SEQ ID NOs: 171-210, SEQ ID NO: 351, SEQ ID NO: 355, SEQ ID NO: 359, SEQ ID NO: 363, or SEQ ID NO: 367 and with VL comprising, consisting of, or consisting essentially of a VL having the sequence set forth in SEQ ID NO: 218-247, SEQ ID NO: 352, SEQ ID NO: 356, SEQ ID NO: 360, SEQ ID NO: 364, or SEQ ID NO: 368.

4. The method according to claim 1, wherein the antibody that binds to PD-1 comprises or consists of a heavy chain variable region (VH) and a light chain variable region (VL), VH and/or VL comprising:
a) a VHCDR1 having the sequence set forth in SEQ ID NO: 25,
b) a VHCDR2 having the sequence set forth in SEQ ID NO: 51,
c) a VHCDR3 having the sequence set forth in SEQ ID NO: 86,
d) a VLCDR1 having the sequence set forth in SEQ ID NO: 108,
e) a VLCDR2 having the sequence set forth in SEQ ID NO: 131, and
f) a VLCDR3 having the sequence set forth in SEQ ID NO: 164.

5. The method according to claim 4, wherein the antibody that binds to PD-1 comprises or consists of a heavy chain variable region (VH) and a light chain variable region (VL), with VH comprising, consisting of, or consisting essentially of a VH having the sequence set forth in SEQ ID NO: 211 and with VL comprising, consisting of, or consisting essentially of a VL having the sequence set forth in SEQ ID NO: 248.

6. The method according to claim 1, wherein the antibody that binds to PD-1 comprises or consists of a heavy chain variable region (VH) and a light chain variable region (VL), VH and/or VL comprising:
a) a VHCDR1 having the sequence set forth in SEQ ID NO: 26,
b) a VHCDR2 having the sequence set forth in SEQ ID NO: 52,
c) a VHCDR3 having the sequence set forth in SEQ ID NO: 87,
d) a VLCDR1 having the sequence set forth in SEQ ID NO: 109,
e) a VLCDR2 having the sequence set forth in SEQ ID NO: 132, and
f) a VLCDR3 having the sequence set forth in SEQ ID NO: 165.

7. The method according to claim 6, wherein the antibody that binds to PD-1 comprises or consists of a heavy chain variable region (VH) and a light chain variable region (VL), with VH comprising, consisting of, or consisting essentially of a VH having the sequence set forth in SEQ ID NO: 212 and with VL comprising, consisting of, or consisting essentially of a VL having the sequence set forth in SEQ ID NO: 249.

8. The method according to claim 1, wherein the antibody that binds to PD-1 comprises or consists of a heavy chain variable region (VH) and a light chain variable region (VL), VH and/or VL comprising:
a) a VHCDR1 having the sequence set forth in SEQ ID NO: 27,
b) a VHCDR2 having the sequence set forth in SEQ ID NO: 53,
c) a VHCDR3 having the sequence set forth in SEQ ID NO: 88,
d) a VLCDR1 having the sequence set forth in SEQ ID NO: 110,
e) a VLCDR2 having the sequence set forth in SEQ ID NO: 133, and
f) a VLCDR3 having the sequence set forth in SEQ ID NO: 166.

9. The method according to claim 8, wherein the antibody that binds to PD-1 comprises or consists of a heavy chain variable region (VH) and a light chain variable region (VL), with VH comprising, consisting of, or consisting essentially of a VH having the sequence set forth in SEQ ID NO: 213 and with VL comprising, consisting of, or consisting essentially of a VL having the sequence set forth in SEQ ID NO: 250.

10. The method according to claim 1, wherein the antibody that binds to PD-1 comprises or consists of a heavy chain variable region (VH) and a light chain variable region (VL), VH and/or VL comprising:
a) a VHCDR1 having the sequence set forth in SEQ ID NO: 28,
b) a VHCDR2 having the sequence set forth in SEQ ID NO: 54,
c) a VHCDR3 having the sequence set forth in SEQ ID NO: 89,
d) a VLCDR1 having the sequence set forth in SEQ ID NO: 111,
e) a VLCDR2 having the sequence set forth in SEQ ID NO: 134, and
f) a VLCDR3 having the sequence set forth in SEQ ID NO: 167.

11. The method according to claim 10, wherein the antibody that binds to PD-1 comprises or consists of a heavy chain variable region (VH) and a light chain variable region (VL), with VH comprising, consisting of, or consisting essentially of a VH having the sequence set forth in SEQ ID NO: 214 and with VL comprising, consisting of, or consisting essentially of a VL having the sequence set forth in SEQ ID NO: 251.

12. The method according to claim 1, wherein the antibody that binds to PD-L1 comprises or consists of a heavy chain variable region (VH) and a light chain variable region (VL), VH and/or VL comprising:
a) a VHCDR1 having the sequence set forth in SEQ ID NO: 29,
b) a VHCDR2 having the sequence set forth in SEQ ID NO: 55,
c) a VHCDR3 having the sequence set forth in SEQ ID NO: 90,
d) a VLCDR1 having the sequence set forth in SEQ ID NO: 112,
e) a VLCDR2 having the sequence set forth in SEQ ID NO: 135, and
f) a VLCDR3 having the sequence set forth in SEQ ID NO: 168.

13. The method according to claim 12, wherein the antibody that binds to PD-L1 comprises or consists of a heavy chain variable region (VH) and a light chain variable region (VL), with VH comprising, consisting of, or consisting essentially of a VH having the sequence set forth in SEQ ID NO: 215 and with VL comprising, consisting of, or consisting essentially of a VL having the sequence set forth in SEQ ID NO: 252.

14. The method according to claim 1, wherein the antibody that binds to PD-L1 comprises or consists of a heavy chain variable region (VH) and a light chain variable region (VL), VH and/or VL comprising:
a) a VHCDR1 having the sequence set forth in SEQ ID NO: 30,
b) a VHCDR2 having the sequence set forth in SEQ ID NO: 56,
c) a VHCDR3 having the sequence set forth in SEQ ID NO: 91,
d) a VLCDR1 having the sequence set forth in SEQ ID NO: 113,
e) a VLCDR2 having the sequence set forth in SEQ ID NO: 136, and
f) a VLCDR3 having the sequence set forth in SEQ ID NO: 169.

15. The method according to claim 14, wherein the antibody that binds to PD-L1 comprises or consists of a heavy chain variable region (VH) and a light chain variable region (VL), with VH comprising, consisting of, or consisting essentially of a VH having the sequence set forth in SEQ ID NO: 216 and with VL comprising, consisting of, or consisting essentially of a VL having the sequence set forth in SEQ ID NO: 253.

16. The method according to claim 1, wherein the antibody that binds to PD-L1 comprises or consists of a heavy chain variable region (VH) and a light chain variable region (VL), VH and/or VL comprising:
a) a VHCDR1 having the sequence set forth in SEQ ID NO: 31,
b) a VHCDR2 having the sequence set forth in SEQ ID NO: 57,
c) a VHCDR3 having the sequence set forth in SEQ ID NO: 92,
d) a VLCDR1 having the sequence set forth in SEQ ID NO: 114,
e) a VLCDR2 having the sequence set forth in SEQ ID NO: 137, and
f) a VLCDR3 having the sequence set forth in SEQ ID NO: 170.

17. The method according to claim 16, wherein the antibody that binds to PD-L1 comprises or consists of a heavy chain variable region (VH) and a light chain variable region (VL), with VH comprising, consisting of, or consisting essentially of a VH having the sequence set forth in SEQ ID NO: 217 and with VL comprising, consisting of, or consisting essentially of a VL having the sequence set forth in SEQ ID NO: 254.

18. The method according to claim 1, wherein the cancer is a solid cancer selected from the group consisting of metastatic non-small cell lung cancer (NSCLC), metastatic head and neck squamous cell carcinoma (HNSCC), melanoma, renal cell carcinoma, metastatic cutaneous squamous cell carcinoma, Hodgkin's lymphoma, and unresectable or metastatic solid tumor with DNA mismatch repair deficiencies or a microsatellite instability-high state.

19. (canceled)

20. The method according to claim 18, the subject is a human subject who is recurrent or progressive after platinum therapy.

21. (canceled)

22. The method according to claim 1, wherein the method enhances pro-inflammatory cytokine secretion, wherein the cytokines are one or more of the cytokines selected from IL-2, IFN-γ, or TNF-α; and/or the method enhances T-cell proliferation and/or cytotoxicity in comparison to a pharmaceutical composition that comprises the immune checkpoint modulator and does not comprise the anti-CD39 antibody, wherein the T cells comprise or consist of CD4⁺ cells and/or CD8⁺ cells.

23.-27. (canceled)


SEQUENCE LISTING (370 sequences)

SEQ ID NO: 1 - protein, 5 aa, synthetic construct
  REGION 1..5: note = Synthetic: CDR-H1, CD39
SYYMH

SEQ ID NO: 2 - protein, 5 aa, synthetic construct
  REGION 1..5: note = Synthetic: CDR-H1, CD39
SYEMH

SEQ ID NO: 3 - protein, 5 aa, synthetic construct
  REGION 1..5: note = Synthetic: CDR-H1, CD39
SYQMH

SEQ ID NO: 4 - protein, 5 aa, synthetic construct
  REGION 1..5: note = Synthetic: CDR-H1, CD39
SYYMY

SEQ ID NO: 5 - protein, 5 aa, synthetic construct
  REGION 1..5: note = Synthetic: CDR-H1, CD39
SYFMH

SEQ ID NO: 6 - protein, 5 aa, synthetic construct
  REGION 1..5: note = Synthetic: CDR-H1, CD39
SLAIS

SEQ ID NO: 7 - protein, 5 aa, synthetic construct
  REGION 1..5: note = Synthetic: CDR-H1, CD39
KLAIS

SEQ ID NO: 8 - protein, 5 aa, synthetic construct
  REGION 1..5: note = Synthetic: CDR-H1, CD39
HTAIS

SEQ ID NO: 9 - protein, 5 aa, synthetic construct
  REGION 1..5: note = Synthetic: CDR-H1, CD39
SLPIS

SEQ ID NO: 10 - protein, 5 aa, synthetic construct
  REGION 1..5: note = Synthetic: CDR-H1, CD39
LLAIS

SEQ ID NO: 11 - protein, 5 aa, synthetic construct
  REGION 1..5: note = Synthetic: CDR-H1, CD39
SNAIS

SEQ ID NO: 12 - protein, 5 aa, synthetic construct
  REGION 1..5: note = Synthetic: CDR-H1, CD39
AMAIS

SEQ ID NO: 13 - protein, 5 aa, synthetic construct
  REGION 1..5: note = Synthetic: CDR-H1, CD39
WLAIS

SEQ ID NO: 14 - protein, 5 aa, synthetic construct
  REGION 1..5: note = Synthetic: CDR-H1, CD39
SYAIS

SEQ ID NO: 15 - protein, 5 aa, synthetic construct
  REGION 1..5: note = Synthetic: CDR-H1, CD39
SYGIS

SEQ ID NO: 16 - protein, 5 aa, synthetic construct
  REGION 1..5: note = Synthetic: CDR-H1, CD39
KYGIS

SEQ ID NO: 17 - protein, 5 aa, synthetic construct
  REGION 1..5: note = Synthetic: CDR-H1, CD39
NYAIS

SEQ ID NO: 18 - protein, 5 aa, synthetic construct
  REGION 1..5: note = Synthetic: CDR-H1, CD39
SYATS

SEQ ID NO: 19 - protein, 5 aa, synthetic construct
  REGION 1..5: note = Synthetic: CDR-H1, CD39
SYAIG

SEQ ID NO: 20 - protein, 5 aa, synthetic construct
  REGION 1..5: note = Synthetic: CDR-H1, CD39
SYSMN

SEQ ID NO: 21 - protein, 5 aa, synthetic construct
  REGION 1..5: note = Synthetic: CDR-H1, CD39
SYGMN

SEQ ID NO: 22-24 - skipped (3 entries)

SEQ ID NO: 25 - protein, 5 aa, synthetic construct
  REGION 1..5: note = Synthetic: CDR-H1, PD-1 (181)
HYGMN

SEQ ID NO: 26 - protein, 5 aa, synthetic construct
  REGION 1..5: note = Synthetic: CDR-H1, PD-1 (Nivolumab)
NSGMH

SEQ ID NO: 27 - protein, 5 aa, synthetic construct
  REGION 1..5: note = Synthetic: CDR-H1, PD-1 (Pembrolizumab)
NYYMY

SEQ ID NO: 28 - protein, 5 aa, synthetic construct
  REGION 1..5: note = Synthetic: CDR-H1, PD-1 (Cemiplimab)
NFGMT

SEQ ID NO: 29 - protein, 5 aa, synthetic construct
  REGION 1..5: note = Synthetic: CDR-H1, PD-L1 (Atezolizumab)
DSWIH

SEQ ID NO: 30 - protein, 5 aa, synthetic construct
  REGION 1..5: note = Synthetic: CDR-H1, PD-L1 (Avelumab)
SYIMM

SEQ ID NO: 31 - protein, 5 aa, synthetic construct
  REGION 1..5: note = Synthetic: CDR-H1, PD-L1 (Durvalumab)
RYWMS

SEQ ID NO: 32 - protein, 17 aa, synthetic construct
  REGION 1..17: note = Synthetic: CDR-H2, CD39
VINPSGGSTSYAQKFQG

SEQ ID NO: 33 - protein, 17 aa, synthetic construct
  REGION 1..17: note = Synthetic: CDR-H2, CD39
RINPSVGSTWYAQKFQG

SEQ ID NO: 34 - protein, 17 aa, synthetic construct
  REGION 1..17: note = Synthetic: CDR-H2, CD39
RINPSGGSTWYAQKFQG

SEQ ID NO: 35 - protein, 17 aa, synthetic construct
  REGION 1..17: note = Synthetic: CDR-H2, CD39
KINPSGGSTWYAQKFQG

SEQ ID NO: 36 - protein, 17 aa, synthetic construct
  REGION 1..17: note = Synthetic: CDR-H2, CD39
VINPLGGGTSYAQKFQG

SEQ ID NO: 37 - protein, 17 aa, synthetic construct
  REGION 1..17: note = Synthetic: CDR-H2, CD39
SINPRGGSTSYAQKFQG

SEQ ID NO: 38 - protein, 17 aa, synthetic construct
  REGION 1..17: note = Synthetic: CDR-H2, CD39
GIIPIFGTANYAQKFQG

SEQ ID NO: 39 - protein, 15 aa, synthetic construct
  REGION 1..15: note = Synthetic: CDR-H2, CD39
GIGFGTANYAQKFQG

SEQ ID NO: 40 - protein, 17 aa, synthetic construct
  REGION 1..17: note = Synthetic: CDR-H2, CD39
GILPIGGTANYAQKFQG

SEQ ID NO: 41 - protein, 17 aa, synthetic construct
  REGION 1..17: note = Synthetic: CDR-H2, CD39
GILPIAGTANYAQKFQG

SEQ ID NO: 42 - protein, 17 aa, synthetic construct
  REGION 1..17: note = Synthetic: CDR-H2, CD39
GILPIFGEANYAQKFQG

SEQ ID NO: 43 - protein, 17 aa, synthetic construct
  REGION 1..17: note = Synthetic: CDR-H2, CD39
GIIPRGGTANYAQKFQG

SEQ ID NO: 44 - protein, 17 aa, synthetic construct
  REGION 1..17: note = Synthetic: CDR-H2, CD39
SIIPIFGTANYAQKFRG

SEQ ID NO: 45 - protein, 17 aa, synthetic construct
  REGION 1..17: note = Synthetic: CDR-H2, CD39
SIIPEFGIANYAQKFQG

SEQ ID NO: 46 - protein, 17 aa, synthetic construct
  REGION 1..17: note = Synthetic: CDR-H2, CD39
SIIPIFGTANYAQKFQG

SEQ ID NO: 47 - protein, 17 aa, synthetic construct
  REGION 1..17: note = Synthetic: CDR-H2, CD39
GIIPISGTANYAQEFQG

SEQ ID NO: 48 - protein, 17 aa, synthetic construct
  REGION 1..17: note = Synthetic: CDR-H2, CD39
GIIPTFGTANYAQKFQG

SEQ ID NO: 49 - protein, 17 aa, synthetic construct
  REGION 1..17: note = Synthetic: CDR-H2, CD39
SISSSSSYIYYADSVKG

SEQ ID NO: 50 - protein, 17 aa, synthetic construct
  REGION 1..17: note = Synthetic: CDR-H2, CD39
VIWYDGSNKYYADSVKG

SEQ ID NO: 51 - protein, 17 aa, synthetic construct
  REGION 1..17: note = Synthetic: CDR-H2, PD-1 (181)
WVNTYTGEPTYADDFKG

SEQ ID NO: 52 - protein, 17 aa, synthetic construct
  REGION 1..17: note = Synthetic: CDR-H2, PD-1 (Nivolumab)
VIWYDGSKRYYADSVKG

SEQ ID NO: 53 - protein, 17 aa, synthetic construct
  REGION 1..17: note = Synthetic: CDR-H2, PD-1 (Pembrolizumab)
GINPSNGGTNFNEKFKN

SEQ ID NO: 54 - protein, 17 aa, synthetic construct
  REGION 1..17: note = Synthetic: CDR-H2, PD-1 (Cemiplimab)
GISGGGRDTYFADSVKG

SEQ ID NO: 55 - protein, 17 aa, synthetic construct
  REGION 1..17: note = Synthetic: CDR-H2, PD-L1 (Atezolizumab)
WISPYGGSTYYADSVKG

SEQ ID NO: 56 - protein, 17 aa, synthetic construct
  REGION 1..17: note = Synthetic: CDR-H2, PD-L1 (Avelumab)
SIYPSGGITFYADTVKG

SEQ ID NO: 57 - protein, 17 aa, synthetic construct
  REGION 1..17: note = Synthetic: CDR-H2, PD-L1 (Durvalumab)
NIKQDGSEKYYVDSVKG

SEQ ID NO: 58 - protein, 12 aa, synthetic construct
  REGION 1..12: note = Synthetic: CDR-H3, CD39
GKREGGTEYLRH

SEQ ID NO: 59 - protein, 12 aa, synthetic construct
  REGION 1..12: note = Synthetic: CDR-H3, CD39
GKREGGTEYLRK

SEQ ID NO: 60 - protein, 12 aa, synthetic construct
  REGION 1..12: note = Synthetic: CDR-H3, CD39
GKREGGTEYLRS

SEQ ID NO: 61 - protein, 12 aa, synthetic construct
  REGION 1..12: note = Synthetic: CDR-H3, CD39
GKREGGTEYLRN

SEQ ID NO: 62 - protein, 12 aa, synthetic construct
  REGION 1..12: note = Synthetic: CDR-H3, CD39
GKREGGTEYLRV

SEQ ID NO: 63 - protein, 13 aa, synthetic construct
  REGION 1..13: note = Synthetic: CDR-H3, CD39
GGAKYASTYGMDV

SEQ ID NO: 64 - protein, 13 aa, synthetic construct
  REGION 1..13: note = Synthetic: CDR-H3, CD39
GGAKYASTHGMDV

SEQ ID NO: 65 - protein, 13 aa, synthetic construct
  REGION 1..13: note = Synthetic: CDR-H3, CD39
GGAKYASQLGMDV

SEQ ID NO: 66 - protein, 13 aa, synthetic construct
  REGION 1..13: note = Synthetic: CDR-H3, CD39
GGAKYASKWGMDV

SEQ ID NO: 67 - protein, 13 aa, synthetic construct
  REGION 1..13: note = Synthetic: CDR-H3, CD39
GGAKYAVGYGMDV

SEQ ID NO: 68 - protein, 13 aa, synthetic construct
  REGION 1..13: note = Synthetic: CDR-H3, CD39
GGAKYAGRYGMDV

SEQ ID NO: 69 - protein, 13 aa, synthetic construct
  REGION 1..13: note = Synthetic: CDR-H3, CD39
GGAKYARTYGMDV

SEQ ID NO: 70 - protein, 12 aa, synthetic construct
  REGION 1..12: note = Synthetic: CDR-H3, CD39
ESGGYRDHRLDV

SEQ ID NO: 71 - protein, 12 aa, synthetic construct
  REGION 1..12: note = Synthetic: CDR-H3, CD39
ESGTYRDHRLDV

SEQ ID NO: 72 - protein, 12 aa, synthetic construct
  REGION 1..12: note = Synthetic: CDR-H3, CD39
ESGGYRDHRLGV

SEQ ID NO: 73 - protein, 15 aa, synthetic construct
  REGION 1..15: note = Synthetic: CDR-H3, CD39
DFTDYSSGYSSGWTY

SEQ ID NO: 74 - protein, 14 aa, synthetic construct
  REGION 1..14: note = Synthetic: CDR-H3, CD39
DTLYSSGAYYGYNV

SEQ ID NO: 75 - protein, 15 aa, synthetic construct
  REGION 1..15: note = Synthetic: CDR-H3, CD39
AKRGYDSYGGVYFDY

SEQ ID NO: 76 - protein, 18 aa, synthetic construct
  REGION 1..18: note = Synthetic: CDR-H3, CD39
GPTVTATTSIGTHNWFDP

SEQ ID NO: 77 - protein, 28 aa, synthetic construct
  REGION 1..28: note = Synthetic: CDR-H3, CD39
EGRGYDSSRYYKFWFDPWGQGTLVTVSS

SEQ ID NO: 78 - protein, 12 aa, synthetic construct
  REGION 1..12: note = Synthetic: CDR-H3, CD39
DGGGYRHHYFDL

SEQ ID NO: 79 - protein, 12 aa, synthetic construct
  REGION 1..12: note = Synthetic: CDR-H3, CD39
ESGGYRDHKLDV

SEQ ID NO: 80 - protein, 12 aa, synthetic construct
  REGION 1..12: note = Synthetic: CDR-H3, CD39
DGGGYQHHYFDL

SEQ ID NO: 81 - protein, 11 aa, synthetic construct
  REGION 1..11: note = Synthetic: CDR-H3, CD39
DSGYHRHYSDY

SEQ ID NO: 82 - protein, 12 aa, synthetic construct
  REGION 1..12: note = Synthetic: CDR-H3, CD39
DPLGIRKHWFDP

SEQ ID NO: 83 - protein, 12 aa, synthetic construct
  REGION 1..12: note = Synthetic: CDR-H3, CD39
DTPRWRYHYFDY

SEQ ID NO: 84 - protein, 12 aa, synthetic construct
  REGION 1..12: note = Synthetic: CDR-H3, CD39
ERRGSLALGMDV

SEQ ID NO: 85 - protein, 19 aa, synthetic construct
  REGION 1..19: note = Synthetic: CDR-H3, CD39
DLGGYSYGEPYYYYYGMDV

SEQ ID NO: 86 - protein, 9 aa, synthetic construct
  REGION 1..9: note = Synthetic: CDR-H3, PD-1 (181)
EGEGLGFGD

SEQ ID NO: 87 - protein, 4 aa, synthetic construct
  REGION 1..4: note = Synthetic: CDR-H3, PD-1 (Nivolumab)
NDDY

SEQ ID NO: 88 - protein, 11 aa, synthetic construct
  REGION 1..11: note = Synthetic: CDR-H3, PD-1 (Pembrolizumab)
RDYRFDMGFDY

SEQ ID NO: 89 - protein, 8 aa, synthetic construct
  REGION 1..8: note = Synthetic: CDR-H3, PD-1 (Cemiplimab)
WGNIYFDY

SEQ ID NO: 90 - protein, 9 aa, synthetic construct
  REGION 1..9: note = Synthetic: CDR-H3, PD-L1 (Atezolizumab)
RHWPGGFDY

SEQ ID NO: 91 - protein, 11 aa, synthetic construct
  REGION 1..11: note = Synthetic: CDR-H3, PD-L1 (Avelumab)
IKLGTVTTVDY

SEQ ID NO: 92 - protein, 12 aa, synthetic construct
  REGION 1..12: note = Synthetic: CDR-H3, PD-L1 (Durvalumab)
EGGWFGELAFDY

SEQ ID NO: 93 - protein, 12 aa, synthetic construct
  REGION 1..12: note = Synthetic: CDR-L1, CD39
RASQSVSSSYLA

SEQ ID NO: 94 - protein, 12 aa, synthetic construct
  REGION 1..12: note = Synthetic: CDR-L1, CD39
RASQSVASSYLA

SEQ ID NO: 95 - protein, 12 aa, synthetic construct
  REGION 1..12: note = Synthetic: CDR-L1, CD39
EASQSVSYSYLA

SEQ ID NO: 96 - protein, 12 aa, synthetic construct
  REGION 1..12: note = Synthetic: CDR-L1, CD39
KASESVSSSYLA

SEQ ID NO: 97 - protein, 12 aa, synthetic construct
  REGION 1..12: note = Synthetic: CDR-L1, CD39
RASQYVSSSYLA

SEQ ID NO: 98 - protein, 17 aa, synthetic construct
  REGION 1..17: note = Synthetic: CDR-L1, CD39
KSSQSVLFSSNNKNYLA

SEQ ID NO: 99 - protein, 17 aa, synthetic construct
  REGION 1..17: note = Synthetic: CDR-L1, CD39
KSSRSVLFSSNNKNYLA

SEQ ID NO: 100 - protein, 17 aa, synthetic construct
  REGION 1..17: note = Synthetic: CDR-L1, CD39
KSSKSVLYSNNNKNYLA

SEQ ID NO: 101 - protein, 11 aa, synthetic construct
  REGION 1..11: note = Synthetic: CDR-L1, CD39
RASQSVGSNLA

SEQ ID NO: 102 - protein, 17 aa, synthetic construct
  REGION 1..17: note = Synthetic: CDR-L1, CD39
KSSQSVLYSSNNKNYLA

SEQ ID NO: 103 - protein, 11 aa, synthetic construct
  REGION 1..11: note = Synthetic: CDR-L1, CD39
QASQDISNYLN

SEQ ID NO: 104 - protein, 11 aa, synthetic construct
  REGION 1..11: note = Synthetic: CDR-L1, CD39
RASQSVSSYLA

SEQ ID NO: 105 - protein, 11 aa, synthetic construct
  REGION 1..11: note = Synthetic: CDR-L1, CD39
RASQSVSRYLA

SEQ ID NO: 106 - protein, 11 aa, synthetic construct
  REGION 1..11: note = Synthetic: CDR-L1, CD39
RASQSISSWLA

SEQ ID NO: 107 - protein, 12 aa, synthetic construct
  REGION 1..12: note = Synthetic: CDR-L1, CD39
RASQSVSSDYLA

SEQ ID NO: 108 - protein, 16 aa, synthetic construct
  REGION 1..16: note = Synthetic: CDR-L1, PD-1 (181)
RSSQSIVHSHGDTYLE

SEQ ID NO: 109 - protein, 11 aa, synthetic construct
  REGION 1..11: note = Synthetic: CDR-L1, PD-1 (Nivolumab)
RASQSVSSYLA

SEQ ID NO: 110 - protein, 15 aa, synthetic construct
  REGION 1..15: note = Synthetic: CDR-L1, PD-1 (Pembrolizumab)
RASKGVSTSGYSYLH

SEQ ID NO: 111 - protein, 11 aa, synthetic construct
  REGION 1..11: note = Synthetic: CDR-L1, PD-1 (Cemiplimab)
RASLSINTFLN

SEQ ID NO: 112 - protein, 11 aa, synthetic construct
  REGION 1..11: note = Synthetic: CDR-L1, PD-L1 (Atezolizumab)
RASQDVSTAVA

SEQ ID NO: 113 - protein, 14 aa, synthetic construct
  REGION 1..14: note = Synthetic: CDR-L1, PD-L1 (Avelumab)
TGTSSDVGGYNYVS

SEQ ID NO: 114 - protein, 12 aa, synthetic construct
  REGION 1..12: note = Synthetic: CDR-L1, PD-L1 (Durvalumab)
RASQRVSSSYLA

SEQ ID NO: 115 - protein, 7 aa, synthetic construct
  REGION 1..7: note = Synthetic: CDR-L2, CD39
GASSRAT

SEQ ID NO: 116 - protein, 7 aa, synthetic construct
  REGION 1..7: note = Synthetic: CDR-L2, CD39
GASNRHT

SEQ ID NO: 117 - protein, 7 aa, synthetic construct
  REGION 1..7: note = Synthetic: CDR-L2, CD39
YASSRAY

SEQ ID NO: 118 - protein, 7 aa, synthetic construct
  REGION 1..7: note = Synthetic: CDR-L2, CD39
GASSRAN

SEQ ID NO: 119 - protein, 7 aa, synthetic construct
  REGION 1..7: note = Synthetic: CDR-L2, CD39
YASSRAT

SEQ ID NO: 120 - protein, 7 aa, synthetic construct
  REGION 1..7: note = Synthetic: CDR-L2, CD39
YASNRAT

SEQ ID NO: 121 - protein, 7 aa, synthetic construct
  REGION 1..7: note = Synthetic: CDR-L2, CD39
WASTRES

SEQ ID NO: 122 - protein, 7 aa, synthetic construct
  REGION 1..7: note = Synthetic: CDR-L2, CD39
WASSRES

SEQ ID NO: 123 - protein, 7 aa, synthetic construct
  REGION 1..7: note = Synthetic: CDR-L2, CD39
WASTRQS

SEQ ID NO: 124 - protein, 7 aa, synthetic construct
  REGION 1..7: note = Synthetic: CDR-L2, CD39
WASTRAS

SEQ ID NO: 125 - protein, 7 aa, synthetic construct
  REGION 1..7: note = Synthetic: CDR-L2, CD39
GASTRAT

SEQ ID NO: 126 - protein, 7 aa, synthetic construct
  REGION 1..7: note = Synthetic: CDR-L2, CD39
GASTRAS

SEQ ID NO: 127 - protein, 7 aa, synthetic construct
  REGION 1..7: note = Synthetic: CDR-L2, CD39
DASNLET

SEQ ID NO: 128 - protein, 7 aa, synthetic construct
  REGION 1..7: note = Synthetic: CDR-L2, CD39
DASNRAT

SEQ ID NO: 129 - protein, 7 aa, synthetic construct
  REGION 1..7: note = Synthetic: CDR-L2, CD39
DASKRAT

SEQ ID NO: 130 - protein, 7 aa, synthetic construct
  REGION 1..7: note = Synthetic: CDR-L2, CD39
KASSLES

SEQ ID NO: 131 - protein, 7 aa, synthetic construct
  REGION 1..7: note = Synthetic: CDR-L2, PD-1 (181)
KVSNRFS

SEQ ID NO: 132 - protein, 7 aa, synthetic construct
  REGION 1..7: note = Synthetic: CDR-L2, PD-1 (Nivolumab)
DASNRAT

SEQ ID NO: 133 - protein, 7 aa, synthetic construct
  REGION 1..7: note = Synthetic: CDR-L2, PD-1 (Pembrolizumab)
LASYLES

SEQ ID NO: 134 - protein, 7 aa, synthetic construct
  REGION 1..7: note = Synthetic: CDR-L2, PD-1 (Cemiplimab)
AASSLHG

SEQ ID NO: 135 - protein, 7 aa, synthetic construct
  REGION 1..7: note = Synthetic: CDR-L2, PD-L1 (Atezolizumab)
SASFLYS

SEQ ID NO: 136 - protein, 7 aa, synthetic construct
  REGION 1..7: note = Synthetic: CDR-L2, PD-L1 (Avelumab)
DVSNRPS

SEQ ID NO: 137 - protein, 7 aa, synthetic construct
  REGION 1..7: note = Synthetic: CDR-L2, PD-L1 (Durvalumab)
DASSRAT

SEQ ID NO: 138 - protein, 8 aa, synthetic construct
  REGION 1..8: note = Synthetic: CDR-L3, CD39
QQYHSYIT

SEQ ID NO: 139 - protein, 8 aa, synthetic construct
  REGION 1..8: note = Synthetic: CDR-L3, CD39
QQYHNAIT

SEQ ID NO: 140 - protein, 8 aa, synthetic construct
  REGION 1..8: note = Synthetic: CDR-L3, CD39
QQYYFYIT

SEQ ID NO: 141 - protein, 8 aa, synthetic construct
  REGION 1..8: note = Synthetic: CDR-L3, CD39
QQYHSALT

SEQ ID NO: 142 - protein, 8 aa, synthetic construct
  REGION 1..8: note = Synthetic: CDR-L3, CD39
QQYHGGIT

SEQ ID NO: 143 - protein, 8 aa, synthetic construct
  REGION 1..8: note = Synthetic: CDR-L3, CD39
QQYHRRIT

SEQ ID NO: 144 - protein, 8 aa, synthetic construct
  REGION 1..8: note = Synthetic: CDR-L3, CD39
QQYHSGIT

SEQ ID NO: 145 - protein, 9 aa, synthetic construct
  REGION 1..9: note = Synthetic: CDR-L3, CD39
QQYYLYPLT

SEQ ID NO: 146 - protein, 9 aa, synthetic construct
  REGION 1..9: note = Synthetic: CDR-L3, CD39
QQYWTYPLT

SEQ ID NO: 147 - protein, 9 aa, synthetic construct
  REGION 1..9: note = Synthetic: CDR-L3, CD39
QQYLLYPLT

SEQ ID NO: 148 - protein, 9 aa, synthetic construct
  REGION 1..9: note = Synthetic: CDR-L3, CD39
QQYLIWPLT

SEQ ID NO: 149 - protein, 9 aa, synthetic construct
  REGION 1..9: note = Synthetic: CDR-L3, CD39
QQYLLWPLT

SEQ ID NO: 150 - protein, 9 aa, synthetic construct
  REGION 1..9: note = Synthetic: CDR-L3, CD39
QQFYFFPPT

SEQ ID NO: 151 - protein, 9 aa, synthetic construct
  REGION 1..9: note = Synthetic: CDR-L3, CD39
QQAYTFPPT

SEQ ID NO: 152 - protein, 9 aa, synthetic construct
  REGION 1..9: note = Synthetic: CDR-L3, CD39
QQYYIFPPT

SEQ ID NO: 153 - protein, 9 aa, synthetic construct
  REGION 1..9: note = Synthetic: CDR-L3, CD39
QQRNFYPPT

SEQ ID NO: 154 - protein, 9 aa, synthetic construct
  REGION 1..9: note = Synthetic: CDR-L3, CD39
QQFVLWPRT

SEQ ID NO: 155 - protein, 9 aa, synthetic construct
  REGION 1..9: note = Synthetic: CDR-L3, CD39
QQHVNFPLT

SEQ ID NO: 156 - protein, 9 aa, synthetic construct
  REGION 1..9: note = Synthetic: CDR-L3, CD39
QQSVFWPIT

SEQ ID NO: 157 - protein, 9 aa, synthetic construct
  REGION 1..9: note = Synthetic: CDR-L3, CD39
QQLTKWPLT

SEQ ID NO: 158 - protein, 9 aa, synthetic construct
  REGION 1..9: note = Synthetic: CDR-L3, CD39
QQDVLWPLT

SEQ ID NO: 159 - protein, 9 aa, synthetic construct
  REGION 1..9: note = Synthetic: CDR-L3, CD39
QQYGLFPIT

SEQ ID NO: 160 - protein, 9 aa, synthetic construct
  REGION 1..9: note = Synthetic: CDR-L3, CD39
QQHTVWPIT

SEQ ID NO: 161 - protein, 9 aa, synthetic construct
  REGION 1..9: note = Synthetic: CDR-L3, CD39
QQVLNYPLT

SEQ ID NO: 162 - protein, 9 aa, synthetic construct
  REGION 1..9: note = Synthetic: CDR-L3, CD39
QQSYFLPPT

SEQ ID NO: 163 - protein, 9 aa, synthetic construct
  REGION 1..9: note = Synthetic: CDR-L3, CD39
QQAHSSPYT

SEQ ID NO: 164 - protein, 9 aa, synthetic construct
  REGION 1..9: note = Synthetic: CDR-L3, PD-1 (181)
FQGSHIPVT

SEQ ID NO: 165 - protein, 9 aa, synthetic construct
  REGION 1..9: note = Synthetic: CDR-L3, PD-1 (Nivolumab)
QQSSNWPRT

SEQ ID NO: 166 - protein, 9 aa, synthetic construct
  REGION 1..9: note = Synthetic: CDR-L3, PD-1 (Pembrolizumab)
QHSRDLPLT

SEQ ID NO: 167 - protein, 9 aa, synthetic construct
  REGION 1..9: note = Synthetic: CDR-L3, PD-1 (Cemiplimab)
QQSSNTPFT

SEQ ID NO: 168 - protein, 9 aa, synthetic construct
  REGION 1..9: note = Synthetic: CDR-L3, PD-L1 (Atezolizumab)
QQYLYHPAT

SEQ ID NO: 169 - protein, 10 aa, synthetic construct
  REGION 1..10: note = Synthetic: CDR-L3, PD-L1 (Avelumab)
SSYTSSSTRV

SEQ ID NO: 170 - protein, 9 aa, synthetic construct
  REGION 1..9: note = Synthetic: CDR-L3, PD-L1 (Durvalumab)
QQYGSLPWT

SEQ ID NO: 171 - protein, 121 aa, synthetic construct
  REGION 1..121: note = Synthetic: VH, CD39
QVQLVQSGAEVKEPGASVKVSCKAPGYTFTSYYMHWVRQAPGQGLEWMGVINPSGGSTSYAQKFQGRVTMTRDTSTSTVYMELSSLRSEDTAVYYCARGKREGGTEYLRHWGQGTLVTVSS

SEQ ID NO: 172 - protein, 121 aa, synthetic construct
  REGION 1..121: note = Synthetic: VH, CD39
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSS

SEQ ID NO: 173 - protein, 121 aa, synthetic construct
  REGION 1..121: note = Synthetic: VH, CD39
QVQLVQSGAEVKKPGASVKVSCKASGYTFTSYQMHWVRQAPGQGLEWMGRINPSGGSTWYAQKFQGRVTMTRDTSTSTVYMELSSLRSEDTAVYYCARGKREGGTEYLRSWGQGTLVTVSS

SEQ ID NO: 174 - protein, 121 aa, synthetic construct
  REGION 1..121: note = Synthetic: VH, CD39
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRNWGQGTLVTVSS

SEQ ID NO: 175 - protein, 121 aa, synthetic construct
  REGION 1..121: note = Synthetic: VH, CD39
QVQLVQSGAEVKKPGASVKVSCKASGYIFKSYEMHWVRQAPGQGLEWMGRINPSVGSTWYAQKFQGRVTMTRDTSTSTVYMELSSLRSEDTAVYYCARGKREGGTEYLRVWGQGTLVTVSS

SEQ ID NO: 176 - protein, 121 aa, synthetic construct
  REGION 1..121: note = Synthetic: VH, CD39
QVQLVQSGAEVKKPGASVKVSCKASGYTFQSYYMHWVRQAPGQGLEWMGKINPSGGSTWYAQKFQGRVTMTRDTSTSTVYMELSSLRSEDTAVYYCARGKREGGTEYLRHWGQGTLVTVSS

SEQ ID NO: 177 - protein, 121 aa, synthetic construct
  REGION 1..121: note = Synthetic: VH, CD39
QVQLVQSGAEVKKPGASVKVSCKASGYTFKSYEMHWVRQAPGQGLEWMGRINPSGGSTWYAQKFQGRVTMTRDTSTSTVYMELSSLRSEDTAVYYCARGKREGGTEYLRHWGQGTLVTVSS

SEQ ID NO: 178 - protein, 121 aa, synthetic construct
  REGION 1..121: note = Synthetic: VH, CD39
QVQLVQSGAEVKKPGASVKVSCKASGYTFTSYQMHWVRQAPGQGLEWMGRINPSGGSTWYAQKFQGRVTMTRDTSTSTVYMELSSLRSEDTAVYYCARGKREGGTEYLRHWGQGTLVTVSS

SEQ ID NO: 179 - protein, 121 aa, synthetic construct
  REGION 1..121: note = Synthetic: VH, CD39
QVQLVQSGAEVKKPGASVKVSCKASGYTFFSYYMYWVRQAPGQGLEWMGVINPLGGGTSYAQKFQGRVTMTRDTSTSTVYMELSSLRSEDTAVYYCARGKREGGTEYLRHWGQGTLVTVSS

SEQ ID NO: 180 - protein, 121 aa, synthetic construct
  REGION 1..121: note = Synthetic: VH, CD39
QVQLVQSGAEVKKPGASVKVSCKASGYTFVSYFMHWVRQAPGQGLEWMGSINPRGGSTSYAQKFQGRVTMTRDTSTSTVYMELSSLRSEDTAVYYCARGKREGGTEYLRHWGQGTLVTVSS

SEQ ID NO: 181 - protein, 121 aa, synthetic construct
  REGION 1..121: note = Synthetic: VH, CD39
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRHWGQGTLVTVSS

SEQ ID NO: 182 - protein, 122 aa, synthetic construct
  REGION 1..122: note = Synthetic: VH, CD39
QVQLVQSGAEVKKPGSSVKVSCKASGGTFSSLAISWVRQAPGQGLEWMGGIIPIFGTANYAQKFQGRVTITADESTNTAYMELSSLRSEDTAVYYCARGGAKYASTYGMDVWGQGTTVTVSS

SEQ ID NO: 183 - protein, 120 aa, synthetic construct
  REGION 1..120: note = Synthetic: VH, CD39
QVQLVQSGAEVKKPGSSVKVSCKASGGTFSKLAISWVRQAPGQGLEWMGGIGFGTANYAQKFQGRVTITADESASTAYMELSSLRSEDTAVYYCARGGAKYASTHGMDVWGQGTTVTVSS

SEQ ID NO: 184 - protein, 122 aa, synthetic construct
  REGION 1..122: note = Synthetic: VH, CD39
QVQLVQSGAEVKKPGSSVKVSCKASGGTFSHTAISWVRQAPGQGLEWMGGILPIGGTANYAQKFQGRVTITADESTSTAYMELSSLRSEDTAVYYCARGGAKYASQLGMDVWGQGTTVTVSS

SEQ ID NO: 185 - protein, 120 aa, synthetic construct
  REGION 1..120: note = Synthetic: VH, CD39
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYASKWGMDVWGQGTTVTVSS

SEQ ID NO: 186 - protein, 122 aa, synthetic construct
  REGION 1..122: note = Synthetic: VH, CD39
QVQLVQSGAEVKKPGSSVKVSCKASGGTFSLLAISWVRQAPGQGLEWMGGILPIAGTANYAQKFQGRVTITADESTSTAYMELSSLRSEDTAVYYCARGGAKYAVGYGMDVWGQGTTVTVSS

SEQ ID NO: 187 - protein, 122 aa, synthetic construct
  REGION 1..122: note = Synthetic: VH, CD39
QVQLVQSGAEVKKPGASVKVSCKASGGTFQSLAISWVRQAPGQGLEWMGGILPIGGTANYAQKFQGRVTITADESTSTAYMELSSLRSEDTAVYYCARGGAKYAGRYGMDVWGQGTTVTVSS

SEQ ID NO: 188 - protein, 120 aa, synthetic construct
  REGION 1..120: note = Synthetic: VH, CD39
QVQLVQSGAEVKKPGSSVKVSCKASGGTFPSNAISWVRQAPGQGLEWMGGIGFGTANYAQKFQGRVTITADESTSTAYMELSSLRSEDTAVYYCARGGAKYARTYGMDVWGQGTTVTVSS

SEQ ID NO: 189 - protein, 120 aa, synthetic construct
  REGION 1..120: note = Synthetic: VH, CD39
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYAGRYGMDVWGQGTTVTVSS

SEQ ID NO: 190 - protein, 122 aa, synthetic construct
  REGION 1..122: note = Synthetic: VH, CD39
QVQLVQSGAEVKKPGSSVKVSCKASGGTFSAMAISWVRQAPGQGLEWMGGILPIAGTANYAQKFQGRVTITADESTSTAYMELSSLRSEDTAVYYCARGGAKYASTYGMDVWGQGTTVTVSS

SEQ ID NO: 191 - protein, 122 aa, synthetic construct
  REGION 1..122: note = Synthetic: VH, CD39
QVQLVQSGAEVKKPGSSVKVSCKASGGTFASLAISWVRQAPGQGLEWMGGILPIFGEANYAQKFQGRVTITADESTSTAYMELSSLRSEDTAVYYCARGGAKYASTYGMDVWGQGTTVTVSS

SEQ ID NO: 192 - protein, 122 aa, synthetic construct
  REGION 1..122: note = Synthetic: VH, CD39
QVQLVQSGAEVKKPGSSVKVSCKASGGTFSWLAISWVRQAPGQGLEWMGGIIPRGGTANYAQKFQGRVTITADESTSTAYMELSSLRSEDTAVYYCARGGAKYASTYGMDVWGQGTTVTVSS

SEQ ID NO: 193 - protein, 121 aa, synthetic construct
  REGION 1..121: note = Synthetic: VH, CD39
QVQLVQSGAEVKKPGSSVKASCKASGGTFSSYAISWVRQAPGQGLEWMGSIIPIFGTANYAQKFRGRVTITADESTSTTYMELSSLRSEDTAVYYCARESGGYRDHRLDVWGQGTMVTVSS

SEQ ID NO: 194 - protein, 121 aa, synthetic construct
  REGION 1..121: note = Synthetic: VH, CD39
QVQLVQSGAEVKKPGSSVKVSCKASGGTFGSYGISWVRQAPGQGLEWMGSIIPEFGIANYAQKFQGRVTITADESTSTAYMELSSLRSEDTAVYYCARESGTYRDHRLDVWGQGTMVTVSS

SEQ ID NO: 195 - protein, 121 aa, synthetic construct
  REGION 1..121: note = Synthetic: VH, CD39
QVQLVQSGAEVKKPGSSVKVSCKASGGTFSKYGISWVRQAPGQGLEWMGSIIPEFGIANYAQKFQGRVTITADESTSTAYMELSSLRSEDTAVYYCARESGGYRDHRLGVWGQGTMVTVSS

SEQ ID NO: 196 - protein, 121 aa, synthetic construct
  REGION 1..121: note = Synthetic: VH, CD39
QVQLVQSGAEVKKPGSSVKVSCKASGGTFESYGISWVRQAPGQGLEWMGSIIPEFGIANYAQKFQGRVTITADESTSTTYMELSSLRSEDTAVYYCARESGGYRDHRLDVWGQGTMVTVSS

SEQ ID NO: 197 - protein, 124 aa, synthetic construct
  REGION 1..124: note = Synthetic: VH, CD39
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDFTDYSSGYSSGWTYWGQGTLVTVSS

SEQ ID NO: 198 - protein, 123 aa, synthetic construct
  REGION 1..123: note = Synthetic: VH, CD39
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARDTLYSSGAYYGYNVWGQGTMVTVSS

SEQ ID NO: 199 - protein, 124 aa, synthetic construct
  REGION 1..124: note = Synthetic: VH, CD39
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARAKRGYDSYGGVYFDYWGQGTLVTVSS

SEQ ID NO: 200 - protein, 127 aa, synthetic construct
  REGION 1..127: note = Synthetic: VH, CD39
QVQLVQSGAEVKKPGSSVKVSCKASGGTFSNYAISWVRQAPGQGLEWMGGIIPIFGTANYAQKFQGRVTITADESTSTAYMELSSLRSEDTAVYYCARGPTVTATTSIGTHNWFDPWGQGTLVTVSS

SEQ ID NO: 201 - protein, 126 aa, synthetic construct
  REGION 1..126: note = Synthetic: VH, CD39
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCAREGRGYDSSRYYKFWFDPWGQGTLVTVSS

SEQ ID NO: 202 - protein, 121 aa, synthetic construct
  REGION 1..121: note = Synthetic: VH, CD39
QVQLVQSGAEVKEPGSSVKVSCKASGGTFSSYATSWVRQAPGQGLEWMGGIIPISGTANYAQEFQGRVTITADESTSTAYMELSSLRSEDTAVYYCARDGGGYRHHYFDLWGRGTLVTVSS

SEQ ID NO: 203 - protein, 121 aa, synthetic construct
  REGION 1..121: note = Synthetic: VH, CD39
QVQLVQSGAEVKKPGSSVKVPCKASGGTFSSYAISWVRQAPEQGLEWMGSIIPIFGTANYAQKFQGRVTITADESTSTAYMELSSLRSEDTAVYYCAGESGGYRDHKLDVWGQGTVVTVSS

SEQ ID NO: 204 - protein, 121 aa, synthetic construct
  REGION 1..121: note = Synthetic: VH, CD39
QVQLVQSGAEVKKPGSSVKVSCKASGGAFSSYAIGWVRQAPGQGLEWMGGIIPTFGTANYAQKFQGRVTITADESTSTAYMELSSLRSEDTAVYYCARDGGGYQHHYFDLWGRGTLVTVSS

SEQ ID NO: 205 - protein, 121 aa, synthetic construct
  REGION 1..121: note = Synthetic: VH, CD39
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARESGGYRDHKLDVWGQGTMVTVSS

SEQ ID NO: 206 - protein, 120 aa, synthetic construct
  REGION 1..120: note = Synthetic: VH, CD39
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDSGYHRHYSDYWGQGTLVTVSS

SEQ ID NO: 207 - protein, 121 aa, synthetic construct
  REGION 1..121: note = Synthetic: VH, CD39
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPLGIRKHWFDPWGQGTLVTVSS

SEQ ID NO: 208 - protein, 121 aa, synthetic construct
  REGION 1..121: note = Synthetic: VH, CD39
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDTPRWRYHYFDYWGQGTLVTVSS

SEQ ID NO: 209 - protein, 121 aa, synthetic construct
  REGION 1..121: note = Synthetic: VH, CD39
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARERRGSLALGMDVWGQGTLVTVSS

SEQ ID NO: 210 - protein, 128 aa, synthetic construct
  REGION 1..128: note = Synthetic: VH, CD39
QVQLVESGGGVVQPGRSLRLSCAASGFTFSSYGMNWVRQAPGKGLEWVAVIWYDGSNKYYADSVKGRFTISRDNSKNTLYLQMNSLRAEDTAVYYCARDLGGYSYGEPYYYYYGMDVWGQGTTVTVSS

SEQ ID NO: 211 - protein, 118 aa, synthetic construct
  REGION 1..118: note = Synthetic: VH, PD-1 (181)
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSS

SEQ ID NO: 212 - protein, 113 aa, synthetic construct [sequence omitted]

SEQ ID NO: 213 - protein, 120 aa, synthetic construct [sequence omitted]

SEQ ID NO: 214 - protein, 117 aa, synthetic construct [sequence omitted]

SEQ ID NO: 215 - protein, 118 aa, synthetic construct [sequence omitted]

SEQ ID NO: 216 - protein, 120 aa, synthetic construct
  REGION 1..120: note = Synthetic: VH, PD-L1 (Avelumab)
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 217 - protein, 121 aa, synthetic construct
  REGION 1..121: note = Synthetic: VH, PD-L1 (Durvalumab)
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 218 - protein, 107 aa, synthetic construct
  REGION 1..107: note = Synthetic: VL, CD39
EIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIK

SEQ ID NO: 219 - protein, 107 aa, synthetic construct
  REGION 1..107: note = Synthetic: VL, CD39
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIK

SEQ ID NO: 220 - protein, 107 aa, synthetic construct
  REGION 1..107: note = Synthetic: VL, CD39
EIVLTQSPGTLSLSPGERATLSCRASQSVSSSYLAWYQQKPGQAPRLLIYYASSRAYGIPDRFSGSGSGTDFTLTISRLEPEDFAVYYCQQYHNAITFGGGTKVEIK

SEQ ID NO: 221 - protein, 107 aa, synthetic construct
  REGION 1..107: note = Synthetic: VL, CD39
EIVLTQSPGTLSLSPGERATLSCRASQSVSSSYLAWYQQKPGQAPRLLIYGASSRATGIPDRFSGSGSGTDFTLTISRLEPEDFAVYYCQQYYFYITFGGGTKVEIK

SEQ ID NO: 222 - protein, 107 aa, synthetic construct
  REGION 1..107: note = Synthetic: VL, CD39
EIVLTQSPGTLSLSPGERATLSCEASQSVSYSYLAWYQQKPGQAPRLLIYGASSRANGIPDRFSGSGSGTDFTLTISRLEPEDFAVYYCQQYHSALTFGGGTKVEIK

SEQ ID NO: 223 - protein, 107 aa, synthetic construct
  REGION 1..107: note = Synthetic: VL, CD39
EIVLTQSPGTLSLSPGERATLSCRASQSVASSYLAWYQQKPGQAPRLLIYGASNRHTGIPDRFSGSGSGTDFTLTISRLEPEDFAVYYCQQYHGGITFGGGTKVEIK

SEQ ID NO: 224 - protein, 107 aa, synthetic construct
  REGION 1..107: note = Synthetic: VL, CD39
EIVLTQSPGTLSLSPGERATLSCKASESVSSSYLAWYQQKPGQAPRLLIYYASSRATGIPDRFSGSGSGTDFTLTISRLEPEDFAVYYCQQYHRRITFGGGTKVEIK

SEQ ID NO: 225 - protein, 107 aa, synthetic construct
  REGION 1..107: note = Synthetic: VL, CD39
EIVLTQSPGTLSLSPGERATLSCRASQYVSSSYLAWYQQKPGQAPRLLIYYASNRATGIPDRFSGSGSGTDFTLTISRLEPEDFAVYYCQQYHSGITFGGGTKVEIK

SEQ ID NO: 226 - protein, 113 aa, synthetic construct
  REGION 1..113: note = Synthetic: VL, CD39
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYLYPLTFGGGTKVEIK

SEQ ID NO: 227 - protein, 113 aa, synthetic construct
  REGION 1..113: note = Synthetic: VL, CD39
DIVMTQSPDSLAVSLGERATINCKSSRSVLFSSNNKNYLAWYQQKPGQPPKLLIYWASTRESGVPDRFSGSGSGTDFTLTISSLQAEDVAVYYCQQYWTYPLTFGGGTKVEIK

SEQ ID NO: 228 - protein, 113 aa, synthetic construct
  REGION 1..113: note = Synthetic: VL, CD39
DIVMTQSPDSLAVSLGERATINCKSSQSVLFSSNNKNYLAWYQQKPGQPPKLLIYWASSRESGVPDRFSGSGSGTDFTLTISSLQAEDVAVYYCQQYWTYPLTFGGGTKVEIK

SEQ ID NO: 229 - protein, 113 aa, synthetic construct
  REGION 1..113: note = Synthetic: VL, CD39
DIVMTQSPDSLAVSLGERATINCKSSKSVLYSNNNKNYLAWYQQKPGQPPKLLIYWASTRQSGVPDRFSGSGSGTDFTLTISSLQAEDVAVYYCQQYLLYPLTFGGGTKVEIK

SEQ ID NO: 230 - protein, 113 aa, synthetic construct
  REGION 1..113: note = Synthetic: VL, CD39
GIVMTQSPDSLAVSLGERATINCKSSQSVLFSSNNKNYLAWYQQKPGQPPKLLIYWASTRASGVPDRFSGSGSGTDFTLTISSLQAEDVAVYYCQQYYLYPLTFGGGTKVEIK

SEQ ID NO: 231 - protein, 107 aa, synthetic construct
  REGION 1..107: note = Synthetic: VL, CD39
EIVMTQSPATLSVSPGERATLSCRASQSVGSNLAWYQQKPGQAPRLLIYGASTRATGIPARFSGSGSGTEFTLTISSLQSEDFAVYYCQQYLIWPLTFGGGTKVEIK

SEQ ID NO: 232 - protein, 107 aa, synthetic construct
  REGION 1..107: note = Synthetic: VL, CD39
EIVMTQSPATLSVSPGERATLSCRASQSVGSNLAWYQQKPGQAPRLLIYGASTRATGIPARFSGSGSGTEFTLTISSLQSEDFAVYYCQQYLLWPLTFGGGTKVEIK

SEQ ID NO: 233 - protein, 107 aa, synthetic construct
  REGION 1..107: note = Synthetic: VL, CD39
EIVMTQSPATLSVSPGERATLSCRASQSVGSNLAWYQQKPGQAPRLLIYGASTRASGIPARFSGSGSGTEFTLTISSLQSEDFAVYYCQQYLLWPLTFGGGTKVEIK

SEQ ID NO: 234 - protein, 113 aa, synthetic construct
  REGION 1..113: note = Synthetic: VL, CD39
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQFYFYPPTFGGGTKVEIK

SEQ ID NO: 235 - protein, 113 aa, synthetic construct
  REGION 1..113: note = Synthetic: VL, CD39
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQAYTFPPTFGGGTKVEIK

SEQ ID NO: 236 - protein, 107 aa, synthetic construct
  REGION 1..107: note = Synthetic: VL, CD39
DIQMTQSPSSLSASVGDRVTITCQASQDISNYLNWYQQKPGKAPKLLIYDASNLETGVPSRFSGSGSGTDFTFTISSLQPEDIATYYCQQYYIFPPTFGGGTKVEIK

SEQ ID NO: 237 - protein, 107 aa, synthetic construct
  REGION 1..107: note = Synthetic: VL, CD39
EIVLTQSPATLSLSPGERATLSCRASQSVSSYLAWYQQKPGQAPRLLIYDASNRATGIPARFSGSGSGTDFTLTISSLEPEDFAVYYCQQRNFYPPTFGGGTKVEIK

SEQ ID NO: 238 - protein, 107 aa, synthetic construct
  REGION 1..107: note = Synthetic: VL, CD39
EIVLTQSPATLSLSPGERATLSCRASQSVSSYLAWYQQKPGQAPRLLIYDASNRATGIPARFSGSGSGTDFTLTISSLEPEDFAVYYCQQFVLWPRTFGGGTKVEIK

SEQ ID NO: 239 - protein, 107 aa, synthetic construct
  REGION 1..107: note = Synthetic: VL, CD39
EIVLTQSPATLSLSPGERATLSCRASQSVSRYLAWYQQKPGQAPRLLIYDASNRATGIPARFSGSGSGTDFTLTISSLEPEDFAVYYCQQHVNFPLTFGGGTKVEIK

SEQ ID NO: 240 - protein, 107 aa, synthetic construct
  REGION 1..107: note = Synthetic: VL, CD39
EIVLTQSPATLSLSPGERATLSCRASQSVSSYLAWYQQKPGQAPRLLIYDASNRATGIPARFSGSGSGTDFTLTISSLEPEDFAVYYCQQSVFWPITFGGGTKVEIK

SEQ ID NO: 241 - protein, 107 aa, synthetic construct
  REGION 1..107: note = Synthetic: VL, CD39
EIVMTQSPATLSVSPGERATLSCRASQSVGSNLAWYQQKPGQAPRLLIYGASTRATGIPARFSGSGSGTEFTLTISSLQSEDFAVYYCQQLTKWPLTFGGGTKVEIK

SEQ ID NO: 242 - protein, 107 aa, synthetic construct
  REGION 1..107: note = Synthetic: VL, CD39
EIVLTQSPATLSLSPGERATLSCRASQSVSSYLAWYQQKPGQAPRLLIYDASKRATGIPARFSGSGSGTDFTLTISSLEPEDFAVYYCQQDVLWPLTFGGGTKVEIK

SEQ ID NO: 243 - protein, 107 aa, synthetic construct
  REGION 1..107: note = Synthetic: VL, CD39
DIQMTQSPSTLSASVGDRVTITCRASQSISSWLAWYQQKPGKAPKLLIYKASSLESGVPSRFSGSGSGTEFTLTISSLQPDDFATYYCQQYGLFPITFGGGTKVEIK

SEQ ID NO: 244 - protein, 107 aa, synthetic construct
  REGION 1..107: note = Synthetic: VL, CD39
EIVMTQSPATLSLSPGERATLSCRASQSVSSYLAWYQQKPGQAPRLLIYDASNRATGIPARFSGSGSGTDFTLTISSLEPEDFAVYYCQQHTVWPITFGGGTKVEIK

SEQ ID NO: 245 - protein, 107 aa, synthetic construct
  REGION 1..107: note = Synthetic: VL, CD39
EIVLTQSPATLSLSPGERATLSCRASQSVSSYLAWYQQKPGQAPRLLIYDASKRATGIPARFSGSGSGTDFTLTISSLEPEDFAVYYCQQVLNYPLTFGGGTKVEIK

SEQ ID NO: 246 - protein, 107 aa, synthetic construct
  REGION 1..107: note = Synthetic: VL, CD39
DIQMTQSPSSLSASVGDRVTITCQASQDISNYLNWYQQKPGKAPKLLIYDASNLETGVPSRFSGSGSGTDFTFTISSLQPEDIATYYCQQSYFLPPTFGGGTKVEIK

SEQ ID NO: 247 - protein, 108 aa, synthetic construct
  REGION 1..108: note = Synthetic: VL, CD39
EIVLTQSPGTLSLSPGERATLSCRASQSVSSDYLAWYQQKPGQAPRLLIYGASSRATGIPDRFSGSGSGTDFTLTISRLEPEDFAVYYCQQAHSSPYTFGGGTKVEIK

SEQ ID NO: 248 - protein, 112 aa, synthetic construct
  REGION 1..112: note = Synthetic: VL, PD-1 (181)
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIK

SEQ ID NO: 249 - protein, 107 aa, synthetic construct [sequence omitted]

SEQ ID NO: 250 - protein, 111 aa, synthetic construct [sequence omitted]

SEQ ID NO: 251 - protein, 107 aa, synthetic construct [sequence omitted]

SEQ ID NO: 252 - protein, 107 aa, synthetic construct [sequence omitted]

SEQ ID NO: 253 - protein, 106 aa, synthetic construct
  REGION 1..106: note = Synthetic: VL, PD-L1 (Avelumab)
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTK

SEQ ID NO: 254 - protein, 108 aa, synthetic construct
  REGION 1..108: note = Synthetic: VL, PD-L1 (Durvalumab)
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 255 - protein, 448 aa, synthetic construct
  REGION 1..448: note = Synthetic: HC, CD39
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 256 - protein, 214 aa, synthetic construct
  REGION 1..214: note = Synthetic: LC, CD39
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 448 aa, synthetic construct
  REGION 1..448: note = Synthetic: HC, CD39
QVQLVQSGAEVKKPGASVKVSCKASGYTFTSYQMHWVRQAPGQGLEWMGRINPSGGSTWYAQKFQGRVTMTRDTSTSTVYMELSSLRSEDTAVYYCARGKREGGTEYLRSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 258 - protein, 214 aa, synthetic construct
  REGION 1..214: note = Synthetic: LC, CD39
EIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - protein, 448 aa, synthetic construct
  REGION 1..448: note = Synthetic: HC, CD39
QVQLVQSGAEVKKPGASVKVSCKASGYIFKSYEMHWVRQAPGQGLEWMGRINPSVGSTWYAQKFQGRVTMTRDTSTSTVYMELSSLRSEDTAVYYCARGKREGGTEYLRVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 260 - protein, 214 aa, synthetic construct
  REGION 1..214: note = Synthetic: LC, CD39
EIVLTQSPGTLSLSPGERATLSCRASQSVSSSYLAWYQQKPGQAPRLLIYYASSRAYGIPDRFSGSGSGTDFTLTISRLEPEDFAVYYCQQYHNAITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 448 aa, synthetic construct
  REGION 1..448: note = Synthetic: HC, CD39
QVQLVQSGAEVKKPGASVKVSCKASGYTFKSYEMHWVRQAPGQGLEWMGRINPSGGSTWYAQKFQGRVTMTRDTSTSTVYMELSSLRSEDTAVYYCARGKREGGTEYLRHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 262 - protein, 214 aa, synthetic construct
  REGION 1..214: note = Synthetic: LC, CD39
EIVLTQSPGTLSLSPGERATLSCRASQSVSSSYLAWYQQKPGQAPRLLIYGASSRATGIPDRFSGSGSGTDFTLTISRLEPEDFAVYYCQQYYFYITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 448 aa, synthetic construct; identical to SEQ ID NO: 261
  REGION 1..448: note = Synthetic: HC, CD39

SEQ ID NO: 264 - protein, 214 aa, synthetic construct
  REGION 1..214: note = Synthetic: LC, CD39
EIVLTQSPGTLSLSPGERATLSCEASQSVSYSYLAWYQQKPGQAPRLLIYGASSRANGIPDRFSGSGSGTDFTLTISRLEPEDFAVYYCQQYHSALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 447 aa, synthetic construct
  REGION 1..447: note = Synthetic: HC, CD39
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYASKWGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 266 - protein, 220 aa, synthetic construct
  REGION 1..220: note = Synthetic: LC, CD39
DIVMTQSPDSLAVSLGERATINCKSSQSVLFSSNNKNYLAWYQQKPGQPPKLLIYWASSRESGVPDRFSGSGSGTDFTLTISSLQAEDVAVYYCQQYWTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 447 aa, synthetic construct
  REGION 1..447: note = Synthetic: HC, CD39
QVQLVQSGAEVKKPGSSVKVSCKASGGTFPSNAISWVRQAPGQGLEWMGGIGFGTANYAQKFQGRVTITADESTSTAYMELSSLRSEDTAVYYCARGGAKYARTYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 268 - protein, 220 aa, synthetic construct
  REGION 1..220: note = Synthetic: LC, CD39
DIVMTQSPDSLAVSLGERATINCKSSKSVLYSNNNKNYLAWYQQKPGQPPKLLIYWASTRQSGVPDRFSGSGSGTDFTLTISSLQAEDVAVYYCQQYLLYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 447 aa, synthetic construct
  REGION 1..447: note = Synthetic: HC, CD39
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYAGRYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 270 - protein, 220 aa, synthetic construct
  REGION 1..220: note = Synthetic: LC, CD39
GIVMTQSPDSLAVSLGERATINCKSSQSVLFSSNNKNYLAWYQQKPGQPPKLLIYWASTRASGVPDRFSGSGSGTDFTLTISSLQAEDVAVYYCQQYYLYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 448 aa, synthetic construct
  REGION 1..448: note = Synthetic: HC, CD39
QVQLVQSGAEVKKPGSSVKVSCKASGGTFGSYGISWVRQAPGQGLEWMGSIIPEFGIANYAQKFQGRVTITADESTSTAYMELSSLRSEDTAVYYCARESGTYRDHRLDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 272 - protein, 214 aa, synthetic construct
  REGION 1..214: note = Synthetic: LC, CD39
EIVMTQSPATLSVSPGERATLSCRASQSVGSNLAWYQQKPGQAPRLLIYGASTRATGIPARFSGSGSGTEFTLTISSLQSEDFAVYYCQQYLLWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 448 aa, synthetic construct
  REGION 1..448: note = Synthetic: HC, CD39
QVQLVQSGAEVKKPGSSVKVSCKASGGTFSKYGISWVRQAPGQGLEWMGSIIPEFGIANYAQKFQGRVTITADESTSTAYMELSSLRSEDTAVYYCARESGGYRDHRLGVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 274 - protein, 214 aa, synthetic construct
  REGION 1..214: note = Synthetic: LC, CD39
EIVMTQSPATLSVSPGERATLSCRASQSVGSNLAWYQQKPGQAPRLLIYGASTRASGIPARFSGSGSGTEFTLTISSLQSEDFAVYYCQQYLLWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 275 - protein, 448 aa, synthetic construct
  REGION 1..448: note = Synthetic: HC, CD39
QVQLVQSGAEVKKPGSSVKVSCKASGGTFESYGISWVRQAPGQGLEWMGSIIPEFGIANYAQKFQGRVTITADESTSTTYMELSSLRSEDTAVYYCARESGGYRDHRLDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 276 - protein, 214 aa, synthetic construct; identical to SEQ ID NO: 272
  REGION 1..214: note = Synthetic: LC, CD39

SEQ ID NO: 277 - protein, 453 aa, synthetic construct
  REGION 1..453: note = Synthetic: HC, CD39
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCAREGRGYDSSRYYKFWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 278 - protein, 214 aa, synthetic construct
  REGION 1..214: note = Synthetic: LC, CD39
EIVLTQSPATLSLSPGERATLSCRASQSVSSYLAWYQQKPGQAPRLLIYDASNRATGIPARFSGSGSGTDFTLTISSLEPEDFAVYYCQQFVLWPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 448 aa, synthetic construct
  REGION 1..448: note = Synthetic: HC, CD39
QVQLVQSGAEVKEPGSSVKVSCKASGGTFSSYATSWVRQAPGQGLEWMGGIIPISGTANYAQEFQGRVTITADESTSTAYMELSSLRSEDTAVYYCARDGGGYRHHYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 280 - protein, 214 aa, synthetic construct
  REGION 1..214: note = Synthetic: LC, CD39
EIVLTQSPATLSLSPGERATLSCRASQSVSRYLAWYQQKPGQAPRLLIYDASNRATGIPARFSGSGSGTDFTLTISSLEPEDFAVYYCQQHVNFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 448 aa, synthetic construct
  REGION 1..448: note = Synthetic: HC, CD39
QVQLVQSGAEVKKPGSSVKVPCKASGGTFSSYAISWVRQAPEQGLEWMGSIIPIFGTANYAQKFQGRVTITADESTSTAYMELSSLRSEDTAVYYCAGESGGYRDHKLDVWGQGTVVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 282 - protein, 214 aa, synthetic construct
  REGION 1..214: note = Synthetic: LC, CD39
EIVLTQSPATLSLSPGERATLSCRASQSVSSYLAWYQQKPGQAPRLLIYDASNRATGIPARFSGSGSGTDFTLTISSLEPEDFAVYYCQQSVFWPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 448 aa, synthetic construct
  REGION 1..448: note = Synthetic: HC, CD39
QVQLVQSGAEVKKPGSSVKVSCKASGGAFSSYAIGWVRQAPGQGLEWMGGIIPTFGTANYAQKFQGRVTITADESTSTAYMELSSLRSEDTAVYYCARDGGGYQHHYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 284 - protein, 214 aa, synthetic construct
  REGION 1..214: note = Synthetic: LC, CD39
EIVMTQSPATLSVSPGERATLSCRASQSVGSNLAWYQQKPGQAPRLLIYGASTRATGIPARFSGSGSGTEFTLTISSLQSEDFAVYYCQQLTKWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 285 - protein, 448 aa, synthetic construct
  REGION 1..448: note = Synthetic: HC, CD39
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARESGGYRDHKLDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 286 - protein, 214 aa, synthetic construct
  REGION 1..214: note = Synthetic: LC, CD39
EIVLTQSPATLSLSPGERATLSCRASQSVSSYLAWYQQKPGQAPRLLIYDASKRATGIPARFSGSGSGTDFTLTISSLEPEDFAVYYCQQDVLWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - protein, 447 aa, synthetic construct
  REGION 1..447: note = Synthetic: HC, CD39
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDSGYHRHYSDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 288 - protein, 214 aa, synthetic construct
  REGION 1..214: note = Synthetic: LC, CD39
DIQMTQSPSTLSASVGDRVTITCRASQSISSWLAWYQQKPGKAPKLLIYKASSLESGVPSRFSGSGSGTEFTLTISSLQPDDFATYYCQQYGLFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - protein, 448 aa, synthetic construct
  REGION 1..448: note = Synthetic: HC, CD39
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPLGIRKHWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 290 - protein, 214 aa, synthetic construct
  REGION 1..214: note = Synthetic: LC, CD39
EIVMTQSPATLSLSPGERATLSCRASQSVSSYLAWYQQKPGQAPRLLIYDASNRATGIPARFSGSGSGTDFTLTISSLEPEDFAVYYCQQHTVWPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 291 - protein, 448 aa, synthetic construct
  REGION 1..448: note = Synthetic: HC, CD39
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDTPRWRYHYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 292 - protein, 214 aa, synthetic construct
  REGION 1..214: note = Synthetic: LC, CD39
EIVLTQSPATLSLSPGERATLSCRASQSVSSYLAWYQQKPGQAPRLLIYDASKRATGIPARFSGSGSGTDFTLTISSLEPEDFAVYYCQQVLNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 293 - protein, 448 aa, synthetic construct
  REGION 1..448: note = Synthetic: HC, CD39
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARERRGSLALGMDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 294 - protein, 214 aa, synthetic construct
  REGION 1..214: note = Synthetic: LC, CD39
DIQMTQSPSSLSASVGDRVTITCQASQDISNYLNWYQQKPGKAPKLLIYDASNLETGVPSRFSGSGSGTDFTFTISSLQPEDIATYYCQQSYFLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 455 aa, synthetic construct
  REGION 1..455: note = Synthetic: HC, CD39
QVQLVESGGGVVQPGRSLRLSCAASGFTFSSYGMNWVRQAPGKGLEWVAVIWYDGSNKYYADSVKGRFTISRDNSKNTLYLQMNSLRAEDTAVYYCARDLGGYSYGEPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 296 - protein, 215 aa, synthetic construct
  REGION 1..215: note = Synthetic: LC, CD39
EIVLTQSPGTLSLSPGERATLSCRASQSVSSDYLAWYQQKPGQAPRLLIYGASSRATGIPDRFSGSGSGTDFTLTISRLEPEDFAVYYCQQAHSSPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - protein, 448 aa, synthetic construct
  REGION 1..448: note = Synthetic: HC, CD39
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 298 - protein, 214 aa, synthetic construct; identical to SEQ ID NO: 258
  REGION 1..214: note = Synthetic: LC, CD39

SEQ ID NO: 299 - protein, 448 aa, synthetic construct
  REGION 1..448: note = Synthetic: HC, PD-1 (181)
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 300 - protein, 447 aa, synthetic construct
  REGION 1..447: note = Synthetic: HC, PD-1 (181)
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 301 - protein, 219 aa, synthetic construct
  REGION 1..219: note = Synthetic: LC, PD-1 (181)
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 302 - protein, 440 aa, synthetic construct
  REGION 1..440: note = Synthetic: HC, PD-1 (Nivolumab)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 303 - protein, 214 aa, synthetic construct
  REGION 1..214: note = Synthetic: LC, PD-1 (Nivolumab)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 304 - protein, 447 aa, synthetic construct
  REGION 1..447: note = Synthetic: HC, PD-1 (Pembrolizumab)
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 305 - protein, 218 aa, synthetic construct
  REGION 1..218: note = Synthetic: LC, PD-1 (Pembrolizumab)
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 306 - protein, 444 aa, synthetic construct
  REGION 1..444: note = Synthetic: HC, PD-1 (Cemiplimab)
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 307 - protein, 214 aa, synthetic construct
  REGION 1..214: note = Synthetic: LC, PD-1 (Cemiplimab)
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 308 - protein, 448 aa, synthetic construct
  REGION 1..448: note = Synthetic: HC, PD-L1 (Atezolizumab)
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 214 aa, synthetic construct
  REGION 1..214: note = Synthetic: LC, PD-L1 (Atezolizumab)
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 310 - protein, 450 aa, synthetic construct
  REGION 1..450: note = Synthetic: HC, PD-L1 (Avelumab)
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 216 aa, synthetic construct
  REGION 1..216: note = Synthetic: LC, PD-L1 (Avelumab)
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 312 - protein, 451 aa, synthetic construct
  REGION 1..451: note = Synthetic: HC, PD-L1 (Durvalumab)
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 215 aa, synthetic construct
  REGION 1..215: note = Synthetic: LC, PD-L1 (Durvalumab)
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 314 - skipped

SEQ ID NO: 315 - protein, 5 aa, synthetic construct
  REGION 1..5: note = Synthetic: CDR-H1, CD39
SYRMN

SEQ ID NO: 316 - protein, 5 aa, synthetic construct
  REGION 1..5: note = Synthetic: CDR-H1, CD39
DKAIS

SEQ ID NO: 317 - protein, 5 aa, synthetic construct
  REGION 1..5: note = Synthetic: CDR-H1, CD39
SEGIS

SEQ ID NO: 318 - protein, 5 aa, synthetic construct
  REGION 1..5: note = Synthetic: CDR-H1, CD39
TYAIG

SEQ ID NO: 319 - protein, 5 aa, synthetic construct
  REGION 1..5: note = Synthetic: CDR-H1, CD39
SWYMH

SEQ ID NO: 320 - skipped

SEQ ID NO: 321 - protein, 17 aa, synthetic construct
  REGION 1..17: note = Synthetic: CDR-H2, CD39
SISSSSSSIWYADSVKG

SEQ ID NO: 322 - protein, 17 aa, synthetic construct
  REGION 1..17: note = Synthetic: CDR-H2, CD39
SILPIFGTANYAQKFQG

SEQ ID NO: 323 - protein, 17 aa, synthetic construct
  REGION 1..17: note = Synthetic: CDR-H2, CD39
SILPIFGTANYAQKFQG

SEQ ID NO: 324 - protein, 17 aa, synthetic construct
  REGION 1..17: note = Synthetic: CDR-H2, CD39
GIIPAFGTANYAQKFQG

SEQ ID NO: 325 - protein, 17 aa, synthetic construct
  REGION 1..17: note = Synthetic: CDR-H2, CD39
MINPSGGSTKYAQKFQG

SEQ ID NO: 326 - skipped

SEQ ID NO: 327 - protein, 17 aa, synthetic construct
  REGION 1..17: note = Synthetic: CDR-H3, CD39
GPRYDSSGYRWRYGMDV

SEQ ID NO: 328 - protein, 12 aa, synthetic construct
  REGION 1..12: note = Synthetic: CDR-H3, CD39
EAGYYRYRYFDL

SEQ ID NO: 329 - protein, 12 aa, synthetic construct
  REGION 1..12: note = Synthetic: CDR-H3, CD39
EAGYYRYRYFDL

SEQ ID NO: 330 - protein, 10 aa, synthetic construct
  REGION 1..10: note = Synthetic: CDR-H3, CD39
DPVRRSPFDI

SEQ ID NO: 331 - protein, 15 aa, synthetic construct
  REGION 1..15: note = Synthetic: CDR-H3, CD39
DAPFYTWDHYYGMDV

SEQ ID NO: 332 - skipped

SEQ ID NO: 333 - protein, 11 aa, synthetic construct
  REGION 1..11: note = Synthetic: CDR-L1, CD39
RASQSISSYLN

SEQ ID NO: 334 - protein, 11 aa, synthetic construct
  REGION 1..11: note = Synthetic: CDR-L1, CD39
RASQSVSSNLA

SEQ ID NO: 335 - protein, 11 aa, synthetic construct
  REGION 1..11: note = Synthetic: CDR-L1, CD39
RASQSVSSNLA

SEQ ID NO: 336 - protein, 11 aa, synthetic construct
  REGION 1..11: note = Synthetic: CDR-L1, CD39
RASQSVSSYLA

SEQ ID NO: 337 - protein, 11 aa, synthetic construct
  REGION 1..11: note = Synthetic: CDR-L1, CD39
QASQDISNYLN

SEQ ID NO: 338 - skipped

SEQ ID NO: 339 - protein, 7 aa, synthetic construct
  REGION 1..7: note = Synthetic: CDR-L2, CD39
AASSLQS

SEQ ID NO: 340 - protein, 7 aa, synthetic construct
  REGION 1..7: note = Synthetic: CDR-L2, CD39
GASTRAT

SEQ ID NO: 341 - protein, 7 aa, synthetic construct
  REGION 1..7: note = Synthetic: CDR-L2, CD39
GASTRAT

SEQ ID NO: 342 - protein, 7 aa, synthetic construct
  REGION 1..7: note = Synthetic: CDR-L2, CD39
DSSNRAT

SEQ ID NO: 343 - protein, 7 aa, synthetic construct
  REGION 1..7: note = Synthetic: CDR-L2, CD39
DASNLAT

SEQ ID NO: 344 - skipped

SEQ ID NO: 345 - protein, 10 aa, synthetic construct
  REGION 1..10: note = Synthetic: CDR-L3, CD39
QQLYVDPPWT

SEQ ID NO: 346 - protein, 9 aa, synthetic construct
  REGION 1..9: note = Synthetic: CDR-L3, CD39
QQHALWPLT

SEQ ID NO: 347 - protein, 9 aa, synthetic construct
  REGION 1..9: note = Synthetic: CDR-L3, CD39
QQHALWPLT

SEQ ID NO: 348 - protein, 9 aa, synthetic construct
  REGION 1..9: note = Synthetic: CDR-L3, CD39
QQSFLWPRT

SEQ ID NO: 349 - protein, 9 aa, synthetic construct
  REGION 1..9: note = Synthetic: CDR-L3, CD39
QQLYHLPIT

SEQ ID NO: 350 - skipped

SEQ ID NO: 351 - protein, 124 aa, synthetic construct
  REGION 1..124: note = Synthetic: VH, CD39 (SRF360)
EVQLVESGGGLVKPGGSLRLSCAASGFTFSSYRMNWVRQAPGKGLEWVSSISSSSSSIWYADSVKGRFTISRDNAKNSLYLQMNSLRAEDTAVYYCAKGPRYDSSGYRWRYGMDVWGQGTTVSS

SEQ ID NO: 352 - protein, 108 aa, synthetic construct
  REGION 1..108: note = Synthetic: VL, CD39 (SRF360)
DIQMTQSPSSLSASVGDRVTITCRASQSISSYLNWYQQKPGKAPKLLIYAASSLQSGVPSRFSGSGSGTDFTLTISSLQPEDFATYYCQQLYVDPPWTFGGGTKVEIK

SEQ ID NO: 353 - protein, 451 aa, synthetic construct
  REGION 1..451: note = Synthetic: HC, CD39 (SRF360)-G4
EVQLVESGGGLVKPGGSLRLSCAASGFTFSSYRMNWVRQAPGKGLEWVSSISSSSSSIWYADSVKGRFTISRDNAKNSLYLQMNSLRAEDTAVYYCAKGPRYDSSGYRWRYGMDVWGQGTTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 354 - protein, 215 aa, synthetic construct
  REGION 1..215: note = Synthetic: LC, CD39 (SRF360)
DIQMTQSPSSLSASVGDRVTITCRASQSISSYLNWYQQKPGKAPKLLIYAASSLQSGVPSRFSGSGSGTDFTLTISSLQPEDFATYYCQQLYVDPPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 355 - protein, 121 aa, synthetic construct
  REGION 1..121: note = Synthetic: VH, CD39 (SRF365)
QVQLVQSGAEVKKPGSSVKVSCKASGGTFSDKAISWVRQAPGQGLEWMGSILPIFGTANYAQKFQGRVTITADESTSTAYMELSSLRSEDTAVYYCAREAGYYRYRYFDLWGRGTLVTVSS

SEQ ID NO: 356 - protein, 214 aa, synthetic construct
  REGION 1..214: note = Synthetic: VL, CD39 (SRF365)
EIVMTQSPATLSVSPGERATLSCRASQSVSSNLAWYQQKPGQAPRLLIYGASTRATGIPARFSGSGSGTEFTLTISSLQSEDFAVYYCQQHALWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - protein, 448 aa, synthetic construct
  REGION 1..448: note = Synthetic: HC, CD39 (SRF365)
QVQLVQSGAEVKKPGSSVKVSCKASGGTFSDKAISWVRQAPGQGLEWMGSILPIFGTANYAQKFQGRVTITADESTSTAYMELSSLRSEDTAVYYCAREAGYYRYRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 358 - protein, 214 aa, synthetic construct; identical to SEQ ID NO: 356
  REGION 1..214: note = Synthetic: LC, CD39 (SRF365)

SEQ ID NO: 359 - protein, 121 aa, synthetic construct
  REGION 1..121: note = Synthetic: VH, CD39 (SRF367)
QVQLVQSGAEVKKPGSSVKVSCKASGGTFSSEGISWVRQAPGQGLEWMGSILPIFGTANYAQKFQGRVTITADESTSTAYMELSSLRSEDTAVYYCAREAGYYRYRYFDLWGKGTLVTVSS

SEQ ID NO: 360 - protein, 107 aa, synthetic construct
  REGION 1..107: note = Synthetic: VL, CD39 (SRF367)
EIVMTQSPATLSVSPGERATLSCRASQSVSSNLAWYQQKPGQAPRLLIYGASTRATGIPARFSGSGSGTEFTLTISSLQSEDFAVYYCQQHALWPLTFGGGTKVEIK

SEQ ID NO: 361 - protein, 448 aa, synthetic construct
  REGION 1..448: note = Synthetic: HC, CD39 (SRF367)
QVQLVQSGAEVKKPGSSVKVSCKASGGTFSSEGISWVRQAPGQGLEWMGSILPIFGTANYAQKFQGRVTITADESTSTAYMELSSLRSEDTAVYYCAREAGYYRYRYFDLWGKGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 362 - protein, 214 aa, synthetic construct; identical to SEQ ID NO: 356
  REGION 1..214: note = Synthetic: LC, CD39 (SRF367)

SEQ ID NO: 363 - protein, 119 aa, synthetic construct
  REGION 1..119: note = Synthetic: VH, CD39 (SRF370)
QVQLVQSGAEVKKPGSSVKVSCKASGGTFSTYAIGWVRQAPGQGLEWMGGIIPAFGTANYAQKFQGRVTITADESTSTAYMELSSLRSEDTAVYYCARDPVRRSPFDIWGQGTMVTVSS

SEQ ID NO: 364 - protein, 107 aa, synthetic construct
  REGION 1..107: note = Synthetic: VL, CD39 (SRF370)
EIVLTQSPATLSLSPGERATLSCRASQSVSSYLAWYQQKPGQAPRLLIYDSSNRATGIPARFSGSGSGTDFTLTISSLEPEDFAVYYCQQSFLWPRTFGGGTKVEIK

SEQ ID NO: 365 - protein, 446 aa, synthetic construct
  REGION 1..446: note = Synthetic: HC, CD39 (SRF370)
QVQLVQSGAEVKKPGSSVKVSCKASGGTFSTYAIGWVRQAPGQGLEWMGGIIPAFGTANYAQKFQGRVTITADESTSTAYMELSSLRSEDTAVYYCARDPVRRSPFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 366 - protein, 214 aa, synthetic construct
  REGION 1..214: note = Synthetic: LC, CD39 (SRF370)
EIVLTQSPATLSLSPGERATLSCRASQSVSSYLAWYQQKPGQAPRLLIYDSSNRATGIPARFSGSGSGTDFTLTISSLEPEDFAVYYCQQSFLWPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 367 - protein, 124 aa, synthetic construct
  REGION 1..124: note = Synthetic: VH, CD39 (SRF399)
QVQLVQSGAEVKKPGASVKVSCKASGYTFSSWYMHWVRQAPGQGLEWMGMINPSGGSTKYAQKFQGRVTMTRDTSTSTVYMELSSLRSEDTAVYYCARDAPFYTWDHYYGMDVWGQGTTVTVSS

SEQ ID NO: 368 - protein, 107 aa, synthetic construct
  REGION 1..107: note = Synthetic: VL, CD39 (SRF399)
DIQMTQSPSSLSASVGDRVTITCQASQDISNYLNWYQQKPGKAPKLLIYDASNLATGVPSRFSGSGSGTDFTFTISSLQPEDIATYYCQQLYHLPITFGGGTKVEIK

SEQ ID NO: 369 - protein, 451 aa, synthetic construct
  REGION 1..451: note = Synthetic: HC, CD39 (SRF399)
QVQLVQSGAEVKKPGASVKVSCKASGYTFSSWYMHWVRQAPGQGLEWMGMINPSGGSTKYAQKFQGRVTMTRDTSTSTVYMELSSLRSEDTAVYYCARDAPFYTWDHYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 370 - protein, 214 aa, synthetic construct
  REGION 1..214: note = Synthetic: LC, CD39 (SRF399)
DIQMTQSPSSLSASVGDRVTITCQASQDISNYLNWYQQKPGKAPKLLIYDASNLATGVPSRFSGSGSGTDFTFTISSLQPEDIATYYCQQLYHLPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC